(19) United States
(12) Patent Application Publication
(10) Pub. No.: US 2025/0290066 A1
(43) Pub. Date: Sep. 18, 2025

(54) COMPOSITIONS FOR THE SILENCING OF snoRNAS AND METHODS OF USING SAME

(71) Applicant: Duke University, Durham, NC (US)

(72) Inventors: Christopher HOLLEY, Durham, NC (US); Brittany ELLIOTT, Durham, NC (US); Neil FREEDMAN, Durham, NC (US)

(21) Appl. No.: 18/861,505

(22) PCT Filed: May 1, 2023
(86) PCT No.: PCT/US2023/066460

§ 371 Date: Oct. 29, 2024

Related U.S. Application Data
(60) Provisional application No. 63/336,578, filed on Apr. 29, 2022.
(60) Provisional application No. 63/397,076, filed on Aug. 11, 2022.

(51) Int. Cl. C12N 15/113; A61P 9/10; A61P 29/00

(52) U.S. Cl. CPC C12N 15/113; A61P 9/10; A61P 29/00; C12N 2310/113; C12N 2310/315; C12N 2310/321; C12N 2310/3341

(57) ABSTRACT

The present disclosure describes, in part, compositions and methods for preventing and treating cardiovascular disease, atherosclerosis and inflammation by inhibiting or decreasing production, expression or activity of Rpl13 snoRNA.

### CROSS-REFERENCE TO RELATED APPLICATIONS

This application claims priority to U.S. Provisional Application Nos. 63/336,578 filed on Apr. 29, 2022, and 63/397,076 filed on Aug. 11, 2022, the contents of which are incorporated by reference in their entireties.

### STATEMENT REGARDING FEDERALLY SPONSORED RESEARCH

This invention was made with government support under grant number UL1TR002553 awarded by the National Institutes of Health. The government has certain rights in this invention.

### REFERENCE TO AN ELECTRONIC SEQUENCE LISTING

The contents of the electronic sequence listing (155554.00694.xml; Size: 192,555 bytes; and Date of Creation: May 1, 2023) is herein incorporated by reference in its entirety.

### BACKGROUND

Atherosclerosis is a leading cause of morbidity and mortality worldwide, affecting more than 500 million individuals globally and accounting for 19 million deaths annually. In the United States, Atherosclerosis afflicts about 26 million people, and results in 2 million hospitalizations and 400,000 deaths every year. There is no question that prevention and treatment of atherosclerosis is a public health priority of the highest order.

Most currently available drugs to treat or prevent atherosclerosis lower levels of LDL cholesterol (LDL-C). For example, an estimated 200 million patients currently take statins, which lower LDL-C and reduce the risk of atherosclerosis events by approximately 30%. For those patients in whom statin therapy is unable to reduce LDL-C to ever-tighter goal levels (now <55 mg/dL), the development of PCSK9 inhibitors represent a promising second-line therapy (monoclonal antibodies alirocumab and evolocumab, and the siRNA therapeutic inclisiran). However, clinical use of PCSK9 inhibitors has been limited due to high cost and the fact that they do what statins do (reduce LDL-C), making them second-line therapies for use only if statins are insufficient or poorly tolerated. Other lipid-targeted drugs have also been developed to combat atherosclerosis some with no success (fibrates), some with modest success (ezetimibe, icosapent ethyl, bempedoic acid). While LDL is a critical component, atherosclerosis is more broadly an inflammatory disease that involves the accumulation of lipids and inflammatory cells in medium to large arteries. Dyslipidemia from high-fat diet (aka Western diet) is a source of arterial cholesterol, but related mechanisms play critical roles in atherosclerosis, and they are not directly addressed by current mainstream therapies. Accordingly, there is a remaining need in the art for additional atherosclerosis therapies in particular those that target cholesterol associated inflammation.

### SUMMARY

Described herein, the inventors have developed a novel strategy to address atherosclerosis, in particular the present invention provides compositions, methods and kits for the treatment of atherosclerosis with antisense oligonucleotides. These antisense oligonucleotides were also found to have broader anti-inflammatory effects and may be useful in treating a wider array of inflammatory diseases or conditions associated with elevated inflammation.

In one aspect the present invention provides a composition or pharmaceutical composition, comprising an antisense oligonucleotide capable of binding an Rpl13a snoRNA. The antisense oligonucleotide comprises at least one of SEQ ID NO: 17-32 or 77-84, a sequence with at least 90% or 95% identity to SEQ ID NO: 17-32 or 77-84, a sequence of SEQ ID NO: 17-32 or 77-84 modified to increase its stability, and combinations thereof. In some embodiments the antisense oligonucleotide is DNA or modified DNA to increase stability of the antisense oligonucleotide. In some embodiments, the antisense oligonucleotide is modified to comprise a phosphorothioate backbone, 5-methylcytosines and the first five 5′ and last five 3′ nucleotides comprise 2′-O-methoxy-ethyl bases (2′-MOE) bases. In some embodiments, the oligonucleotide comprises at least one of SEQ ID NO: 33-72 or sequences having at least 90% or 95% identity to at least one of SEQ ID NO: 33-72. In some embodiments, the composition additionally comprises at least one of SEQ ID NO: 1-4 or 73-76 and combinations thereof. In one embodiment, the Rpl13a snoRNA comprises at least one of U32a, U33, U34 or U35a.

A second aspect of the present disclosure provides a method of treating and/or preventing cardiovascular disease in a subject in need thereof. The method comprises administering a therapeutically effective amount of an inhibitor of a Rpl13a snoRNA. In some embodiments, the method decreases and or reduces inflammation in the subject as compared to the subject prior to treatment or as compared to a similar control subject, who was not administered the inhibitor. In some embodiments, the Rpl13a snoRNA comprises at least one of or all four of U32a, U33, U34, or U35a. In some embodiments, the inhibitor is selected from SEQ ID NO: 17-84, SEQ ID NO: 1-4, sequences having at least 90% or 95% sequence identity to SEQ ID NOs: 1-4 or 17-84 or combinations thereof.

A third aspect of the present disclosure provides a method of preventing and/or treating inflammation in a subject. The method comprises administering a therapeutically effective amount of an inhibitor of a Rpl13a snoRNA. In some embodiments the snoRNA comprises U32a, U33, U34 or U35 or combinations thereof. In some embodiments the inhibitor is selected from SEQ ID NO: 17-84, SEQ ID NO: 1-4, sequences having at least 90% or 95% sequence identity to at least one of SEQ ID NO: 1-4 or 17-84 or combinations thereof. In some embodiments, the inflammation is associated with atherosclerosis and/or cardiovascular disease. In some embodiments, the administration results in decreased IL-1B as compared to the subject prior to administration of the inhibitor or as compared to a control subject who as not administered the inhibitor.

Another aspect of the present invention provides a vector comprising a promoter operably connected to a nucleic acid sequence encoding at least one of SEQ ID NO: 17-32 or 77-84.

Another aspect of the present invention provides a kit comprising at least four antisense oligonucleotides selected from the group consisting of SEQ ID NOs: 1-4 and 17-84. In some embodiments the kit comprises at least one antisense oligonucleotide capable of binding to each of U32a, U33, U34 or U35a.

### BRIEF DESCRIPTION OF THE DRAWINGS

The present technology can be better understood by reference to the following drawings. The drawings are merely exemplary to illustrate certain features that may be used singularly or in combination with other features and the present technology should not be limited to the embodiments shown.

FIG. 1. Schematic showing strategies for inhibiting snoRNAs via targeted antisense oligonucleotides (ASOs) (A) snoRNA are highly structured, short non-coding RNAs with conserved Box C and Box D regions in accordance with one embodiment of the present disclosure. ASOs (in red) designed to target the antisense element (ASE) of target snoRNA can sterically inhibit function (B), or recruit degradation of snoRNA by RNase H that cleaves the RNA strand of RNA-DNA duplexes (C).

FIG. 2. Schematic showing human RPL13a snoRNA-targeted ASO design. Human RPL13a snoRNA-targeted ASO design. ASOs complementary to RNA target are 20 nucleotides in length. RNA or DNA nucleotides (A) are chemically modified to provide protection from nucleases and increase binding affinity. The phosphothioate (PS) modification (B) is used across all nucleotides and protects the molecule from nucleases. 2′-O-methoxyethyl (MOE) is used to increase binding affinity to the RNA target and protect against nucleases. ASO “gapmer” (C) design includes five MOE bases at both the 5′ and 3′ ends, which is optimized for recruiting RNase H cleavage of RNA target in the center 10 nucleotides.

FIG. 3. Rpl13a snoRNAs promote athero and SMC-to-foam-cell transdifferentiation. Brachiocephalic arteries (BCAs) were harvested from congenic 8-wk-old female Apoe−/− and snoKO/Apoe−/− mice fed a Western diet for 14 wk.1 Serial frozen sections were stained concurrently with (a) Cy3-conjugated IgG targeting either SMC α-actin (ACTA2) or no protein (neg control), (b) BODIPY® 493/503 (neutral lipids, green), and (c) Hoechst 33342 (DNA, blue). 1 Confocal images were acquired at an optical slice thickness of 1 μm; all samples used identical camera settings, as we reported. 1 Isotype control IgG yielded no color (not shown). A, Photomicrographs depict BCA cross sections (left); dashed boxes denote areas enlarged in rightward panels; dotted line designates internal elastic lamina (IEL). Co-localization of BODIPY with DNA (white) or BODIPY with ACTA2 (yellow) was performed with Imaris 9.2 software. 1 Foam cells (as opposed to extracellular lipid) were identified as DNA-associated BODIPY.1 “L,” Lumen. Scale bars=50 μm. B, Neointimal, medial and luminal areas were plotted (with means±SE) from 8 distinct mice of each genotype. Compared with Apoe−/−: *, p<0.01 (2-way ANOVA with Sidak test for multiple comparisons). C, Neointimal foam cells (≥100 counted/artery) were divided by the total # of neointimal cells to obtain foam cell prevalence for distinct BCAs (with means±SE). Compared with Apoe−/−: *, p<0.02. “Yellow” neointimal foam cells (i.e., containing ACTA2) were plotted as a % of total neointimal foam cells for 6 distinct BCAs per genotype (with means±SE). Compared with Apoe−/−: *, p<0.001. D, The area of carotid neointimal cross sections occupied by the indicated component was measured by planimetry (Image J) and normalized to total neointimal area to obtain “% of neointima” (means±SE). Necrotic core was identified as we reported. Compared with Apoe−/−: *, p<0.02 (Mann-Whitney tests, p values corrected for multiple comparisons [GraphPad Prism 9.2]). All quantitation was blinded with regard to specimen identity.

FIG. 4. Rpl13a snoRNAs exacerbate athero. Common carotid arteries from WT or snoKO (snoRNA KO) mice were orthotopically transplanted into congenic Apoe−/− mice and harvested 6 wk post-op after perfusion fixation. Cross sections from the middle of each artery were stained with a modified trichrome stain; serial sections were stained for DNA (blue) and smooth muscle α-actin (SMA, red). Scale bars=100 μm. Neointimal, medial and cross-sectional areas were measured by an observer blinded to specimen identity with Image J, and plotted as mean±SE from ≥8 carotid arteries of each genotype. Compared with WT arteries: *, p<0.01.

FIG. 5. Rpl13a snoRNAs augment SMC ROS levels, proliferation, migration, and inflammation. Primary aortic SMCs were isolated from congenic C57BL/6J WT and snoKO mice. All data are from ≥3 independently isolated SMC lines of each genotype. A, Confluent SMCs in growth medium were loaded with 2′,7′-dichlorodihydrofluorescein diacetate (DCF-2, 1 μM) for 30 min or with MitoSOX™ Red (2.5 μM) for 10 min (37° C.), trypsinized and subjected to flow cytometry. DCF or MitoSOX fluorescence (ROS read-out) is plotted as median values for fluorescence per SMC for 5-6 experiments. Compared with WT: *, p<0.03. B, SMCs grown in medium containing 2.5% FBS were counted at the indicated time points. Shown are means±SE from 3 experiments. Compared with WT: *, p<0.01. C, SMCs were subjected to migration assays in modified Boyden chambers with serum-free medium lacking (None) or containing 1 nM PDGF-BB, as we described. The absorbance of crystal violet eluted from migrated SMCs was multiplied ×10 and plotted for 5 independent experiments. Compared with WT: *, p<0.01. (All p values from 2-way ANOVA with Sidak post-hoc test for multiple comparisons [data passed normality tests].) D, SMCs were incubated for 24 hr (37° C.) in serum-free medium lacking (“None”) or containing lipopolysaccharide (LPS, 100 ng/ml) or murine TNF (10 ng/ml), and then solubilized. Serial immunoblotting for VCAM-1 and β-actin was performed as reported. Results are representative of 3 independent experiments with LPS and TNF. VCAM-1 band densities were normalized to cognate actin band densities; ratios were analyzed by 2-way ANOVA with Tukey post-hoc test): VCAM-1 bands were 2.0±0.5-fold greater in WT than in snoKO SMCs (p<0.03).

FIG. 6. snoRNAs augment endothelial cell inflammation and Mφ ROS levels. A, Primary aortic endothelial cells (ECs) from 3 WT and 3 snoKO mice were isolated and cultured as reported. 8 Confluent ECs in 1% FBS medium were then cultured for 16 h with the following “flow” conditions: static (“−”), or disturbed (“+”, orbital shaker at 100 rpm, 2-4 dyn/cm2). Next, ECs were solubilized and immunoblotted serially for VCAM-1 and actin. VCAM-1 band densities were normalized to cognate β-actin bands; ratios for 2 independent experiments showed 3=1-fold greater VCAM-1 expression in WT than in snoKO ECs stimulated with flow (p<0.05; 2-way ANOVA, Sidak posthoc test). B, Congenic snoKO and WT bone marrow-derived Mφs11 were plated at 20×104/cm² in growth medium and stimulated with LPS (100 ng/ml) and interferon-γ (10 ng/ml) for 16 hr for M1 polarization. Mφs were then loaded with MitoSOX (2.5 μM) 17 for 10 min (37° C.). Mφs were fixed (2 min), stained for DNA and then imaged. Scale bars=50 μm. All cells were CD68+ (not shown). C, Red MitoSOX fluorescence was normalized to cognate DNA fluorescence, to obtain “fluorescence/cell” in 2 microscopic fields per Mφ line (≥200 Mφs/field), with ImageJ by an observer blinded to specimen identity. MitoSOX fluorescence per cell is plotted, along with means±SE, from 3 distinct Mφ lines of each genotype. Compared with WT Mφs: *, p<0.01.

FIG. 7. COX4i2 silencing in WT and snoKO SMCs. WT and snoKO SMCs were transfected (as reported) with siRNA targeting no known mouse mRNA (control, “Ctl”) or Cox412, and then serially immunoblotted 72 hr later for COX4i2 and β-actin. Results from a single experiment, representative of 3 experiments performed with independently isolated WT and snoKO SMCs (all distinct from those used in the proteomics experiments in the Table 1).

FIG. 8. Human snoRNA U32A facilitates 2′-O-methylation of COX4i2. CRISPR/Cas9 was used on (human) HEK293T cells to knock out (“KO”) snoRNAs U25 or U32A and U51. mRNA harvested from 3 clones of each cell line (or the parental cell line, “WT”) was subjected to reverse transcription (RT) at low [dNTP] followed by quantitative PCR (RTL-P) of COX4i2, to demonstrate the presence of Nm sites as reported. Higher RTL-P efficiency on COX4i2 mRNA obtained from U32A/U51 KO cells indicates reduced Nm modification on COX4i2 mRNA in these cells. Plotted are results from 3 independent clones of each line, with means±SE. Compared with WT.

FIG. 9. Loss of Rpl13a snoRNAs leads to lower mitochondrial oxygen consumption. Aortic smooth muscle cells (SMC) were isolated from WT and snoKO mice, cultured in low glucose (5.5 mM), and subjected to mitochondrial stress testing on the Seahorse platform. Oxygen consumption rate (OCR) is normalized to mitochondrial content (MitoTracker fluorescence). Relative OCR is shown for both basal and maximal (uncoupled) mitochondrial metabolism. Both basal and maximal OCR are reduced by ≥50% in snoKO SMCs. ** p<0.05, n=7-8/group.

FIG. 10. Rpl13a snoRNAs promote athero and SMC-to-foam-cell transdifferentiation. Congenic 10-wk-old male Apoe−/− and Rpl13a-snoRNA−/+/Apoe−/− mice had wire-mediated carotid artery de-endothelialization and then ate Western diet for 6 wk, as we reported. 13 Serial frozen sections were stained concurrently with (a) Cy3-conjugated IgG targeting either smooth muscle (SM) α-actin or no protein (neg control), (b) BODIPY® 493/503 (neutral lipids, green), and (c) Hoechst 33342 (DNA, blue). Images were acquired with a Leica SP8 confocal microscope, at an optical slice thickness of 1 μm; all samples used identical camera settings. Isotype control IgG yielded no color (not shown). A, Photomicrographs depict the internal elastic lamina (IEL) as a dotted line. The boxed area in panel 4 is enlarged in panel 5. Co-localization of BODIPY with DNA (white) or BODIPY with SM α-actin (yellow) was performed with Imaris 9.2 software. Foam cells (as opposed to extracellular lipid) were identified as DNA-associated BODIPY.1 “L,” Lumen. Leftmost: total carotid cross section. Scale bars=50 μm. B, Neointimal, medial and luminal areas were plotted (with means±SE) from 5 distinct mice of each genotype. Compared with Apoe−/−: *, p<0.03 (Mann-Whitney, Holm-Sidak correction for multiple comparisons). All quantitation was blinded with regard to specimen identity.

FIG. 11. Rpl13a snoRNAs in arterial wall cells promote athero and SMC-to-foam-cell transdifferentiation. Common carotid arteries from WT or snoKO (snoRNA KO) mice were orthotopically transplanted into congenic Apoe−/− mice, harvested 6 wk post-op and frozen in OCT compound. Serial sections were stained with anti-apoE goat IgG and then simultaneously with anti-goat/Alexa-546 (red), BODIPY® 493/503 (for neutral lipids, green), and Hoechst 33342 (for DNA, blue). Serial sections stained with nonimmune goat IgG showed no red color. Image acquisition employed a Leica SP8 confocal microscope with an optical slice thickness of 1 μm; identical camera settings were used for each sample. A, Photomicrographs were obtained at the indicated magnification; the internal elastic lamina (IEL) is demarcated by a dotted line. The boxed area in panel 4 is enlarged in panel 5. Co-localization of red (apoE) with either blue (Hoechst) or green (BODIPY) was performed using Imaris 9.2 software, to yield white (not shown) or yellow, respectively. “L,” Lumen. The total carotid graft cross section appears at in the leftmost panel. Scale bars=30 μm. B, BODIPY-stained material was judged to be cellular if it co-localized with nuclei (designated white, not shown); neointimal BODIPY+ (foam) cells (≥100 counted per carotid graft neointima) were divided by the total number of neointimal cells to obtain foam cell prevalence, plotted for distinct carotids along with means±SE. Compared with WT: *, p<0.01. BODIPY+neointimal cells (≥100 per carotid graft) were scored as “yellow” (i.e., containing some yellow elements) or green by an observer blinded to specimen identity; the percentage of BODIPY+ (foam) cells that are yellow was plotted for 6 distinct carotids, along with means±SE. Compared with WT: *, p<0.01.

FIG. 12. Rpl13a snoRNAs up-regulate steady-state arterial ROS levels and aggravate neointimal hyperplasia induced by endothelial injury. A, Frozen sections of aorta and carotid arteries (not shown) from WT and snoKO mice were cut at 10 μm and incubated with 5 mM L-NAME, ±CellROX® Orange (5 μm), as we described. Fluorescent images were captured at identical microscope and CCD camera settings for all samples. Shown are fluorescence photomicrographs from single aortas of each genotype. Serial sections incubated without CellROX® showed no fluorescence (not shown). Scale bars=100 μm. With Image J, CellROX® fluorescence (red pixels/mm²) from the entire aorta was measured. Values from three distinct aortas of each genotype were plotted, along with means±SE. Compared with WT: *, p<0.01. Carotid arteries provided concordant data (not shown). B, Congenic mice of the indicated genotype were euthanized 4 weeks after wire-mediated carotid artery de-endothelialization.²⁻⁵ Perfusion-fixed carotids were stained with a modified connective tissue stain (“Trichrome”) or immunofluorescently for smooth muscle α-actin (ACTA2, with DNA counterstain). Samples shown represent 5 mice of each genotype. Scale bars=200 μm. C, Neointimal, medial and luminal areas³ were plotted, along with means±SE from 5 distinct mice of each genotype. Compared with WT arteries: *, p<0.01 (w-way ANOVA with Tukey post-hoc test for multiple comparisons). Measurements were made by observers blinded to specimen identity.

FIG. 13. Rpl13a snoRNAs augment arterial inflammation. Carotid arteries subjected to endothelial denudation (FIG. 9) were serially sectioned, and individual sections were immunostained for the NFκB p65 subunit phosphorylated on Ser536 (“phospho-p65,” an activating phosphorylation site), the NFκB gene product vascular cell adhesion molecule-1 (VCAM-1), total p65, or no specific protein (isotype control IgG), and counterstained with Hoechst 33342 (DNA, blue). All sections were imaged with identical confocal microscope and camera settings; dotted lines indicate the external elastic lamina of each artery. Scale bars=20 μm. Protein-specific immunofluorescence was normalized to cognate DNA fluorescence in each microscopic field (and microscopic fields covered the entire carotid artery cross section). The ratio of protein/DNA was plotted (arbitrary units) for carotids from 5 mice of each genotype, and plotted (along with means±SE). Compared with WT: *, p<0.01 (w-way ANOVA with Tukey post-hoc test for multiple comparisons). Measurements were made by observers blinded to specimen identity.

FIG. 14. Rpl13a snoRNAs augment SMC proliferation in vivo. Serial sections of carotid arteries used in FIGS. 9 and 10 were immunostained for proliferating cell nuclear antigen (PCNA, green) and smooth muscle α-actin (ACTA2, red), and counterstained for DNA (blue). Isotype control IgGs yielded no green or red color (not shown). Co-localization of PCNA with ACTA2 was performed using Imaris 9.2 software, to yield yellow. These PCNA⁺ yellow cells were counted in each microscopic field and normalized to the total number of ACTA2⁺ cells (SMCs) in each field; ≥100 PCNA⁺ cells per carotid were counted. The ratio of PCNA⁺ to total SMCs was plotted for carotid arteries from 5 mice of each genotype. Compared with WT: *, p<0.02 (t test).

FIG. 15. Rpl13a-snoRNAs augment inflammatory signaling in vascular smooth muscle cells (SMCs). Primary SMCs of the indicated genotype were stimulated (or not) with murine tumor necrosis factor (TNF) at 10 ng/ml for 10 min (37° C.), and solubilized. SMC protein extracts were resolved by SDS-PAGE and immunoblotted serially for the Ser-536-phosphorylated isoform of the NFκB subunit p65 (p-p65) and β-actin. (Phosphorylation of p65 on Ser536 enhances its transcriptional activity, and can be used as a readout for the activity of IκB kinase-β.) Bands for p-p65 were normalized to cognate β-actin bands, and these ratios were plotted (arbitrary units) for 3 distinct lines of primary SMCs of each genotype (along with means±SE). Compared with WT: *, p<0.05.

FIG. 16. Rpl13a snoRNAs downregulate COX4i2 in mouse SMCs and aortas as well as in human cells. A, SMCs of the indicated genotype were grown to confluence and then solubilized. SMC proteins were resolved by SDS-PAGE and immunoblotted serially for COX4i2 and β-actin; parallel immunoblots were probed with isotype control IgG (“Control”). Band intensities for COX4i2 were normalized to cognate β-actin bands and plotted (arbitrary units) for 4 independently isolated primary SMC lines of each genotype (along with means±SE). Compared with WT: *, p<0.02 (t test). B, Aortas from mice of the indicated genotype were solubilized immediately after harvest from euthanized mice. Protein extracts from each aorta were subjected to SDS-PAGE and then immunoblotted for COX4i2. Band intensities for COX4i2 were normalize to cognate total protein band intensities and plotted (along with means±SE) for aortas from 5 mice of each genotype. Compared with WT: *, p<0.01 (t test). C, Human embryonic kidney 293T (HEK293T) cells were processed by CRISPR/Cas9 to delete either U25 (an irrelevant snoRNA) or the Rpl13a-snoRNA U32a and its congener U51. Cells were grown in full growth medium to confluence, and then solubilized. Protein extracts were resolved by SDS-PAGE and immunoblotted serially for COX4i2 and β-actin; parallel immunoblots were probed with isotype control IgG (“Control”). Band intensities for COX4i2 were normalized to cognate β-actin bands and plotted (along with means±SE, arbitrary units) for 3 independently isolated clones of each HEK293T knockout line. Compared with WT: *,p<0.02 (ttest). (D) While equivalent transcripts levels of COX4i2 are detected in WT, U25 KO (control), U32a KO, and U32a/U51 KO cells (top graph), decreased methylation of transcript is observed only in U32a/U51 KO cells. Results are presented as mean±S.E *p<0.05.

FIG. 17. In vivo ASO treatment reduces aortic Rpl13a snoRNA expression and athero without toxicity. (A) 10 wk C57BL/6 mice were injected with saline, ASO targeting Gfp (ctrl), or a pool of ASOs targeting the Rpl13a snoRNAs (sno). Total ASO dose was 48 mg/kg per mouse. Blood and aortas were collected after 1 wk, and aortic gene expression was measured by RT-qPCR (shown as relative expression, normalized to Rplp0 as a housekeeping gene). * p<0.05, ** p<0.01; n=3-4/group. (B) Blood samples from (A, “x1”), and additional samples from animals injected weekly x2 wks (“x2”) were measured for biomarkers of liver injury (AST, ALT) by ELISA. Normal and 95% (upper limit of normal, ULN) values for C57BL/6 mice are shown (Charles River Labs). All values were within the normal range and there were no statistically significant differences between saline, control ASO, and snoASO. (C) Male Apoe−/− mice were fed a Western diet starting at age 10 wk. Starting at age 24 wk (after established athero), mice were injected weekly with ASOs (48 mg/kg, SC) targeting either GFP (control) or all 4 Rpl13a-snoRNAs (“snoRNA”). Mice were sacrificed after 6 treatments, at age 30 wk. As in FIG. 3, brachiocephalic arteries were sectioned and stained for cholesterol (with BODIPY), smooth muscle α-actin (ACTA2) and DNA (DAPI); cross-sectional area of the atherosclerotic neointima and media were measured by observers blinded to specimen identity. The ratios of neointimal to medial areas are plotted (with means±SE) for 6-8 mice per ASO treatment group. Scale bar 100 μm. Compared with control: *, p<0.03. (D) Male and female Apoe−/− mice were fed a Western diet at 10 wk of age with concurrent treatment with ASOs (48 mg/kg, SC) targeting either GFP or snoRNAs. 4 injections were given weekly followed by 5 bi-weekly injections. At mouse harvest, mice were perfused with Oil Red O as previously described to visualize lesions. Lower aortas were harvested and anatomically pinned for en face imaging. Lesions were measured using ImageJ software and normalized to total area. Compared with control: *, p<0.02.

FIG. 18. IL-1ß levels are decreased in ASO treated mice. Decreased interleukin 1 beta (II 1-b) transcripts observed in the blood of Apoe−/− mice allowed to develop athero for 14 weeks prior to intervention with 48 mg/kg subcutaneous injection of ASOs targeting all 4 snoRNAs compared with GFP treated controls.

FIG. 19. After 14 weeks on HFD, Apoe−/− mice were treated with 48 mg/kg GFP (control) or an equal molar mix of each Rpl13a snoRNA ASOs with six consecutive weekly doses. Serum was collected from mouse terminal bleeds and tested for liver function enzymes AST and ALT. Values are compared to measurements published by Charles River screening of normal ranges for these enzymes. (A-B.) Most results were in the acceptable range even with an aggressive dosing schema.

FIG. 20. ASO design. ASOs designed to target snoRNAs in different areas to determine optimal RNA silencing. Regions were chosen based on RNA modeling to determine open regions (optimal) and structured regions or areas known to be occupied by RNA binding proteins (not optimal). Four ASOs with increasing dose ranges were assayed per each RNA target in Hela cells. Overall, targeting the 5′ and 3′ antisense element (ASE) performed best. EC50 ranged from 0.05-2.4 nM for knockdown efficacy across doses. U35a qPCR detection limitations affected the evaluation of U35a knockdown efficacy. SEQ ID NO: 5 is shown.

FIG. 21. U32a ASO function. Best U32a ASO function was evaluated by measuring (A.) efficacy of U32a knockdown, (B.) stable expression of host gene (Rpl13a) transcript, and (C.) level of cell toxicity with cell release of lactate dehydrogenase (LDH) as a reporter. Each experiment was performed in duplicate with two or more replicate experiments set up on different days. The best performing ASO sequence was determined using a weighted criterion: target knockdown=3, stable expression of host gene transcript=1, and level of cell toxicity=2. The following rankings resulted listed from best to worst: 5′ ASE, 3′ ASE, Center, 5′ End.

FIG. 22. U33 ASO function. Best U33 ASO function was evaluated by measuring (A.) efficacy of U33 knockdown, (B.) stable expression of host gene (Rpl13a) transcript, and (C.) level of cell toxicity with cell release of lactate dehydrogenase (LDH) as a reporter. Each experiment was performed in duplicate with two or more replicate experiments set up on different days. The best performing ASO sequence was determined using a weighted criterion: target knockdown=3, stable expression of host gene transcript=1, and level of cell toxicity=2. The following rankings resulted listed from best to worst: 3′ ASE, 5′ ASE, Center, 5′ End.

FIG. 23. U34 ASO function. Best U34 ASO function was evaluated by measuring (A.) efficacy of U34 knockdown, (B.) stable expression of host gene (Rpl13a) transcript, and (C.) level of cell toxicity with cell release of lactate dehydrogenase (LDH) as a reporter. Each experiment was performed in duplicate with two or more replicate experiments set up on different days. The best performing ASO sequence was determined using a weighted criterion: target knockdown=3, stable expression of host gene transcript=1, and level of cell toxicity=2. The following rankings resulted listed from best to worst: 3′ ASE, 5′ ASE, Center, 5′ End

### DETAILED DESCRIPTION

As described herein, the inventors have developed a novel strategy to address the ongoing problem of atherosclerosis in high-risk patients with documented atherosclerosis. In particular the inventors have discovered that certain small, noncoding RNAs called snoRNAs (small nucleolar RNAs) are critical mediators of metabolic stress, and that selective loss of these snoRNAs strongly mitigates atherosclerosis in mouse models, despite high levels of LDL-C. Because snoRNAs are non-coding RNAs, they are not easily targeted by small molecules, and the inventors have instead developed antisense oligonucleotides (ASO) that target them as therapeutics. The present invention provides compositions, methods and kits for the treatment of atherosclerosis with ASO. In addition, these ASOs are shown to reduce inflammation and thus the inventors have demonstrated that these ASOs may be used to treat other inflammatory diseases and as a more general treatment to reduce inflammation.

### Compositions:

In a first aspect, the present invention provides a composition capable of reducing and/or inhibiting a Rpl13a snoRNA in a cell or subject, the composition comprising at least one of SEQ ID NO: 17-32, 77-84 or combinations thereof or sequences with at least 90% or at least 95% identity to SEQ ID NO: 17-32, 77-84 and wherein the Rpl13a snoRNA is reduced and/or inhibited as compared to a control.

As used herein, “reducing” means an amount below, or less than the amount prior to treatment. As used herein, “inhibiting” means to control, prevent, restrain, arrest, or regulate the action, function or expression of snoRNAs. In some embodiments a composition described herein may reduce or inhibit the expression of Rpl13a snoRNA such that the expression of Rpl13a snoRNA is less following the administration of the composition as compared to the amount prior to administration of the compositions provided herein or as compared to control treated or non-treated subjects. The expression or activity of a snoRNA can be reduced or inhibited via binding to a complementary antisense oligonucleotide.

As used herein, a “control” is a comparison subject or sample. The control may be a sample or subject which is not exposed to a test composition or method or a sample or subject which is treated with an inactive or altered form of a composition, or a sample or subject prior to receiving treatment, exposure to a composition or method. As used herein a control may be a subject which has not been exposed to an ASO as described herein. A control may also be a state or level of a marker, for example a Rpl13a snoRNA in a subject prior to exposure to an ASO, not exposed to a ASO or exposed to a scrambled or inactive ASO as described herein.

Small nucleolar RNAs (snoRNAs) are a class of small RNA molecules that primarily guide chemical modifications of other RNAs, mainly ribosomal RNAs, transfer RNAs and small nuclear RNAs. There are two main classes of snoRNA, the C/D box snoRNAs, which are associated with methylation, and the H/ACA box snoRNAs, which are associated with pseudouridylation. SnoRNAs are also referred to as guide RNAs. Ribosomal protein L13a (Rpl13a) encodes a member of the L13P family of ribosomal proteins and is a component of the 60S ribosomal subunit. Mammalian loci for rpL13a contain four highly conserved intronic box C/D small nucleolar RNAs (snoRNAs) that are predicted to be processed during splicing of the rpL13a pre-mRNA transcript. These snoRNAs termed U32a, U33, U34, and U35a, are located within the introns of Rpl13a.

snoRNA U32a, U33, U34 and U35a are also known as SNORD32a, SNORD33, SNORD34 and SNORD35a respectively and are located in the nucleolus of a eukaryotic cell. These snoRNAs are a C/D box class of snoRNAs which contain the conserved sequence motifs known as the C box (UGAUGA) and the D box (CUGA). The box C/D snoRNAs are primarily known to guide post-transcriptional modifications, especially 2′-O-methylation, of ribosomal RNA and small nuclear RNA. U32a may comprise the sequence of SEQ ID NO: 5 in humans, U33 may comprise the sequence of SEQ ID NO: 6, U34 may comprise the sequence of SEQ ID NO: 7, U35a may comprise the sequence of SEQ ID NO: 8.

In some embodiments, Rpl13a snoRNA(s) are decreased. Rpl13a snoRNA may be decreased by any means known in the art. These include, but are not limited to, RNA-based RNA interference including siRNA, and shRNA, DNA-based RNA interference, including antisense oligonucleotides, non-homologous end joining, and CRISPR-mediated gene knockdown or knockout, including using dCas9 with or without addition proteins, Cas12a and Cas13 family enzymes, full or partial gene deletion or gene editing or mutation, non-homologous end joining or Transcription Activator-Like Effector Nucleases (TALENs).

In some embodiments, an antisense oligonucleotide (ASO) is used to reduce or inhibit the activity of the snoRNA. ASE and ASOs, are short, synthetic, chemically modified chains of nucleotides that have the potential to target any gene or nucleotide product of interest. Typically, an ASO is a single-stranded sequence complementary to the sequence of the target's messenger RNA (mRNA) within a cell. The ASO used herein, may be complementary to Rpl13a snoRNA, including U32a, U33, U34 or U35a (presented as SEQ ID NOs: 5-8). An ASO complementary to a single Rpl13a snoRNA may be used or ASO which target multiple Rpl13a snoRNA. The sequence of the ASO for U32a may comprise SEQ ID NO: 1, 17-20, 33-36, 49-50, 57-58, 65-66, 73 and 77-78; the sequence of the ASO for U33 may comprise SEQ ID NO: 2, 21-24, 37-40, 51-52, 59-60, 67-68, 74 and 79-80; the sequence of the ASO for U34 may comprise SEQ ID NO: 3, 25-28, 41-44, 53-54, 61-62, 69-70, 75 and 81-82; and the sequence of the ASO for U35a may comprise SEQ ID NO: 4, 29-32, 45-48, 55-56, 63-64, 71-72, 76 and 83-84.

The ASOs described herein comprises sequences complementary to Rpl13a snoRNA and may be RNA or DNA sequences. Binding of these sequences decrease, reduces or inhibits the expression of the complementary Rpl13a snoRNA. It will be appreciated by one of skill in the art that these ASO may be modified. By way of example, and not limitation, these modifications may increase stability of the ASO, modify the immune response to the ASO, alter the pharmacokinetics or therapeutic index of the ASO or decrease off-target effects of the ASO. Typical modifications include those to the phosphate backbone and ribose modifications. For example, modifications to the type of nucleotide linkage or backbone include phosphorothioate (PS) backbone. Other backbone modifications may include a stereodefined backbone configuration or mesylphosphoramidate (MsPA) linkages. ASO may comprise one of these backbone modifications, or a combination of two or more within the same oligo. Additional modifications may include nucleotide modifications. Nucleotide modifications may include, but are not limited to, 2′-O-methyl modified ribose (2′-OMe), 2′-O-methoxyethyl modified ribose (2′-MOE), 2′fluoro (2′-F), Locked nucleic acid (LNA), Constrained ethyl (cEt), Tricyclo-DNA (tcDNA), Phosphorodiamidate morpholino oligos (PMO), Peptide nucleic acid (PNA), 5-methyl-cytosine (m5° C.), and N-acetylgalactosamine (GalNAc) modifications. Additional modifications known in the art include 5′ and 3′ modifications. Typical 5′ modifications may include, without limitation, inverted deoxythymidine bases, addition of a linker sequence such as C6, addition of a cholesterol, addition of a reactive linker sequence which could be conjugated to another moiety such as a PEG. Typical 3′ modifications may include, without limitation, inverted deoxythymidine bases, and inverted abasic residues. Additional modifications may include those which allow for localization, for example, targeting the ASO to the liver or other organ or cellular space. These modifications may include, for example, a 5′-cholesterol-TEG modification. Any ASO described herein may comprise a cholesterol TEG or similar modification. In some embodiments, multiple modifications may be used in one ASO and individual nucleotides may be modified differently from other nucleotides in the ASO. For example, a PS backbone and 2′-OMe modified base where only the first and last 5 nucleotides are 2′-OMe modified. Modified oligos described herein include SEQ ID NO: 1-4 and 33-72.

Some of the ASO described herein contain 10 central nucleotides surrounded on the 5′ and 3′ end by 5 wing nucleotides (5+10+5 configuration, also known as a gapmer). In addition to the backbone and nucleotide modifications described herein, additional modifications may include changes in the nucleotide configuration. For example, a 5+8+5 configuration may be used, wherein the central nucleotide sequence is 8 nucleotides in length, as in SEQ ID NO: 49-56, 65-72 and 77-84. Oligo lengths may also differ based on particular modifications, for example PMO modified oligos may be 25-30 nucleotides in length.

In some embodiments the ASO may comprise a full phosphorothioate backbone, and first five 5′ and last five 3′ nucleotides are 2′-MOE and all cytosines are 5′-methyl modified as in SEQ ID NO: 33-56. In some embodiments, the ASO may comprise a cholesterol-TEG, a full phosphorothioate backbone, and first five 5′ and last five 3′ nucleotides are 2′-MOE modified and all cytosines are 5′-methyl modified.

In some embodiments the ASO may comprise a full phosphorothioate backbone, and first five 5′ and last five 3′ nucleotides are 2′MOE modified, the cytosines are 5-methyl modified and are 18 nucleotides in length as in SEQ ID NO: 49-56. In some embodiments the ASO may comprise a cholesterol-TEG, a full phosphorothioate backbone, first five 5′ and last five 3′ nucleotides are modified with 2′-MOE, cytosines are 5-methyl modified and are 18 nucleotides in length.

In some embodiments, the ASO may comprise a phosphodiester and phosphorothioate backbone, the first five 5′ and last five 3′ nucleotides are modified with 2′ MOE and cytosines are 5-methyl modified as in SEQ ID NO: 57-72. In some embodiments, the ASO may comprise a phosphodiester and phosphorothioate, backbone, a cholesterol-TEG, first five 5′ and last five 3′ nucleotides are modified with 2′-MOE and cytosines are 5-methyl modified.

In some embodiments, the ASO may comprise a phosphodiester and phosphorothioate, backbone, the first five 5′ and last five 3′ nucleotides are 2′MOE modified, the cytosines are 5-methyl modified and are 18 nucleotides in length as in SEQ ID NO: 65-72. In some embodiments, the ASO may comprise a phosphodiester and phosphorothioate backbone, a cholesterol-TEG, first five 5′ and last five 3′ nucleotides are modified with 2′-MOE, cytosines are 5-methyl modified and are 18 nucleotides in length.

The ASO described herein target a human Rpl13a snoRNA, including U32a, U33, U34, and U35. The ASO may target any portion of the Rpl13a snoRNA sequence, including binding at the 5′ end of the snoRNA sequence or 3′ end. In some embodiment, ASO which target more than one region within a single Rpl13a snoRNA may be used. For example, SEQ ID NO: 17 and SEQ ID NO: 18 and SEQ ID NO: 20 target the 3′,5′ and center region of U32a respectively. Additional oligo modifications may include, but are not limited to: 4′-C-hydroxymethyl-DNA (4′-CHM), 2′-0,4′-C-methylene bridged nucleic acids (2′,4′-BNAs or 2′,4′-BNA NC), G-clamp-like: Aminoethyl-Phenoxazine-dC (AP-dC), 2′-O-(2-(2-(2-aminoethoxy) ethoxy)ethyl) (2′-O-AEE), 2′-O—(N-methyl)aminopropyl (2′-O-MAP), 7-Deazapurine nucleoside analogs, 5-(hydroxymethyl)-2′-deoxycytidine (5-hm-dC), N4-methylcytosine (4-MeC), Conjugates: Cell-penetrating peptides (CPPs) conjugation, Fatty acids-TEG, Conjugation with fatty acids, such as palmitic acid, stearic acid, or myristic acid, polymer conjugation, aptamer conjugation, small molecule conjugation, antibody or antibody fragment conjugation, nanoparticle incorporation or conjugation, thioaptamers, immunomodulatory molecules and include PMO-DNA-Ps chimeras. Administration of ASO can be carried out using the various mechanisms known in the art, including naked administration and administration in pharmaceutically acceptable carriers. For example lipid carriers may be used. In general, ASO may be delivered via injection into a subject or cell to decrease expression of Rpl13a snoRNA. The injection may be an intraperitoneal injection (IP), intravenous, intramuscular, subcutaneous or intradermal. The ASO may be delivered via oral routes, transfection, electroporation, microinjection, gene gun or magnetic-assisted transfection.

In some embodiments the compositions described herein may be administered along with gene therapy. For example, ASOs can be incorporated into viral or non-viral gene delivery vectors, such as adeno-associated viruses (AAVs) or nanoparticles, and directly delivered to the target cells. Compositions described herein may also be administered together with an ex-vivo therapy or incorporated into a drug-eluting stent.

The amount of ASO administered is one effective to inhibit the expression of RP113a snoRNA. It will be appreciated that this amount will vary both with the effectiveness of the ASO delivered, the route of delivery and the nature of the carrier used. The determination of appropriate amounts for any given composition is within the skill in the art. The ASO may be delivered daily, every other day, or at another regular interval, for 1, 2, 3, 4, 5, 6, 7 or more days. Individual ASO may be delivered or multiple ASO may be combined and delivered together, for example SEQ ID NO: 33 and SEQ ID NO: 37 may be administered together, or SEQ ID NO: 33 and SEQ ID NO: 37, SEQ ID NO: 41 and SEQ ID NO: 45 may be administered together. In some embodiments, oligos that target particular snoRNA may be used alone or in combination. By way of example and not limitation, oligos that target: 32A, 33, 34, and 35A; 32A, 33, and 34; 32A, 33 and 35A; 32A, 34, and 35A; 33, 34, and 35A; 32A and 33; 32A and 34; 32A and 35A; 33 and 34; 33 and 35A; 34 and 35A may be used in combination. In some embodiments, additional snoRNAs may also be targeted. For example, snoRNA51 and snoRNA35b share sequence similarity with snoRNA32a and snoRNA35a, and so they may also be targeted. In some embodiments, snoRNA51 may be targeted in combination with or in place of U32a. In some embodiments, snoRNA35b may be targeted in combination with or in place of U35a. In some embodiments one or more, two or more, three or more or 4 or more individual snoRNA may be targeted at one time.

In another aspect, the present disclosure provides pharmaceutical compositions comprising one or more of the compositions as described herein and an appropriate carrier, excipient or diluent. The exact nature of the carrier, excipient or diluent will depend upon the desired use for the composition and may range from being suitable or acceptable for veterinary uses to being suitable or acceptable for human use. The composition may optionally include one or more additional compounds.

When used to treat or prevent a disease or symptoms of a disease, such as cardiovascular disease, atherosclerosis, coronary heart disease, cerebrovascular disease, stroke, vascular dementia, and myocardial infarction, the compositions described herein may be administered singly, as mixtures of one or more compounds or in mixture or combination with other agents (e.g., therapeutic agents) useful for treating such diseases and/or the symptoms associated with such diseases. Such agents may include, but are not limited to, antiplatelet medicines, anticoagulants, ACE inhibitors, beta blockers, calcium channel blockers, metformin, nitrates, statins, or other cholesterol-lowering, blood pressure or thrombolytic medicines, to name a few. The compounds may be administered in the form of compounds per se, or as pharmaceutical compositions comprising a compound.

Pharmaceutical compositions comprising the compound(s) may be manufactured by means of conventional mixing, dissolving, granulating, dragee-making, levigating, emulsifying, encapsulating, entrapping or lyophilization processes. The compositions may be formulated in conventional manner using one or more physiologically acceptable carriers, diluents, excipients or auxiliaries which facilitate processing of the compounds into preparations which can be used pharmaceutically. In some embodiments, the ASO may be lyophilized. Lyophilized ASO may be reconstituted in sterile water.

Pharmaceutical compositions may take a form suitable for virtually any mode of administration, including, for example, topical, ocular, oral, buccal, systemic, nasal, injection, transdermal, rectal, vaginal, etc., or a form suitable for administration by inhalation or insufflation.

For topical administration, the compound(s) may be formulated as solutions, gels, ointments, creams, suspensions, etc. as are well-known in the art. Systemic formulations include those designed for administration by injection, e.g., subcutaneous, intravenous, intramuscular, intrathecal or intraperitoneal injection, as well as those designed for transdermal, transmucosal oral or pulmonary administration.

Useful injectable preparations include sterile suspensions, solutions or emulsions of the active compound(s) in aqueous or oily vehicles. The compositions may also contain formulating agents, such as suspending, stabilizing and/or dispersing agent. The formulations for injection may be presented in unit dosage form, e.g., in ampules or in multidose containers, and may contain added preservatives. Alternatively, the injectable formulation may be provided in powder form for reconstitution with a suitable vehicle, including but not limited to sterile pyrogen free water, buffer, dextrose solution, etc., before use. To this end, the active compound(s) may be dried by any art-known technique, such as lyophilization, and reconstituted prior to use.

For transmucosal administration, penetrants appropriate to the barrier to be permeated are used in the formulation. Such penetrants are known in the art.

For oral administration, the pharmaceutical compositions may take the form of, for example, lozenges, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulfate). The tablets may be coated by methods well known in the art with, for example, sugars, films or enteric coatings.

Liquid preparations for oral administration may take the form of, for example, elixirs, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol, Cremophore™ or fractionated vegetable oils); and preservatives (e.g., methyl or propyl-p-hydroxybenzoates or sorbic acid). The preparations may also contain buffer salts, preservatives, flavoring, coloring and sweetening agents as appropriate.

Preparations for oral administration may be suitably formulated to give controlled release of the compound, as is well known. For buccal administration, the compositions may take the form of tablets or lozenges formulated in conventional manner. For rectal and vaginal routes of administration, the compound(s) may be formulated as solutions (for retention enemas) suppositories or ointments containing conventional suppository bases such as cocoa butter or other glycerides.

For nasal administration or administration by inhalation or insufflation, the compound(s) can be conveniently delivered in the form of an aerosol spray from pressurized packs or a nebulizer with the use of a suitable propellant, e.g., dichlorodifluoromethane, trichlorofluoromethane, dichlorotetrafluoroethane, fluorocarbons, carbon dioxide or other suitable gas. In the case of a pressurized aerosol, the dosage unit may be determined by providing a valve to deliver a metered amount. Capsules and cartridges for use in an inhaler or insufflator (for example capsules and cartridges comprised of gelatin) may be formulated containing a powder mix of the compound and a suitable powder base such as lactose or starch.

For ocular administration, the compound(s) may be formulated as a solution, emulsion, suspension, etc. suitable for administration to the eye. A variety of vehicles suitable for administering compounds to the eye are known in the art.

For prolonged delivery, the compound(s) can be formulated as a depot preparation for administration by implantation or intramuscular injection. The compound(s) may be formulated with suitable polymeric or hydrophobic materials (e.g., as an emulsion in an acceptable oil) or ion exchange resins, or as sparingly soluble derivatives, e.g., as a sparingly soluble salt.

Alternatively, transdermal delivery systems manufactured as an adhesive disc or patch which slowly releases the compound(s) for percutaneous absorption may be used. To this end, permeation enhancers may be used to facilitate transdermal penetration of the compound(s).

Alternatively, other pharmaceutical delivery systems may be employed. Liposomes and emulsions are well-known examples of delivery vehicles that may be used to deliver compound(s). Certain organic solvents such as dimethyl sulfoxide (DMSO) may also be employed, although usually at the cost of greater toxicity.

The pharmaceutical compositions may, if desired, be presented in a pack or dispenser device which may contain one or more unit dosage forms containing the compound(s). The pack may, for example, comprise metal or plastic foil, such as a blister pack. The pack or dispenser device may be accompanied by instructions for administration.

The compositions described herein will generally be used in an amount effective to achieve the intended result, for example in an amount effective to treat or prevent the particular disease being treated. By therapeutic benefit is meant eradication or amelioration of the underlying disorder being treated and/or eradication or amelioration of one or more of the symptoms associated with the underlying disorder such that the patient reports an improvement in feeling or condition, notwithstanding that the patient may still be afflicted with the underlying disorder. Therapeutic benefit also generally includes halting or slowing the progression of the disease, regardless of whether improvement is realized.

The amount of composition administered will depend upon a variety of factors, including, for example, the particular indication being treated, the mode of administration, whether the desired benefit is prophylactic or therapeutic, the severity of the indication being treated and the age and weight of the patient, the bioavailability of the particular composition, the conversion rate and efficiency of delivery under the selected route of administration, etc. In some embodiments, modification to the compositions described herein may alter the bioavailability or therapeutic index. For example, some compositions may be administered daily, weekly, monthly or every 2, 3, 4, 5, or 6 months, or yearly.

Determination of an effective dosage for a particular use and mode of administration is well within the capabilities of those skilled in the art. Effective dosages may be estimated initially from in vitro activity and metabolism assays. For example, an initial dosage for use in animals may be formulated to achieve a circulating blood or serum concentration of the composition that is at or above an IC₅₀ of the particular composition as measured in an in vitro assay. Calculating dosages to achieve such circulating blood or serum concentrations taking into account the bioavailability of the particular composition via the desired route of administration is well within the capabilities of skilled artisans. Initial dosages can also be estimated from in vivo data, such as animal models. Animal models useful for testing the efficacy of the active metabolites to treat or prevent the various diseases described above are well-known in the art. Animal models suitable for testing the bioavailability and/or metabolism of compositions are also well-known. Ordinarily skilled artisans can routinely adapt such information to determine dosages suitable for human administration.

Dosage amounts will typically be in the range of from about 0.0001 mg/kg/day, 0.001 mg/kg/day or 0.01 mg/kg/day to about 100 mg/kg/day, but may be higher or lower, depending upon, among other factors, the activity of the active composition, the bioavailability of the composition, its metabolism kinetics and other pharmacokinetic properties, the mode of administration and various other factors, discussed above. Dosage amount and interval may be adjusted individually to provide plasma levels which are sufficient to maintain therapeutic or prophylactic effect. For example, the compositions may be administered once per week, several times per week (e.g., every other day), once per day or multiple times per day, depending upon, among other things, the mode of administration, the specific indication being treated and the judgment of the prescribing physician. In cases of local administration or selective uptake, such as local topical administration, the effective local concentration of compositions may not be related to plasma concentration. Skilled artisans will be able to optimize effective dosages without undue experimentation.

### Methods:

In a second aspect, the present invention provides a method of preventing and/or treating cardiovascular disease in a subject comprising inhibiting a Rpl13a snoRNA, such that the cardiovascular disease is prevented and/or treated in the subject.

A “subject in need thereof” as utilized herein may refer to a subject in need of treatment for atherosclerosis or cardiovascular disease or a disease or disorder associated with atherosclerosis or cardiovascular disease. A subject in need thereof may include a subject having atherosclerosis or cardiovascular disease or a subject suspected of having atherosclerosis or cardiovascular disease that is characterized by gross abnormality visible by X-ray, computerized tomography (CT), or electrocardiogram (ECG or EKG), or PET (positron emission tomography) scan, or magnetic resonance imaging (MRI), or other method including but not limited to arteriogram, cholesterol tests, x-ray, cardiac or pharmacologic stress test intravascular ultrasound.

A “subject in need thereof” as utilized herein may also refer to a subject in need of treatment for an inflammatory disease, including a metabolic inflammatory disease and a disease associated with reactive oxygen species. By way of example and not limitation, inflammatory diseases may include: Acromegaly, Acute respiratory distress syndrome (ARDS), Addison's disease, Adrenal insufficiency, Alzheimer's disease, Amyloidosis, Anemia, Ankylosing spondylitis, Anti-glomerular basement membrane (anti-GBM) disease, Antiphospholipid syndrome, Aortitis, Asthma, Atherosclerosis, Atrial fibrillation, Autoimmune diseases, Autoimmune hepatitis, Barrett's esophagus, Becker muscular dystrophy, Behçet's disease, Behcet's disease, Berger's disease, Bladder cancer, Breast cancer, Bronchitis, Budd-Chiari syndrome, Buerger's disease, Cancer, Cardiovascular disease (CVD), Carpal tunnel syndrome, Castleman disease, Celiac disease, Charcot-Marie-Tooth disease, Cholesterol embolization syndrome, Chronic bronchitis, Chronic fatigue syndrome, Chronic inflammatory demyelinating polyneuropathy (CIDP), Chronic kidney disease, Chronic kidney disease (CKD), Chronic obstructive pulmonary disease (COPD), Chronic obstructive sleep apnea (COPD), Chronic pancreatitis, Chronic venous insufficiency, Churg-Strauss syndrome, Colorectal cancer, Crohn's disease, Cryoglobulinemia, Cushing's syndrome, Deep vein thrombosis (DVT), Depression, Dermatomyositis, Diabetic nephropathy, Diabetic neuropathy, Diabetic retinopathy, Diverticulitis, Down syndrome, Drug-induced lupus, Duchenne muscular dystrophy, Dupuytren's contracture, Eczema, Emphysema, Endometrial cancer, Eosinophilic granulomatosis with polyangiitis, Erdheim-Chester disease, Erdheim-Chester disease, Esophageal cancer, Esophagitis, Fabry disease, Facioscapulohumeral muscular dystrophy, Familial hypercholesterolemia, Fibromyalgia, Focal segmental glomerulosclerosis (FSGS), Friedreich's ataxia, Gallbladder cancer, Gallstones, Gastritis, Gastroesophageal reflux disease (GERD), Gaucher's disease, Gestational diabetes, Giant cell arteritis, Giant cell myocarditis, Glaucoma, Glomerulonephritis, Goodpasture syndrome, Gout, Graft v. Host Disease, Granulomatosis with polyangiitis, Graves' disease, Guillain-Barré syndrome, Hashimoto encephalopathy, Hashimoto's thyroiditis, Head and neck cancer, Heart attack, Heart failure, HELLP syndrome, Hemochromatosis, Hemolytic uremic syndrome, Hemolytic-uremic syndrome, Hemophilia, Hemorrhoids, Henoch-Schönlein purpura, Hepatitis, Hereditary hemochromatosis, Hidradenitis suppurativa, Host v. Graft Disease, Hunter syndrome, Hurler syndrome, Hypersensitivity vasculitis, Hypertension, Hyperthyroidism, Hypocomplementemic urticarial vasculitis syndrome (HUVS), Hypopituitarism, Hypothyroidism, Idiopathic pulmonary fibrosis (IPF), Idiopathic thrombocytopenia purpura, IgA nephropathy, IgA vasculitis, IgG4-related disease, Inflammatory bowel disease (IBD), Interstitial lung disease, Intrahepatic cholestasis of pregnancy, Irritable bowel syndrome (IBS), Juvenile idiopathic arthritis, Kawasaki disease, Kidney cancer, Kidney stones, Klinefelter syndrome, Krabbe disease, Lambert-Eaton myasthenic syndrome, Langerhans cell histiocytosis, Leukemia, Limb-girdle muscular dystrophy, Livedoid vasculopathy, Liver cancer, Lung cancer, Lupus anticoagulant syndrome, Lupus nephritis, Lymphoma, Macular degeneration, McArdle's disease, Melanoma, Membranous nephropathy, Metabolic encephalopathy, Metabolic syndrome, Microscopic polyangiitis, Migraine, Minimal change disease, Mixed connective tissue disease, Morvan syndrome, Multiple myeloma, Muscular dystrophy, Myasthenia gravis, Myocarditis, Nephrotic syndrome, Neurosarcoidosis, Niemann-Pick disease, Non-alcoholic fatty liver disease (NAFLD), Non-alcoholic steatohepatitis (NASH), Non-melanoma skin cancer, Obesity, Obesity hypoventilation syndrome, Oculopharyngeal muscular dystrophy, Osteoarthritis, Osteoporosis, Ovarian cancer, Pancreatic cancer, Pancreatic insufficiency, Panniculitis, Paraneoplastic cerebellar degeneration, Paraneoplastic syndromes, Parkinson's disease, PBC (primary biliary cholangitis), Pemphigoid, Pemphigus, Peptic ulcer disease, Peripheral artery disease (PAD), Peyronie's disease, Pneumonia, Polyarteritis nodosa, Polycystic ovary syndrome (PCOS), Polycythemia vera, Polymyalgia rheumatica, Polymyositis, Pompe's disease, Porphyria, Post-kidney transplant syndrome, Postpericardiotomy syndrome, Prader-Willi syndrome, Pre-eclampsia, Preeclampsia/eclampsia, Primary hyperoxaluria, Prostate cancer, PSC (primary sclerosing cholangitis), Psoriasis, Psoriatic arthritis, Pulmonary embolism, Pulmonary fibrosis, Rasmussen's, Raynaud's disease, Raynaud's phenomenon, Reactive arthritis, Retinopathy, Rheumatoid arthritis, Rheumatoid vasculitis, Sarcoidosis, Schnitzler syndrome, Scleroderma, Sepsis, Sickle cell disease, Sjögren-Larsson syndrome, Sjogren's syndrome, Sjögren's syndrome, Sleep apnea, Stevens-Johnson syndrome, Stroke, Systemic inflammatory response syndrome (SIRS), Systemic lupus erythematosus, Systemic lupus erythematosus (SLE), Systemic sclerosis, Systemic vasculitis, Takayasu's arteritis, Tay-Sachs disease, Temporal arteritis, Temporomandibular joint disorder (TMJ), Thalassemia, Thrombotic thrombocytopenia purpura, Thyroid cancer, Toxic epidermal necrolysis, Transplant Rejection, Tuberculosis, Turner syndrome, Type 2 diabetes, Ulcerative colitis, Uveitis, Varicose veins, Von Willebrand disease, Wegener's granulomatosis, Wilson-Mikity syndrome and Wilson's disease.

By way of example, and not limitation, reactive oxygen driven disease include, but are not limited to: Adrenal fatigue, Age-related macular degeneration (AMD), Aging, Alopecia, Alzheimer's disease, Amphetamine abuse, Amyotrophic lateral sclerosis (ALS), Androgenetic alopecia, Anemia, Angina pectoris, Ankylosing spondylitis, Anorexia nervosa, Antiphospholipid antibody syndrome (APS), Aplastic anemia, Arteriosclerosis, Asbestosis, Asthma, Atherosclerosis, Atopic dermatitis, Attention deficit hyperactivity disorder (ADHD), Autism spectrum disorder, Autism spectrum disorder (ASD), Autoimmune diseases, Barrett's esophagus, Batten disease, Bipolar disorder, Bladder cancer, Blepharitis, Brain injury, Breast cancer, Bronchitis, Burn injury, Cachexia, Cancer, Candidiasis, Cardiomyopathy, Carpal tunnel syndrome, Cataracts, Celiac disease, Central sleep apnea, Charcot-Marie-Tooth disease, Chronic fatigue syndrome, Chronic kidney disease, Chronic obstructive pulmonary disease (COPD), Chronic traumatic encephalopathy (CTE), Cirrhosis, Colitis, Colorectal cancer, Complex regional pain syndrome (CRPS), Congestive heart failure, Constrictive pericarditis, Coronary artery disease (CAD), Crohn's disease, Cushing's syndrome, Cystic fibrosis, Dementia, Depression, Diabetes mellitus, Diabetic nephropathy, Diabetic neuropathy, Diabetic retinopathy, Down syndrome, Dupuytren's, Eczema, Emphysema, Endometriosis, Epilepsy, Erectile dysfunction, Fibromyalgia, Gastric cancer, Gastritis, Gastroesophageal reflux disease (GERD), Glaucoma, Glioma, Gout, Graves' disease, Hearing loss, Heart attack, Heart failure, Hemochromatosis, Hemorrhagic stroke, Hepatitis, Herpes simplex virus (HSV) infection, Huntington's disease, Hypertension, Hypothyroidism, Inflammatory bowel disease (IBD), Insomnia, Intervertebral disc degeneration, Ischemic stroke, Kidney stones, Leukemia, Liver cancer, Liver cirrhosis, Lung cancer, Lupus, Lymphoma, Macular degeneration, Major depressive disorder, Male infertility, Melanoma, Menopause, Migraine, Mitochondrial diseases, Multiple myeloma, Multiple sclerosis, Myocardial infarction, Narcolepsy, Nasal polyps, Non-alcoholic fatty liver disease (NAFLD), Non-alcoholic steatohepatitis (NASH), Obesity, Obsessive-compulsive disorder (OCD), Osteoarthritis, Osteoporosis, Ovarian cancer, Pancreatic cancer, Parkinson's disease, Periodontal disease, Periodontitis, Peripheral arterial disease (PAD), Peripheral artery disease (PAD), Peyronie's disease, Pneumonia, Polycystic ovary syndrome (PCOS), Post-traumatic stress disorder (PTSD), Pre-eclampsia, Premature aging, Primary biliary cholangitis (PBC), Primary sclerosing cholangitis (PSC), Prostate cancer, Psoriasis, Psoriatic arthritis, Pulmonary embolism, Pulmonary fibrosis, Pulmonary hypertension, Renal failure, Restless leg syndrome, Rheumatoid arthritis, Rosacea, Sarcoidosis, Schizophrenia, Scleroderma, Sepsis, Sexual dysfunction, Sickle cell anemia, Sickle cell disease, Sinusitis, Skin cancer, Sleep apnea, Small cell lung cancer, Spinal cord injury, Stroke, Subarachnoid hemorrhage, Systemic inflammatory response syndrome (SIRS), Systemic lupus erythematos, Systemic lupus erythematosus (SLE), Temporomandibular joint disorder (TMJ), Testicular cancer, Thrombotic thrombocytopenia purpura (TTP), Tinnitus, Traumatic brain injury, Traumatic brain injury (TBI), Tuberculosis (TB), Type 1 diabetes, Type 2 diabetes, Ulcerative colitis, Uremia, Uveitis, Varicose veins, Vascular dementia, Venous insufficiency, Ventilator-induced lung injury, Vitiligo, Von Hippel-Lindau disease, Von Hippel-Lindau syndrome, Von Willebrand disease, Waldenström macroglobulinemia, Wegener's granulomatosis, Werner syndrome, Whipple's disease, Wilson disease, Wilson's disease, Wiskott-Aldrich syndrome, X-linked agammaglobulinemia, X-linked hypophosphatemia, X-linked lymphoproliferative syndrome (XLP), X-linked severe combined immunodeficiency (X-SCID), X-linked sideroblastic anemia, Yellow nail syndrome, Zellweger syndrome, Zinc deficiency and Zollinger-Ellison syndrome. The term “subject” may be used interchangeably with the terms “individual” and “patient” and includes human and non-human mammalian subjects.

As used herein, “treatment,” “therapy” and/or “therapy regimen” refer to the clinical intervention made in response to a disease, disorder or physiological condition manifested by a patient or to which a patient may be susceptible. The aim of treatment includes the alleviation or prevention of symptoms, slowing or stopping the progression or worsening of a disease, disorder, or condition and/or the remission of the disease, disorder or condition. As used herein, the terms “prevent,” “preventing,” “prevention,” “prophylactic treatment” and the like refer to reducing the probability of developing a disease, disorder or condition in a subject, who does not have, but is at risk of or susceptible to developing a disease, disorder or condition. The term “effective amount” or “therapeutically effective amount” refers to an amount sufficient to effect beneficial or desirable biological and/or clinical results.

As used herein, the terms “prevent,” “prevention,” and preventing” refer to reducing the likelihood of a particular condition or disease state (e.g., atherosclerosis) from occurring in a subject not presently experiencing or afflicted with the condition or disease state. The terms do not necessarily indicate complete or absolute prevention. “Prevention,” encompasses any administration or application of a therapeutic or technique to reduce the likelihood of a disease developing (e.g., in a mammal, including a human). Such a likelihood may be assessed for a population or for an individual.

In some embodiments, cardiovascular disease or atherosclerosis is treated or prevented. Atherosclerosis thickening or hardening of the arteries. It is caused by a buildup of plaque in the inner lining of an artery. Plaque is made up of deposits of fatty substances, cholesterol, cellular waste products, calcium, and fibrin. As it builds up in the arteries, the artery walls become thickened and stiff and can restrict blood blow to organs and tissues. Atherosclerosis is a specific type of arteriosclerosis. Atherosclerosis is the main underlying cause of cardiovascular disease (CVD). Cardiovascular disease (CVD) is a general term that describes a disease of the heart or blood vessels. CVC can also be called heart disease. There are many different types of CVD, these include, but are not limited to coronary heart disease, stroke or transient ischemic attach, peripheral arterial disease, and aortic disease.

In some embodiments the method comprises inhibiting a Rpl13a snoRNA, wherein the Rpl13a snoRNA comprises U32a, U33, U34 or U35 or combinations thereof. In some embodiments, the Rpl13a snoRNA is inhibited by an oligonucleotide, in some embodiments the oligonucleotide comprises SEQ ID NO: 1-4 or 17-84 or combinations thereof. The inhibitor of Rpl13a may include at least one of the compositions provided herein.

In a second aspect, the present invention provides a method of preventing and/or treating inflammation in a subject comprising inhibiting a Rpl13a snoRNA, such that the inflammation is prevented and/or treated in the subject. Inflammation occurs when your immune system sends out immune cells to fight a pathogen or heal an injury. The immune cells begin an inflammatory response towards the pathogen or damaged tissue. Mediators of inflammation include cytokines. Inflammatory cytokines are signaling molecules produced by activated immune cells that promote inflammation. Examples of inflammatory cytokines include, but are not limited to, interleukin-1 (IL-1 including IL-1B), IL-6, IL-12, and IL-18, tumor necrosis factor alpha (TNF-α), interferon gamma (IFNγ), and granulocyte-macrophage colony stimulating factor (GM-CSF). In some embodiments, other inflammatory markers may be affected, for example, inflammatory cells such as macrophages, reactive oxygen species (ROS), cytochrome C oxidase, or chemokines such as vascular cell adhesion protein 1 (VCAM1). In some embodiments, the Rpl13a snoRNA is inhibited by an oligonucleotide, in some embodiments the oligonucleotide comprises SEQ ID NO: 1-4 or 17-84 or combinations thereof. The inhibitor of Rpl13a for use in these methods of reducing inflammation may include at least one of the compositions provided herein.

Atherosclerosis is an inflammatory disease that involves the accumulation of lipids and inflammatory cells in medium to large arteries. Specifically, the metabolism of those lipids leads to inflammation, with associated foam cell formation arising from macrophages and vascular smooth muscle cells. In some embodiments, the methods described herein treat inflammation associated with cardiovascular disease. In some embodiments, the methods decrease IL-1B.

The present disclosure is not limited to the specific details of construction, arrangement of components, or method steps set forth herein. The compositions and methods disclosed herein are capable of being made, practiced, used, carried out and/or formed in various ways that will be apparent to one of skill in the art in light of the disclosure that follows. The phraseology and terminology used herein is for the purpose of description only and should not be regarded as limiting to the scope of the claims. Ordinal indicators, such as first, second, and third, as used in the description and the claims to refer to various structures or method steps, are not meant to be construed to indicate any specific structures or steps, or any particular order or configuration to such structures or steps. All methods described herein can be performed in any suitable order unless otherwise indicated herein or otherwise clearly contradicted by context. The use of any and all examples, or exemplary language (e.g., “such as”) provided herein, is intended merely to facilitate the disclosure and does not imply any limitation on the scope of the disclosure unless otherwise claimed. No language in the specification, and no structures shown in the drawings, should be construed as indicating that any non-claimed element is essential to the practice of the disclosed subject matter. The use herein of the terms “including,” “comprising,” or “having,” and variations thereof, is meant to encompass the elements listed thereafter and equivalents thereof, as well as additional elements. Embodiments recited as “including,” “comprising,” or “having” certain elements are also contemplated as “consisting essentially of” and “consisting of” those certain elements.

Unless otherwise specified or indicated by context, the terms “a”, “an”, and “the” mean “one or more.” For example, “a molecule” should be interpreted to mean “one or more molecules.” As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean plus or minus≤10% of the particular term and “substantially” and “significantly” will mean plus or minus >10% of the particular term.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure. Use of the word “about” to describe a particular recited amount or range of amounts is meant to indicate that values very near to the recited amount are included in that amount, such as values that could or naturally would be accounted for due to manufacturing tolerances, instrument and human error in forming measurements, and the like. All percentages referring to amounts are by weight unless indicated otherwise.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

The following examples are meant only to be illustrative and are not meant as limitations on the scope of the invention or of the appended claims.

### EXAMPLES

### Example 1

### Preliminary Results

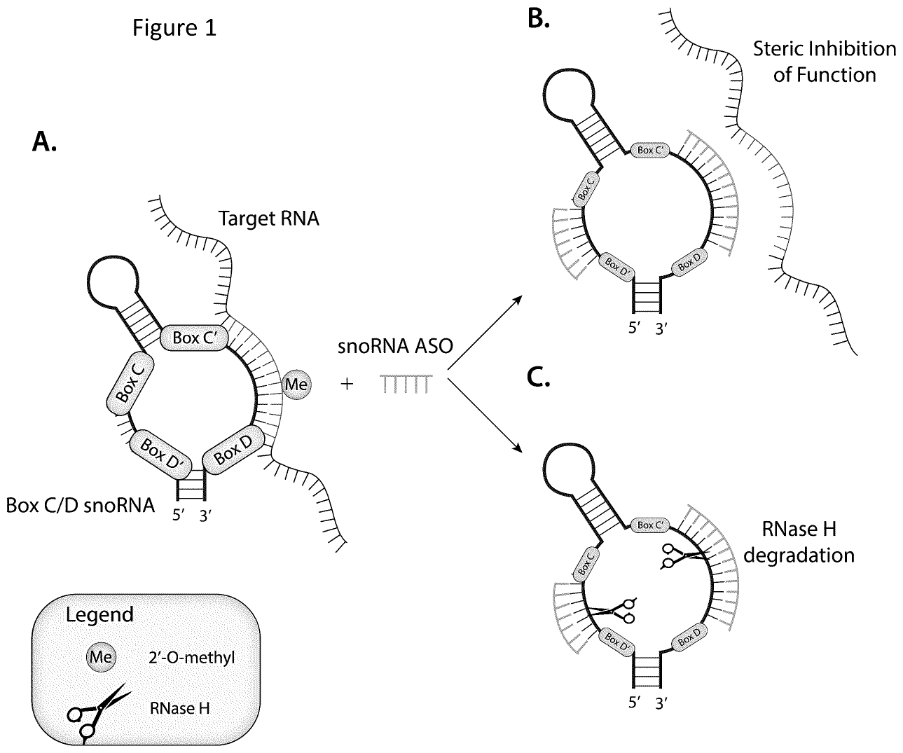

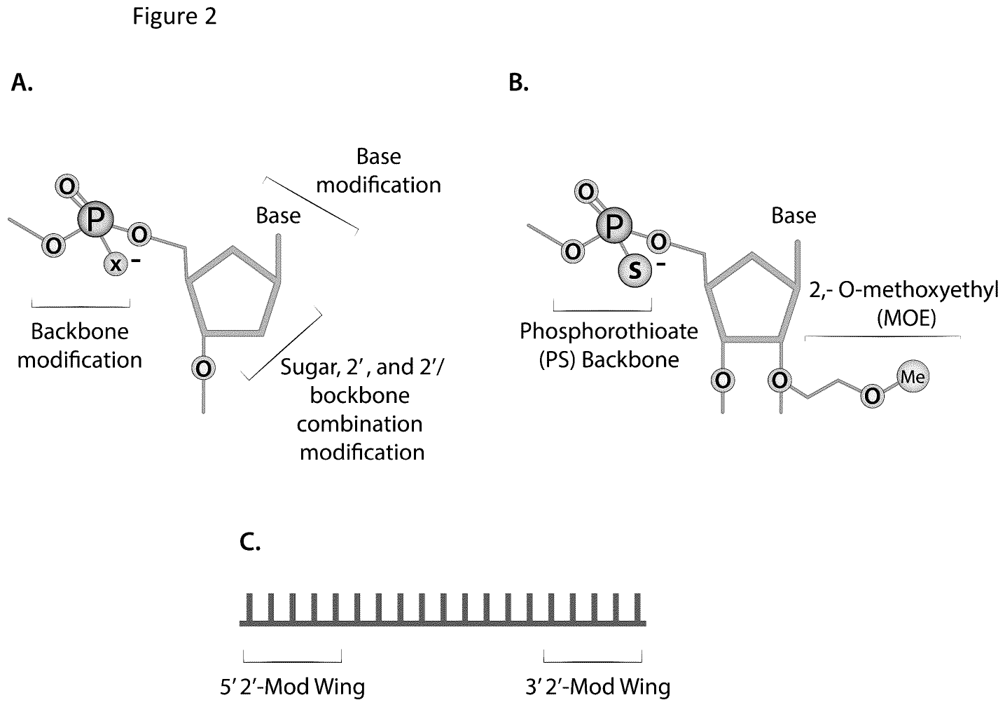

SnoRNAs augment cellular ROS levels: To identify regulators of oxidative stress, retroviral promoter trap mutagenesis was used in CHO cells. Resistance to oxidative stress in a clone of cells with Rpl13a disruption derived not from deficiency of RPL13A, but rather from deficiency of 4 snoRNAs encoded in Rpl13a introns 2, 4, 5 and 623: U32a, U33, U34, and U35a. These snoRNAs augment cellular ROS levels and oxidative stress in vitro and in vivo. These box C/D snoRNAs canonically function by guiding the methylation of target RNAs, via antisense elements on the snoRNA that hybridize with target RNA (FIG. 1A). These interactions can be inhibited by antisense oligonucleotides (ASO; FIG. 1B, C). ASOs for in vivo use typically are designed to resist nuclease-mediated degradation by incorporating chemical modifications to the ASO backbone, bases, and/or 2′-modifications (FIG. 2). The Rpl13a snoKO mouse was created by deleting the 4 Rpl13a-intronic snoRNAs listed above without affecting the expression of Rpl13a mRNA or protein. Achieved by either antisense oligonucleotides in vivo or targeted deletion, deficiency of Rpl13a snoRNAs reduces tissue ROS levels, LPS-induced hepatic oxidative stress, and insulin resistance.

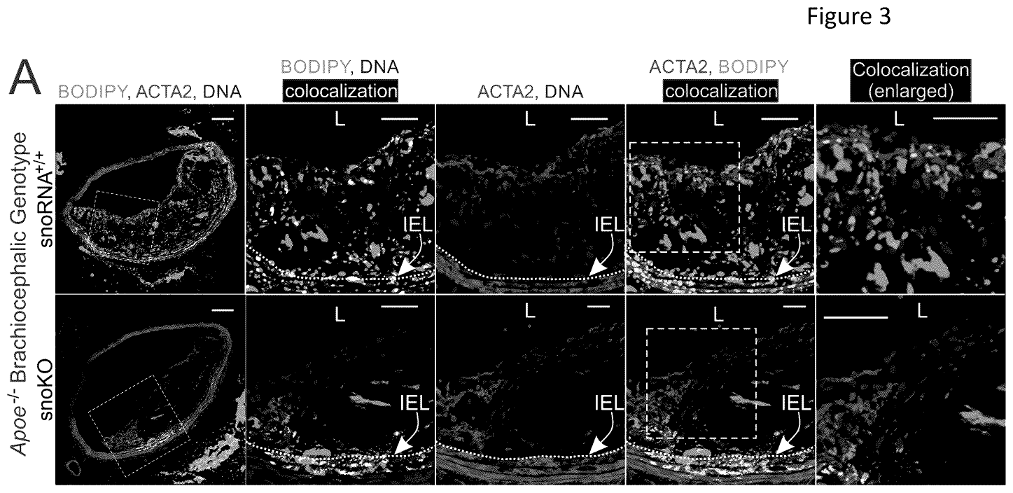

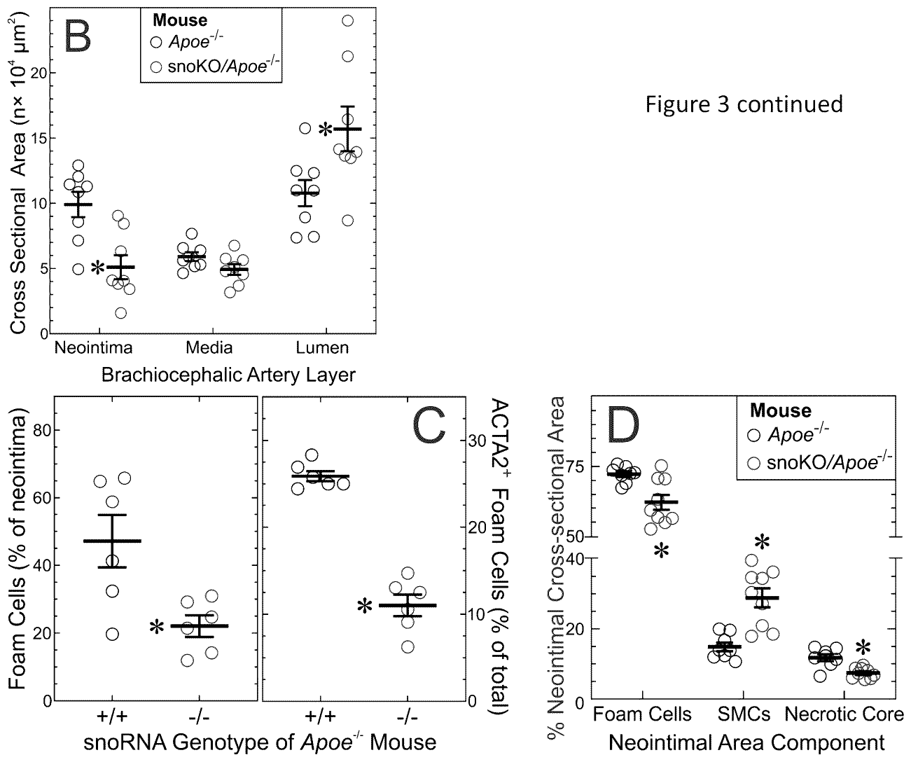

SnoRNAs increase athero: To test the role of snoRNAs in athero, we compared snoKO/Apoe−/− and Apoe−/− mice. By tail cuff manometry, snoRNA+/+ and snoKO mice had equivalent heart rates and SBPs: 475±20 v. 420±80 bpm; 133±5 v. 132±7 mm Hg, n=5/group. By CellROX Orange staining in 8-wk-old mice, ROS levels were 33±5% lower in snoKO than in snoRNA+/+ aortas (p<0.01, not shown). After 14 wk of Western diet, snoKO/Apoe−/− mice had ˜50% smaller brachiocephalic lesions and ˜50% larger lumens than snoRNA+/+ mice, despite having serum cholesterol levels3 20±4% higher than Apoe−/− mice (FIG. 3A, B). Aortic athero lesion surface area was 20% less in snoKO than in snoRNA+/+ mice (8±2% vs 10±3% of aortic surface, n=14-22/group, p=0.03). Significantly, in whole aortas from Apoe−/− mice at 8 wk of age (pre-athero) and at 22 wk (after 14 wk of Western diet), U32A, U33, U34, and U35A levels were equivalent (n=3/group). Thus, athero does not change snoRNA levels, and systemic snoRNA activity aggravates athero.

Among foam cells in Apoe−/− mouse atheromata, ˜40-70% derive from medial SMCs.29 SMC ROS promote SMC-to-foam-cell transdifferentiation, which augments athero. Do the lower ROS levels in snoKO arteries (above) correlate with less SMC-to-foam-cell transdifferentiation? To address this, SMC-derived foam cells were identified by co-localizing cholesteryl ester and ACTA2, as reported. This approach identifies fewer SMC-derived foam cells than genetic lineage tracing, but nonetheless can be used to identify SMC-derived foam cells in human athero and to compare SMC-derived foam cells across mouse genotypes. The prevalence of ACTA2+ foam cells was 60% lower in snoKO than in snoRNA+/+ brachiocephalics (FIG. 3C). These SMC-derived Mφ-like cells have reduced efferocytic function; consequently, higher SMC-derived foam cell prevalence accords with the 1.6-fold larger necrotic core of snoRNA+/+ lesions (FIG. 3D). Thus, it appears that snoRNAs not only aggravate athero but also promote SMC-to-foam cell transdifferentiation in vivo.

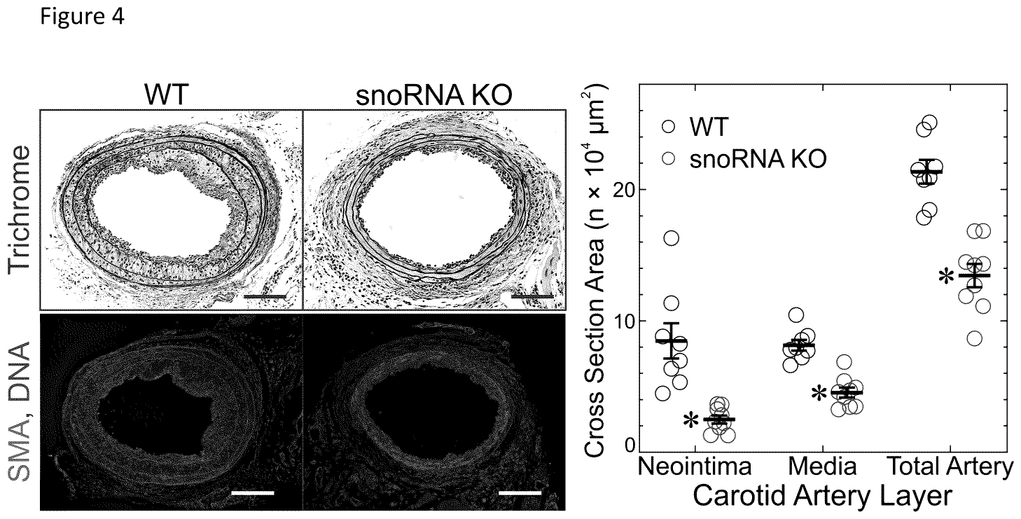

Atherogenic activity of snoRNAs in artery wall cells: To isolate possible systemic effects of snoRNAs from their vascular effects, common carotid arteries were orthotopically transplanted from snoKO vs WT mice into congenic Apoe−/− mice, as reported. These isografts develop athero that models athero in mouse aortas or brachiocephalic arteries. The athero Δ between KO and WT carotid grafts depends only on arterial wall factors, because Apoe−/− recipients of the isografts are matched for age, sex, and mass. Pre-transplant, snoKO and WT carotids were equivalent in size and histologic features (not shown). Six wk post-transplant, the athero neointima was 70% smaller in snoKO than in WT carotid grafts (FIG. 4). Concordantly, snoKO medial area and arterial cross-sectional area (reflecting MMP-dependent1 outward remodeling) were each less than WT, by 40% (FIG. 4). Thus, snoRNA deficiency in just arterial wall cells reduces athero.

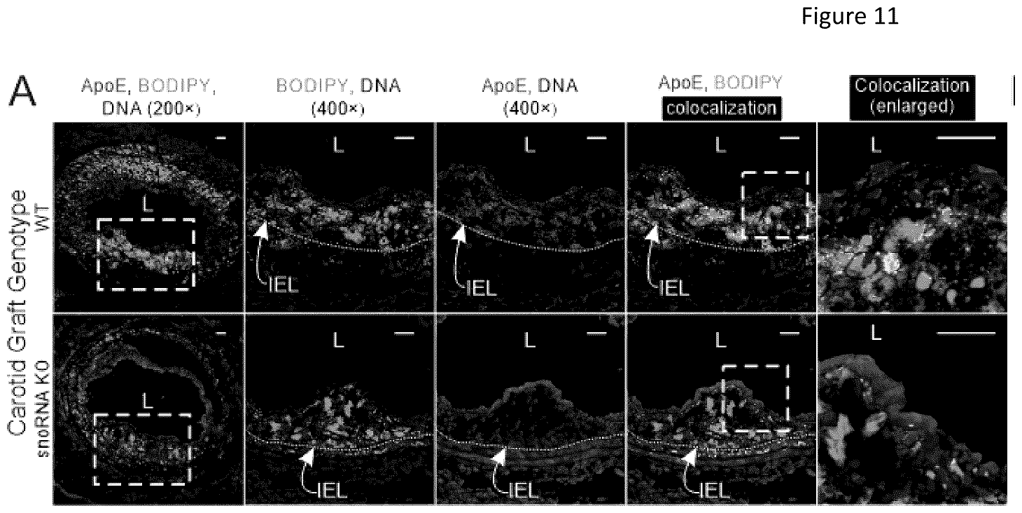

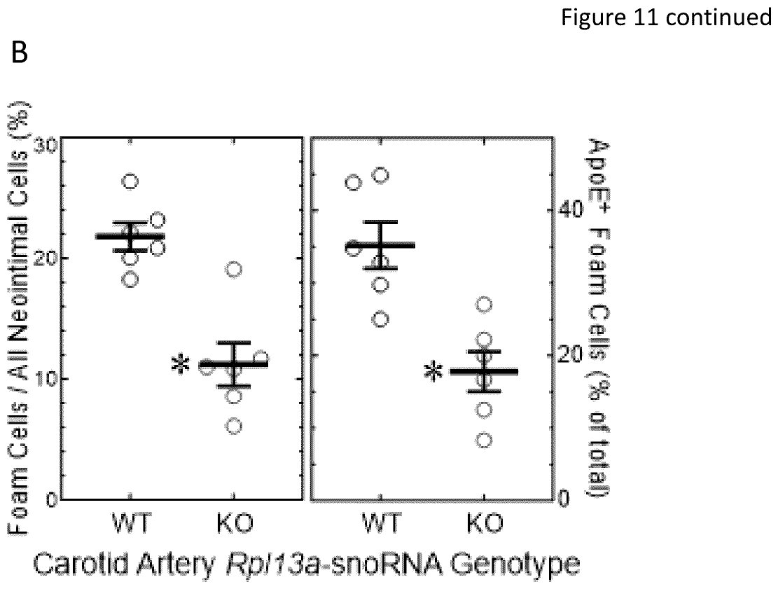

To evaluate SMC-to-foam-cell transdifferentiation in carotid grafts, we first co-localized ACTA2 with cholesteryl ester. SMC-derived foam cells were ˜40% less prevalent in snoKO than in WT carotid grafts. Next, cholesteryl ester was co-localized with apoE, which is expressed only in carotid graft cells (because the graft recipient is Apoe−/−). ApoE levels were equivalent in snoKO and WT native carotids [not shown]. ApoE+ (carotid graft-derived) foam cells were 50% less prevalent in snoKO than in WT atherosclerotic carotid grafts (FIG. 11). Thus, snoRNAs not only aggravate athero but also promote SMC-to-foam cell transdifferentiation in 2 distinct athero models.

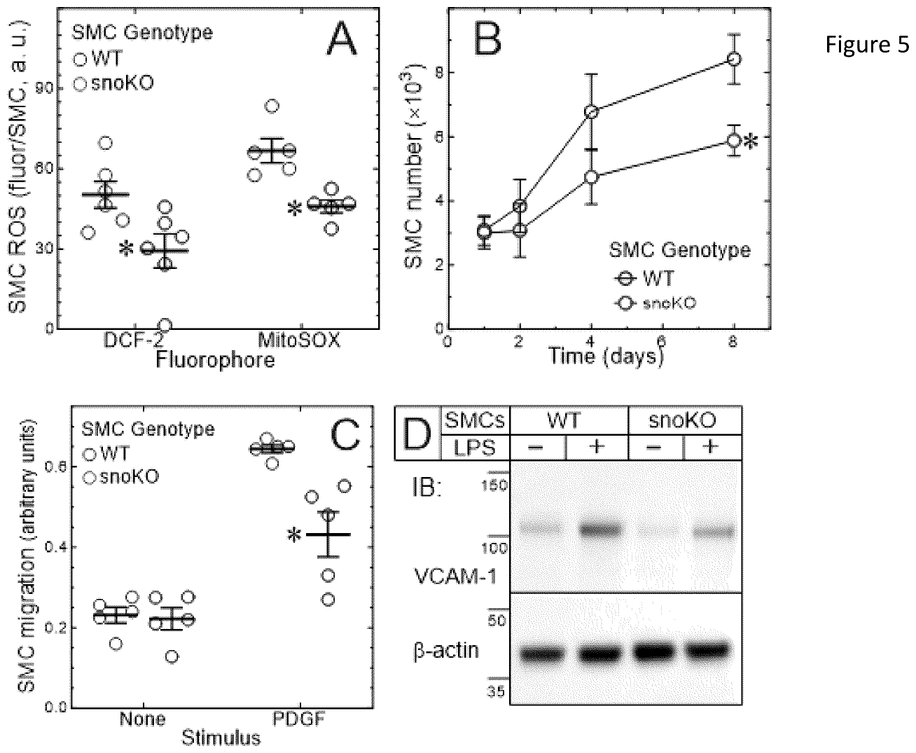

SnoRNAs augment SMC ROS levels, proliferation, migration, and inflammation: ROS regulate physiologic and pathologic SMC proliferation and migration. To determine whether snoRNAs affect these processes, primary aortic SMCs derived from C57BL/6-congenic, age-matched snoKO and WT mice were compared using ≥3 independently isolated SMC lines per genotype, as reported. SnoKO SMCs produced 30-40% less ROS than WT SMCs, assessed by MitoSOX™ Red or DCF fluorescence and flow cytometry (FIG. 5A). Concordantly, in response to 2.5% FBS, proliferation was 30% less in snoKO than in cognate WT SMCs (FIG. 5B), even though proliferation was equivalent in response to 10% FBS (not shown). Migration evoked by PDGF was also attenuated—by 30%-in snoKO SMCs (FIG. 5C). Thus, SMC ROS levels, proliferation, and migration are commensurately reduced in snoKO SMCs. It was next asked whether, with lower ROS levels than WT, snoKO SMCs have less NFκB activation (which is ROS-dependent). VCAM-1 is an NFκB-dependent gene that promotes athero. In response to the atherogenic stimuli LPS or TNF, as compared with WT SMCs, snoKO SMCs produced ˜50% less VCAM-1 protein and (transcriptionally activated) phospho-NFκB.

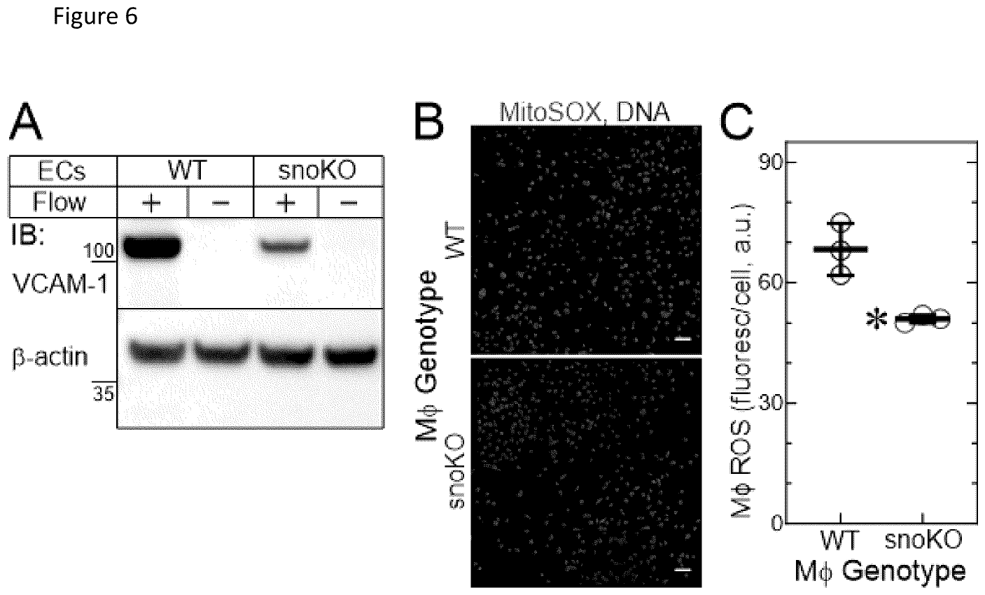

SnoRNAs augment endothelial cell (EC) inflammation: To determine whether snoRNAs promote inflammation in ECs as they do in SMCs, ECs were stimulated with (pro-inflammatory) disturbed flow, achieved with an orbital shaker. Manifest as VCAM-1 up-regulation, flow-induced EC inflammation was 3±1-fold greater in WT than in snoKO ECs (FIG. 6A, p<0.05). Thus, snoRNAs appear to promote inflammation in ECs.

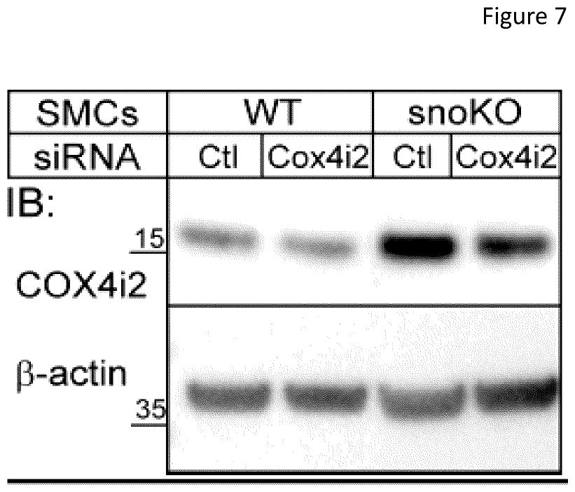

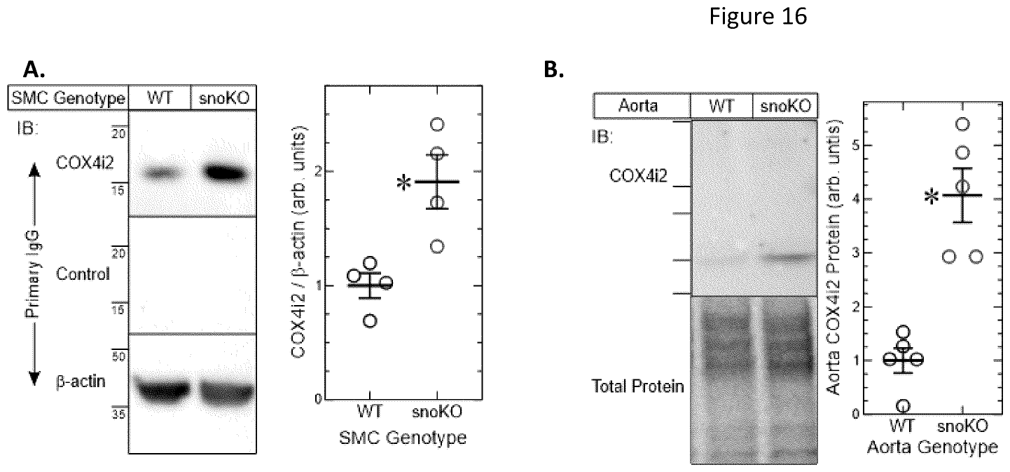

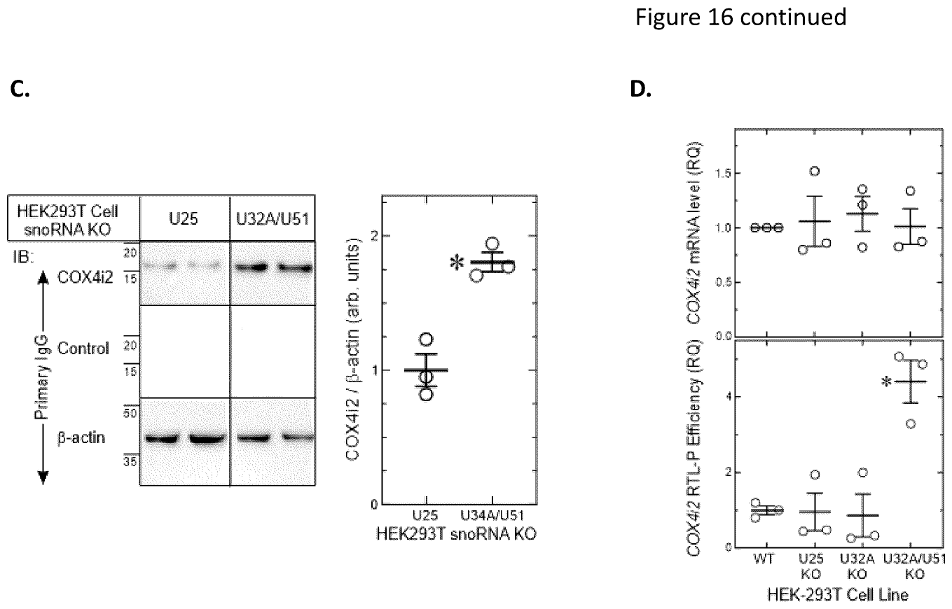

SnoRNAs augment Mφ ROS levels: To test whether snoRNA deficiency reduces mitochondrial (mito) ROS levels in Mφs, as it does in SMCs, MitoSOX Red55 was used to stain M1-polarized Mφs.11 Steady-state mito ROS levels in snoKO Mφs were 25% lower than in WT Mφs (FIG. 6B, C). Since MitoROS are required for atherogenic Mφ activity these data support studies proposed for Aim 2. Molecular mechanisms by which snoRNAs augment SMC ROS: This issue was first investigated with LC/MS/MS to compare the proteomes of snoKO and WT SMCs (Table 1). Of the 5,681 SMC proteins detected, 90 were found to distinguish snoKO from WT SMCs, with KO/WT expression ratios of ≥1.5 or ≤0.67, and nominal p values <0.05 (Table 1). This differential protein expression accords with snoKO SMC properties shown in FIG. 5: a phenotype less “activated” or inflammatory than WT (e.g., VCAM-1 [Table 1 and FIG. 5]). Perhaps because of lower ROS levels (FIG. 5), snoKO SMCs have ˜40% lower levels of proteins that protect against oxidation (e.g., HMOX-1, GST; Table 1), except carbonyl attributable to their 5.7-fold higher expression of cytochrome C oxidase subunit 4 isoform 2 (COX4i2). COX comprises 14 subunits; 11 of these are encoded by nuclear (rather than mito) genes- and therefore could be regulated by snoRNAs. COX4i1 is typically more prevalent than COX4i2, except in certain SMCs. COX4i2 is more efficient at reducing O₂ to H₂O under normoxic conditions. Consequently, when COX4i2 levels are higher and electrons are used more efficiently by mito complex IV to reduce O₂ to H₂O, there is less build-up of electrons in mito complexes I-III, less O2- production, and therefore lower cellular ROS levels.27,28,66. As compared with COX4i1, COX4i2 expression also appears to protect cells against exogenous oxidant stress. Mito-derived ROS play important roles in athero. Whether increased COX4i2 expression underlies the lower ROS levels obtained in snoKO SMCs is a key question addressed in Aim 2. Initial experiments to silence COX4i2 in SMCs demonstrate by immunoblotting that snoKO SMCs express ˜2-fold more COX4i2 than WT SMCs (3 independently-derived snoKO vs 3 WT SMC lines, FIG. 7 and FIG. 16A, p<0.05). Nevertheless, Cox4i2 mRNA levels were equivalent in WT and snoKO SMCs by qRT-PCR (data not shown). This disparity between relative mRNA and protein levels accords with our discovery that snoRNA-mediated Nm on mRNA can repress translation, and that loss of the snoRNA and mRNA Nm leads to more protein.

TABLE 1
snoKO vs. WT SMC proteome
Table 1. Whole-proteome differential
expression analysis of snoKO and WT SMCs.

| Function of | | [SMC Protein], | # of | |
|---|---|---|---|---|
| SMC Proteins | SMC Protein | snoKO/WT | peptides | p value |
| Protect | *COX412^{24, 25} | 5.7 | 3 | 0.0112 |
| against | CRB2³⁹ | 4.0 | 8 | 0.0295 |
| oxidant stress | HMOX-1 | 0.65 | 13 | 0.0223 |
| | GSTα⁴⁰ | 0.61 | 8 | 0.0423 |
| | GST omega 1⁴⁰ | 0.54 | 17 | 0.0126 |
| Contractile | smoothelin | 2.1 | 15 | 0.0112 |
| phenotype | α1-integrin | 1.9 | 18 | 0.0196 |
| markers⁴¹ | calponin-1 | 1.7 | 22 | 0.025 |
| Maintain | Paladin⁴² | 1.9 | 27 | 0.0105 |
| contractile | synaptopodin-2⁴³ | 1.7 | 4 | 0.0103 |
| phenotype | PKG1⁴⁴ | 1.6 | 8 | 0.0355 |
| | TGFβ2⁴⁵ | 1.6 | 7 | 0.0443 |
| ↑Migration, | spectrin α⁴⁶ | 0.6 | 1 | 0.0005 |
| ±Proliferation | CDK6⁴⁷ | 0.6 | 6 | 0.0151 |
| Promote | CSF-1 | 0.66 | 7 | 0.0222 |
| Inflammation | VCAM-1 | 0.54 | 41 | 0.0197 |

Six snoKO and 6 WT mice matched for age and sex were used to create 3 independent aortic SMC lines¹¹ of each genotype. SMCs were harvested at passage 3, and total protein extracts were trypsinized, labeled with tandem mass tags (TMTs) and processed for liquid chromatography/tandem mass spectrometry by the Duke Proteomics Core. Two unique TMT labels were used for each SMC line to facilitate evaluation of assay precision. We used a 1% false discovery rate. The coefficient of variation was 4.4% between technical duplicates; it was 12% and 14% for WT and snoKO SMC biological replicates, respectively. Using log₂-transformed data, we calculated KO/WT protein concentration ratios and 2-tailed p values from heteroscedastic t tests. There were 90 proteins with nominal p < 0.05 and a KO/WT ratio of ≥1.5 or ≤0.67. Proteins listed exemplify several categories into which these 90 proteins could be grouped.
*Abbreviations: CDK, cyclin-dependent kinase; COX412, cytochrome C oxidase subunit 4 isoform 2; CRB2, carbonyl reductase 2; CSF, colony-stimulating factor; GST, glutathione S-transferase; HMOX, heme oxygenase; PKG, cGMP-dependent protein kinase.

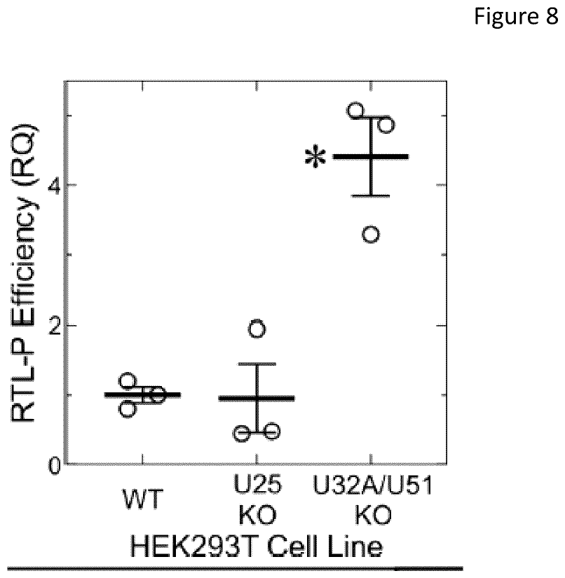

Human COX4i2 mRNA is regulated by Rpl13a-snoRNA: It was discovered that the Rpl13a snoRNA U32A interacts with Peroxidasin mRNA, and thereby promotes 2′-O-methylation (Nm) of this mRNA by fibrillarin. Do Rpl13a snoRNAs augment cellular ROS by promoting Nm of other mRNAs, thereby reducing their translation? To address this question, the inventors began by assaying COX4i2 mRNA for Nm modification. Multiple clones of (human) HEK293T cells were used in which either an irrelevant snoRNA U25 or both the Rpl13a-snoRNA U32A and U51 were knocked out, as reported. (U51's lone antisense RNA-targeting site duplicates 1 of the 2 in U32A) The mRNA from these cells was assayed for Nm of COX4i2 mRNA as reported. There was no A in transcript abundance among cell lines (FIG. 16D). However, compared with WT and U25 KO cells, cells deficient in U32A and U51 showed loss of Nm in COX4i2 mRNA (FIG. 8 and FIG. 16D). As shown in FIG. 16C, U32A/U51-DKO cells expressed 80% more COX4i2 protein than U25 KO cells. Because snoKO SMCs and whole aortic samples have normal U51 but up-regulated COX4i2 protein (FIG. 16A, B), it was inferred that U32A facilitates Nm of COX4i2 mRNA.

Mapping Nm sites on mRNA: In order to identify snoRNA-guided Nm modifications of mRNA, we have been developing methods to detect and positionally map Nm sites. Transcriptome-wide mapping for Nm exploits the lack of chemical reactivity that is imparted by the Nm modification. Nm modification at a site makes it resistant to chemical treatments of both alkaline hydrolysis and oxidation-elimination. Both of these reactions have been exploited to create RNA-seq libraries that can identify Nm sites, but only the oxidation-elimination chemistry can be efficiently used for mapping Nm sites on highly complex, low abundance mRNA.

### References for Example 1

1. Wu J H, Goswami R, Cai X, Exum S T, Huang X, Zhang L, Brian L, Premont R T, Peppel K, Freedman N J. Regulation of the platelet-derived growth factor receptor-β by G protein-coupled receptor kinase-5 in vascular smooth muscle cells involves the phosphatase Shp2. J Biol Chem 2006; 281:37758-72.
2. Wu J H, Fanaroff A C, Sharma K C, Smith L S, Brian L, Eipper B A, Mains R E, Freedman N J, Zhang L. Kalirin Promotes Neointimal Hyperplasia by Activating Rac in Smooth Muscle Cells. Arterioscler Thromb Vasc Biol 2013; 33:702-8.
3. Kim J, Zhang L, Peppel K, Wu J H, Zidar D A, Brian L, DeWire S M, Exum S T, Lefkowitz R J, Freedman N J. b-arrestins regulate atherosclerosis and neointimal hyperplasia by controlling smooth muscle cell proliferation and migration. Circ Res 2008; 103:70-9.
4. Wu J H, Zhang L, Fanaroff A C, Cai X, Sharma K C, Brian L, Exum S T, Shenoy S K, Peppel K, Freedman N J. G Protein-coupled Receptor Kinase-5 Attenuates Atherosclerosis by Regulating Receptor Tyrosine Kinases and 7-transmembrane Receptors. Arterioscler Thromb Vasc Biol 2012; 32:308-16.
5. Jean-Charles P Y, Zhang L, Wu J H, Han S O, Brian L, Freedman N J, Shenoy S K. Ubiquitin-specific protease 20 Regulates the Reciprocal Functions of β-arrestin2 in Toll-like Receptor 4-promoted NFκB Activation. J Biol Chem 2016; 291:7450-64.
6. Sundaresan M, Yu Z X, Ferrans V J, Irani K, Finkel T. Requirement for generation of H₂O₂ for platelet-derived growth factor signal transduction. Science 1995; 270:296-9.
7. Stiber J A, Wu J H, Zhang L, Nepliouev I, Zhang Z S, Bryson V G, Brian L, Bentley R C, Gordon-Weeks P R, Rosenberg P B, Freedman N J. The Actin-Binding Protein Drebrin Inhibits Neointimal Hyperplasia. Arterioscler Thromb Vasc Biol 2016; 36:984-93.
8. Zhang L, Wu J H, Huang T Q, Nepliouev I, Brian L, Zhang Z, Wertman V, Rudemiller N P, McMahon T J, Shenoy S K, Miller F J, Crowley S D, Freedman N J, Stiber J A. Drebrin regulates angiotensin I I-induced aortic remodelling. Cardiovasc Res 2018; 114:1806-15.
9. Vendrov A E, Hakim Z S, Madamanchi N R, Rojas M, Madamanchi C, Runge M S. Atherosclerosis is attenuated by limiting superoxide generation in both macrophages and vessel wall cells. Arterioscler Thromb Vasc Biol 2007; 27:2714-21.
10. Vendrov A E, Sumida A, Canugovi C, Lozhkin A, Hayami T, Madamanchi N R, Runge M S. NOXA1-dependent NADPH oxidase regulates redox signaling and phenotype of vascular smooth muscle cell during atherogenesis. Redox Biol 2019; 21:101063.
11. Jean-Charles P Y, Wu J H, Zhang L, Kaur S, Nepliouev I, Stiber J A, Brian L, Qi R, Wertman V, Shenoy S K, Freedman N J. Ubiquitin-specific Protease 20 inhibits tumor necrosis factor-triggered smooth muscle cell inflammation and attenuates atherosclerosis. Arterioscler Thromb Vasc Biol 2018; 38:2295-305.
12. Lu Y, Zhang L, Liao X, Sangwung P, Prosdocimo D A, Zhou G, Votruba A R, Brian L, Han Y J, Gao H, Wang Y, Shimizu K, Weinert-Stein K, Khrestian M, Simon D I, Freedman N J, Jain M K. Kruppel-like factor 15 is critical for vascular inflammation. J Clin Invest 2013; 123:4232-41.
13. Zhang L, Connelly J J, Peppel K, Brian L, Shah S H, Nelson S, Crosslin D R, Wang T, Allen A, Kraus W E, Gregory S G, Hauser E R, Freedman N J. Aging-related atherosclerosis is exacerbated by arterial expression of tumor necrosis factor receptor-1: evidence from mouse models and human association studies. Hum Mol Genet 2010; 19:2754-66.
14. Zhang L, Peppel K, Sivashanmugam P, Orman E S, Brian L, Exum S T, Freedman N J. Expression of tumor necrosis factor receptor-1 in arterial wall cells promotes atherosclerosis. Arterioscler Thromb Vasc Biol 2007; 27:1087-94.
15. Hennekens C H, Buring J E, Manson J E, Stampfer M, Rosner B, Cook N R, Belanger C, LaMotte F, Gaziano J M, Ridker P M, Willett W, Peto R. Lack of effect of long-term supplementation with beta carotene on the incidence of malignant neoplasms and cardiovascular disease. N Engl J Med 1996; 334:1145-9.
16. Brown B G, Zhao X Q, Chait A, Fisher L D, Cheung M C, Morse J S, Dowdy A A, Marino E K, Bolson E L, Alaupovic P, Frohlich J, Albers J J. Simvastatin and niacin, antioxidant vitamins, or the combination for the prevention of coronary disease. N Engl J Med 2001; 345:1583-92.
17. Yusuf S, Dagenais G, Pogue J, Bosch J, Sleight P. Vitamin E supplementation and cardiovascular events in high-risk patients. N Engl J Med 2000; 342:154-60.
18. Lonn E, Bosch J, Yusuf S, Sheridan P, Pogue J, Arnold J M, Ross C, Arnold A, Sleight P, Probstfield J, Dagenais G R. Effects of long-term vitamin E supplementation on cardiovascular events and cancer: a randomized controlled trial. Jama 2005; 293:1338-47.
19. Michel C I, Holley C L, Scruggs B S, Sidhu R, Brookheart R T, Listenberger L L, Behlke M A, Ory D S, Schaffer J E. Small Nucleolar RNAs U32a, U33, and U35a Are Critical Mediators of Metabolic Stress. Cell Metab 2011; 14:33-44.
20. Zhang L, Wu J H, Otto J C, Gurley S B, Hauser E R, Shenoy S K, Nagi K, Brian L, Wertman V, Mattocks N, Lawson J H, Freedman N J. Interleukin-9 mediates chronic kidney disease-dependent vein graft disease: a role for mast cells. Cardiovasc Res 2017; 113:1551-9.
21. Holley C L, Li M W, Scruggs B S, Matkovich S J, Ory D S, Schaffer J E. Cytosolic accumulation of small nucleolar RNAs (snoRNAs) is dynamically regulated by NADPH oxidase. J Biol Chem 2015; 290:11741-8.
22. Lee J, Harris A N, Holley C L, Mahadevan J, Pyles K D, Lavagnino Z, Scherrer D E, Fujiwara H, Sidhu R, Zhang J, Huang S C-c, Piston D W, Remedi M S, Urano F, Ory D S, Schaffer J E. Rpl13a small nucleolar RNAs regulate systemic glucose metabolism. J Clin Invest 2016; 126:4616-25.
23. Elliott B A, Ho H T, Ranganathan S V, Vangaveti S, Ilkayeva O, Abou Assi H, Choi A K, Agris P F, Holley C L. Modification of messenger RNA by 2′-O-methylation regulates gene expression in vivo. Nat Commun 2019; 10:3401.
24. Schiffer T A, Peleli M, Sundqvist M L, Ekblom B, Lundberg J O, Weitzberg E, Larsen F J. Control of human energy expenditure by cytochrome c oxidase subunit IV-2. Am J Physiol Cell Physiol 2016; 311: C452-61.
25. Pajuelo Reguera D, Čunátová K, Vrbacký M, Pecinová A, Houštěk J, Mráček T, Pecina P. Cytochrome c Oxidase Subunit 4 Isoform Exchange Results in Modulation of Oxygen Affinity. Cells 2020; 9.
26. Wang Y, Dubland J A, Allahverdian S, Asonye E, Sahin B, Erh Jaw J, Sin D D, Seidman M A, Leeper N J, Francis G A. Smooth Muscle Cells Contribute the Majority of Foam Cells in ApoE (Apolipoprotein E)-Deficient Mouse Atherosclerosis. Arterioscler Thromb Vasc Biol 2019; 39:876-87.
27. Schriner S E, Linford N J, Martin G M, Treuting P, Ogburn C E, Emond M, Coskun P E, Ladiges W, Wolf N, Van Remmen H, Wallace D C, Rabinovitch P S. Extension of murine life span by overexpression of catalase targeted to mitochondria. Science 2005; 308:1909-11.
28. Zhu Y, Pirnie S P, Carmichael G G. High-throughput and site-specific identification of 2′-O-methylation sites using ribose oxidation sequencing (RibOxi-seq). RNA (New York, N Y) 2017; 23:1303-14.
29. Kudla G, Granneman S, Hahn D, Beggs J D, Tollervey D. Cross-linking, ligation, and sequencing of hybrids reveals RNA-RNA interactions in yeast. Proc Natl Acad Sci USA 2011; 108:10010-5.
30. Travis A J, Moody J, Helwak A, Tollervey D, Kudla G. Hyb: a bioinformatics pipeline for the analysis of CLASH (crosslinking, ligation and sequencing of hybrids) data. Methods 2014; 65:263-73.
31. Barry-Lane P A, Patterson C, van der Merwe M, Hu Z, Holland S M, Yeh E T, Runge M S. p47phox is required for atherosclerotic lesion progression in ApoE (−/−) mice. J Clin Invest 2001; 108:1513-22.
32. Judkins C P, Diep H, Broughton B R, Mast A E, Hooker E U, Miller A A, Selemidis S, Dusting G J, Sobey C G, Drummond G R. Direct evidence of a role for Nox2 in superoxide production, reduced nitric oxide bioavailability, and early atherosclerotic plaque formation in ApoE−/− mice. Am J Physiol Heart Circ Physiol 2010; 298:H24-32.
33. Violi F, Carnevale R, Loffredo L, Pignatelli P, Gallin J I. NADPH Oxidase-2 and Atherothrombosis: Insight From Chronic Granulomatous Disease. Arterioscler Thromb Vasc Biol 2017; 37:218-25.
34. Dinauer M C, Deck M B, Unanue E R. Mice lacking reduced nicotinamide adenine dinucleotide phosphate oxidase activity show increased susceptibility to early infection with Listeria monocytogenes. J Immunol 1997; 158:5581-3.
35. Myers J T, Tsang A W, Swanson J A. Localized reactive oxygen and nitrogen intermediates inhibit escape of Listeria monocytogenes from vacuoles in activated macrophages. J Immunol 2003; 171:5447-53.
36. Dai Q, Moshitch-Moshkovitz S, Han D, Kol N, Amariglio N, Rechavi G, Dominissini D, He C. Nm-seq maps 2′-O-methylation sites in human mRNA with base precision. Nat Methods 2017; 14:695-8.
37. Rimer J M, Lee J, Holley C L, Crowder R J, Chen D L, Hanson P I, Ory D S, Schaffer J E. Long-range function of secreted small nucleolar RNAs that direct 2′-O-methylation. J Biol Chem 2018; 293:13284-96.
38. Timón-Gómez A, Nývltová E, Abriata L A, Vila A J, Hosler J, Barrientos A. Mitochondrial cytochrome c oxidase biogenesis: Recent developments. Semin Cell Dev Biol 2018; 76:163-78.
39. Nakanishi M, Deyashiki Y, Ohshima K, Hara A. Cloning, expression and tissue distribution of mouse tetrameric carbonyl reductase. Identity with an adipocyte 27-kDa protein. Eur J Biochem 1995; 228:381-7.
40. Hayes J D, Flanagan J U, Jowsey I R. Glutathione transferases. Annu Rev Pharmacol Toxicol 2005; 45:51-88.
41. Alexander M R, Owens G K. Epigenetic control of smooth muscle cell differentiation and phenotypic switching in vascular development and disease. Annu Rev Physiol 2012; 74:13-40.
42. Jin L. The actin associated protein palladin in smooth muscle and in the development of diseases of the cardiovasculature and in cancer. J Muscle Res Cell Motil 2011; 32:7-17.
43. Turczynska K M, Sward K, Hien T T, Wohlfahrt J, Mattisson I Y, Ekman M, Nilsson J, Sjogren J, Murugesan V, Hultgardh-Nilsson A, Cidad P, Hellstrand P, Perez-Garcia M T, Albinsson S. Regulation of smooth muscle dystrophin and synaptopodin 2 expression by actin polymerization and vascular injury. Arterioscler Thromb Vasc Biol 2015; 35:1489-97.
44. Choi S, Park M, Kim J, Park W, Kim S, Lee D K, Hwang J Y, Choe J, Won M H, Ryoo S, Ha K S, Kwon Y G, Kim Y M. TNF-alpha elicits phenotypic and functional alterations of vascular smooth muscle cells by miR-155-5p-dependent down-regulation of cGMP-dependent kinase 1. J Biol Chem 2018; 293:14812-22.
45. Ardila D C, Tamimi E, Danford F L, Haskett D G, Kellar R S, Doetschman T, Vande Geest J P. TGFbeta2 differentially modulates smooth muscle cell proliferation and migration in electrospun gelatin-fibrinogen constructs. Biomaterials 2015; 37:164-73.
46. Silverman-Gavrila R V, Silverman-Gavrila L B, Bilal K H, Bendeck M P. Spectrin alpha is important for rear polarization of the microtubule organizing center during migration and spindle pole assembly during division of neointimal smooth muscle cells. Cytoskeleton (Hoboken) 2015; 72:157-70.
47. Kundumani-Sridharan V, Singh N K, Kumar S, Gadepalli R, Rao G N. Nuclear factor of activated T cells cl mediates p21-activated kinase 1 activation in the modulation of chemokine-induced human aortic smooth muscle cell F-actin stress fiber formation, migration, and proliferation and injury-induced vascular wall remodeling. J Biol Chem 2013; 288:22150-62.
48. Huttemann M, Lee I, Gao X, Pecina P, Pecinova A, Liu J, Aras S, Sommer N, Sanderson T H, Tost M, Neff F, Aguilar-Pimentel J A, Becker L, Naton B, Rathkolb B, Rozman J, Favor J, Hans W, Prehn C, Puk O, Schrewe A, Sun M, Hofler H, Adamski J, Bekeredjian R, Graw J, Adler T, Busch D H, Klingenspor M, Klopstock T, Ollert M, Wolf E, Fuchs H, Gailus-Durner V, Hrabe de Angelis M, Weissmann N, Doan J W, Bassett D J, Grossman L I. Cytochrome c oxidase subunit 4 isoform 2-knockout mice show reduced enzyme activity, airway hyporeactivity, and lung pathology. Faseb j 2012; 26:3916-30.
49. Dai D F, Johnson S C, Villarin J J, Chin M T, Nieves-Cintron M, Chen T, Marcinek D J, Dorn G W, Kang Y J, Prolla Ta, Santana L F, Rabinovitch P S. Mitochondrial oxidative stress mediates angiotensin I I-induced cardiac hypertrophy and gaq overexpression-induced heart failure. Circ Res 2011; 108:837-46.
50. Wang Y, Wang G Z, Rabinovitch P S, Tabas I. Macrophage mitochondrial oxidative stress promotes atherosclerosis and nuclear factor-κB-mediated inflammation in macrophages. Circ Res 2014; 114:421-33.
51. Kehr S, Bartschat S, Stadler P F, Tafer H. PLEXY: efficient target prediction for box C/D snoRNAs. Bioinformatics 2011; 27:279-80.
52. Busch A, Richter A S, Backofen R. IntaRNA: efficient prediction of bacterial sRNA targets incorporating target site accessibility and seed regions. Bioinformatics 2008; 24:2849-56.
53. Ballinger S W, Patterson C, Knight-Lozano C A, Burow D L, Conklin C A, Hu Z, Reuf J, Horaist C, Lebovitz R, Hunter G C, McIntyre K, Runge M S. Mitochondrial integrity and function in atherogenesis. Circulation 2002; 106:544-9.
54. Allahverdian S, Chehroudi A C, McManus B M, Abraham T, Francis G A. Contribution of intimal smooth muscle cells to cholesterol accumulation and macrophage-like cells in human atherosclerosis. Circulation 2014; 129:1551-9.
55. Vengrenyuk Y, Nishi H, Long X, Ouimet M, Savji N, Martinez F O, Cassella C P, Moore K J, Ramsey S A, Miano J M, Fisher E A. Cholesterol loading reprograms the microRNA-143/145-myocardin axis to convert aortic smooth muscle cells to a dysfunctional macrophage-like phenotype. Arterioscler Thromb Vasc Biol 2015; 35:535-46.
56. Frontini M J, O'Neil C, Sawyez C, Chan B M, Huff M W, Pickering J G. Lipid incorporation inhibits Src-dependent assembly of fibronectin and type I collagen by vascular smooth muscle cells. Circ Res 2009; 104:832-41.
57. Shankman L S, Gomez D, Cherepanova O A, Salmon M, Alencar G F, Haskins R M, Swiatlowska P, Newman A A, Greene E S, Straub A C, Isakson B, Randolph G J, Owens G K. KLF4-dependent phenotypic modulation of smooth muscle cells has a key role in atherosclerotic plaque pathogenesis. Nat Med 2015; 21:628-37.
58. Endres R, Luz A, Schulze H, Neubauer H, Fütterer A, Holland S M, Wagner H, Pfeffer K. Listeriosis in p47 (phox−/−) and TRp55−/− mice: protection despite absence of ROI and susceptibility despite presence of RNI. Immunity 1997; 7:419-32.
59. Herb M, Gluschko A, Wiegmann K, Farid A, Wolf A, Utermohlen O, Krut O, Krönke M, Schramm M. Mitochondrial reactive oxygen species enable proinflammatory signaling through disulfide linkage of NEMO. Sci Signal 2019; 12.
60. Nakashima Y, Raines E W, Plump A S, Breslow J L, Ross R. Upregulation of VCAM-1 and ICAM-1 at atherosclerosis-prone sites on the endothelium in the ApoE-deficient mouse. Arterioscler Thromb Vasc Biol 1998; 18:842-51.
61. Daugherty A, Tall A R, Daemen M, Falk E, Fisher E A, Garcia-Cardena G, Lusis A J, Owens A P, 3rd, Rosenfeld M E, Virmani R. Recommendation on Design, Execution, and Reporting of Animal Atherosclerosis Studies: A Scientific Statement From the American Heart Association. Arterioscler Thromb Vasc Biol 2017; 37: e131-e57.
62. Palinski W, Hörkkö S, Miller E, Steinbrecher U P, Powell H C, Curtiss L K, Witztum J L. Cloning of monoclonal autoantibodies to epitopes of oxidized lipoproteins from apolipoprotein E-deficient mice. Demonstration of epitopes of oxidized low density lipoprotein in human plasma. J Clin Invest 1996; 98:800-14.
63. Sakurai H, Chiba H, Miyoshi H, Sugita T, Toriumi W. IkappaB kinases phosphorylate N F-kappaB p65 subunit on serine 536 in the transactivation domain. J Biol Chem 1999; 274:30353-6.
64. Neish A S, Williams A J, Palmer H J, Whitley M Z, Collins T. Functional analysis of the human vascular cell adhesion molecule 1 promoter. J Exp Med 1992; 176:1583-93.
65. Salic A, Mitchison T J. A chemical method for fast and sensitive detection of DNA synthesis in vivo. Proc Natl Acad Sci USA 2008; 105:2415-20.
66. Galkina E, Kadl A, Sanders J, Varughese D, Sarembock I J, Ley K. Lymphocyte recruitment into the aortic wall before and during development of atherosclerosis is partially L-selectin dependent. J Exp Med 2006; 203:1273-82.
67. Zhang L, Sivashanmugam P, Wu J H, Brian L, Exum S T, Freedman N J, Peppel K. Tumor necrosis factor receptor-2 signaling attenuates vein graft neointima formation by promoting endothelial recovery. Arterioscler Thromb Vasc Biol 2008; 28:284-9.
68. Yurdagul A, Jr., Doran A C, Cai B, Fredman G, Tabas I A. Mechanisms and Consequences of Defective Efferocytosis in Atherosclerosis. Front Cardiovasc Med 2017; 4:86.
69. Zhang L, Freedman N J, Brian L, Peppel K. Graft-extrinsic cells predominate in vein graft arterialization. Arterioscler Thromb Vasc Biol 2004; 24:470-6.
70. Martinez del Hoyo G, Ramirez-Huesca M, Levy S, Boucheix C, Rubinstein E, Minguito de la Escalera M, Gonzalez-Cintado L, Ardavin C, Veiga E, Yanez-Mo M, Sanchez-Madrid F. CD81 controls immunity to Listeria infection through rac-dependent inhibition of proinflammatory mediator release and activation of cytotoxic T cells. J Immunol 2015; 194:6090-101.
71. Porturas T P, Sun H, Buchlis G, Lou Y, Liang X, Cathopoulis T, Fayngerts S, Johnson D S, Wang Z, Chen Y H. Crucial roles of TNFAIP8 protein in regulating apoptosis and Listeria infection. J Immunol 2015; 194:5743-50.
72. Cai X, Freedman N J. New therapeutic possibilities for vein graft disease in the post-edifoligide era. Future Cardiol 2006; 2:493-501.
73. Zhang X, Edwards J P, Mosser D M. The expression of exogenous genes in macrophages: obstacles and opportunities. Methods Mol Biol 2009; 531:123-43.
74. Freeman M, Ekkel Y, Rohrer L, Penman M, Freedman N J, Chisolm G M, Krieger M. Expression of type I and type I I bovine scavenger receptors in Chinese hamster ovary cells: lipid droplet accumulation and nonreciprocal cross competition by acetylated and oxidized low density lipoprotein. Proc Natl Acad Sci USA 1991; 88:4931-5.
75. Wu J H, Peppel K, Nelson C D, Lin F T, Kohout T A, Miller W E, Exum S T, Freedman N J. The adaptor protein b-arrestin2 enhances endocytosis of the low density lipoprotein receptor. J Biol Chem 2003; 278:44238-45.
76. Arthur J S, Ley S C. Mitogen-activated protein kinases in innate immunity. Nat Rev Immunol 2013; 13:679-92.
77. Rong J X, Shapiro M, Trogan E, Fisher E A. Transdifferentiation of mouse aortic smooth muscle cells to a macrophage-like state after cholesterol loading. Proc Natl Acad Sci USA 2003; 100:13531-6.
78. Liu Y, Sinha S, McDonald O G, Shang Y, Hoofnagle M H, Owens G K. Kruppel-like factor 4 abrogates myocardin-induced activation of smooth muscle gene expression. J Biol Chem 2005; 280:9719-27.
79. Stiber J A, Tabatabaei N, Hawkins A F, Hawke T, Worley P F, Williams R S, Rosenberg P. Homer modulates NFAT-dependent signaling during muscle differentiation. Dev Biol 2005; 287:213-24.
80. Gokhale N S, McIntyre A B R, Mattocks M D, Holley C L, Lazear H M, Mason C E, Horner S M. Altered m (6) A Modification of Specific Cellular Transcripts Affects Flaviviridae Infection. Mol Cell 2020; 77:542-55.e8.
81. Helwak A, Tollervey D. Identification of miRNA-Target RNA Interactions Using CLASH. Methods Mol Biol 2016; 1358:229-51.
82. Helwak A, Kudla G, Dudnakova T, Tollervey D. Mapping the human miRNA interactome by CLASH reveals frequent noncanonical binding. Cell 2013; 153:654-65.
83. Urdaneta E C, Vieira-Vieira C H, Hick T, Wessels H H, Figini D, Moschall R, Medenbach J, Ohler U, Granneman S, Selbach M, Beckmann B M. Purification of cross-linked RNA-protein complexes by phenol-toluol extraction. Nat Commun 2019; 10:990.
84. Fukuda R, Zhang H, Kim J W, Shimoda L, Dang C V, Semenza G L. HIF-1 regulates cytochrome oxidase subunits to optimize efficiency of respiration in hypoxic cells. Cell 2007; 129:111-22.
85. Cai Z, Cao C, Ji L, Ye R, Wang D, Xia C, Wang S, Du Z, Hu N, Yu X, Chen J, Wang L, Yang X, He S, Xue Y. RIC-seq for global in situ profiling of RNA-RNA spatial interactions. Nature 2020; e-pub ahead of print.

### Example 2: Use of Rpl13a snoRNAs in Cardiovascular Disease, Vascular Injury and Associated Inflammation

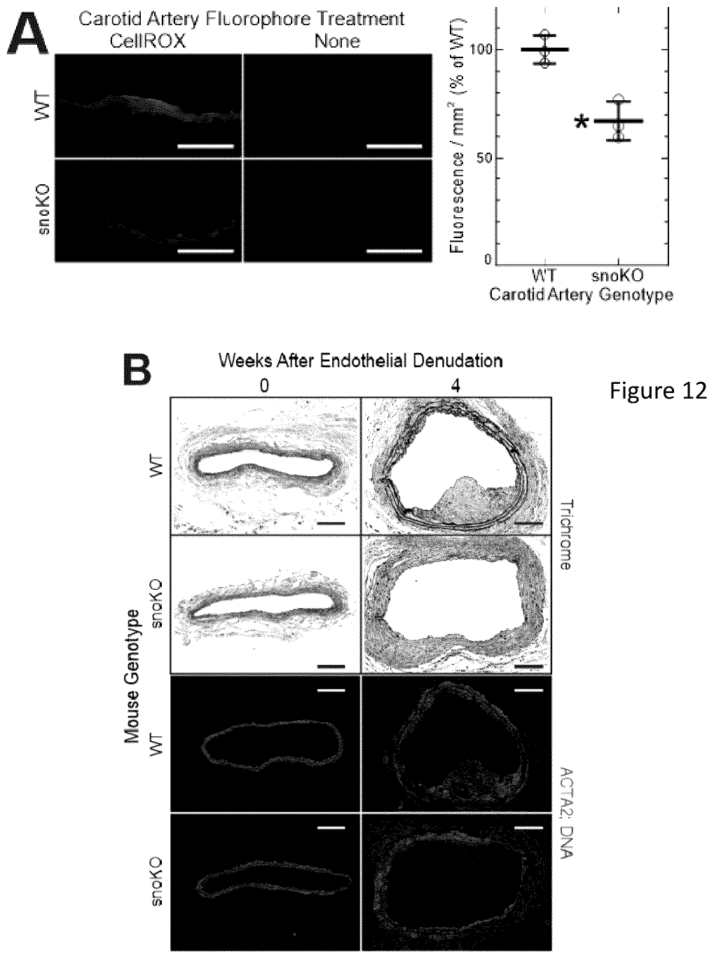

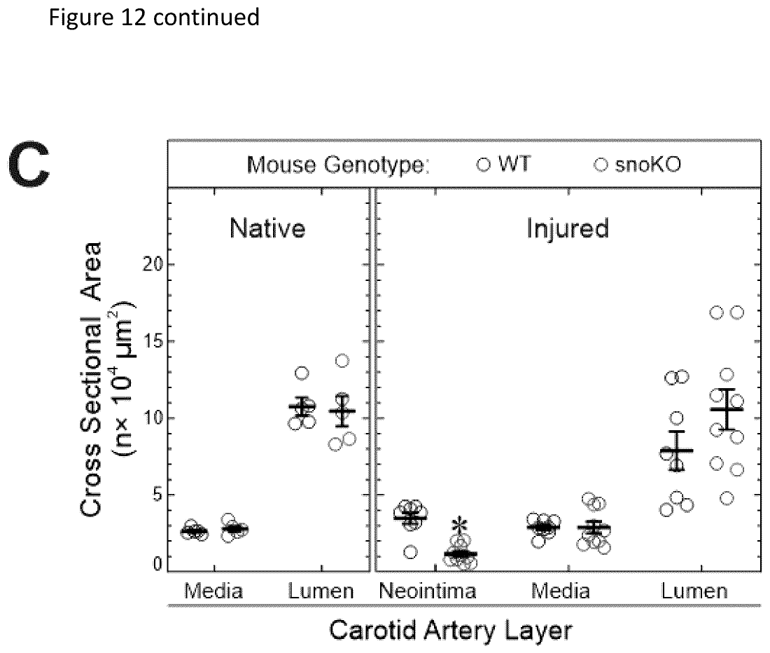

Since 2011, the Rpl13a snoRNAs are known to be critical mediators of metabolic stress, especially in response to saturated fatty acids but also in the setting of sterile inflammation. In genetically engineered mice that lack the Rpl13a snoRNAs (snoKO) but normally express the co-transcribed RPL13A protein are also protected from developing diabetes. These snoRNAs are known to guide post-transcriptional modification of rRNA via 5′- and 3′-antisense elements (ASE), recruiting a complex that modifies the rRNA with site-specific 2′-O-methylation (this is the canonical function of snoRNAs in the “box C/D” group). However, haploinsufficiency with 50% loss of these snoRNAs also protects cells from metabolic stress without altering rRNA modification, suggesting an alternative mechanism. We have recently shown that at least one of the Rpl13a snoRNAs can also guide mRNA modification, mRNA abundance and translation, and here again, the physiologic effect is seen in Rpl13a snoRNA haploinsufficiency. Although the mechanisms linking these snoRNAs to their protective metabolic phenotypes are unclear, a consistent finding is that the loss or reduction of Rpl13a snoRNA expression is associated with reduced ROS and oxidative stress. In fact, we consistently find that loss of the Rpl13a snoRNAs results in a lower levels of ROS at both the cellular/tissue level and in mitochondria (FIG. 12A).

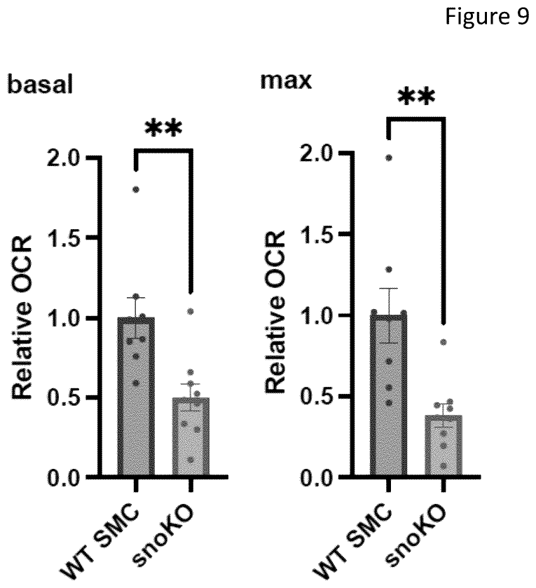

Our emerging data suggests that the likely mechanism for this ROS phenotype is at the level of mitochondrial metabolism, which is a key source of ROS. Specifically, the Rpl13a snoRNAs promote mitochondrial oxygen consumption at both basal and maximal rates (FIG. 9). Thus, we hypothesize that the loss of Rpl13a snoRNAs limits the ability of mitochondria to consume excess substrate, thereby limiting mitochondrial ROS production and preventing metabolic oxidative stress and inflammation. This upstream metabolic mechanism may be critical, as we note that efforts to treat cardiovascular disease by directly targeting ROS or inflammation have failed or been problematic (as with canakinumab). In more than 8y of experience, we have found that Rpl13a snoRNA KO mice reach adulthood without increased risk of infection, suggesting that these RNAs are a safe target for therapeutic development. We are directly testing the impact of these snoRNAs on immunity and working to discover the specific mechanisms by which they regulate mitochondrial metabolism in other work.

Metabolic stress, including ROS production, is a key feature of athero, so we have tested whether genetic loss of Rpl13a snoRNAs is beneficial in mouse models. Specifically, Apoe knockout (Apoe−/−) mice fed HFD have extremely high LDL-C (>800 mg/dL) and they develop athero either without further perturbation (14 wks), or at an accelerated rate in the setting of wire injury (6 wks) or carotid transplant (6 wks). When Apoe−/− mice were crossed with snoKO mice in our studies, the snoKO/Apoe−/− mice have significantly decreased levels of ROS in the aorta, carotid arteries, isolated aortic SMCs, and activated bone marrow-derived Mφ (FIG. 12 and not shown). Compared with Apoe−/− controls, snoKO/Apoe−/− mice had significantly smaller athero lesions, less foam cell formation, and less SMC-to-foam-cell transdifferentiation (FIG. 3 and not shown). SnoKO/Apoe−/− mice had ˜50% smaller brachiocephalic artery (BCA) lesions and ˜50% larger lumens than control mice (FIG. 3). Athero lesion crosssections in snoKO/Apoe−/− mice had less foam cell-positive area, more ACTA2⁺ smooth muscle cells, and 40% less necrotic core area than in control lesions. To isolate possible systemic effects of snoRNAs from their vascular effects, we orthotopically transplanted common carotid arteries from snoKO vs WT mice into Apoe−/− mice.

In this model, the athero difference between snoKO and WT carotid grafts depends only on arterial wall factors, because Apoe−/− recipients of the isografts are matched for age, sex, and mass, and they express normal amounts of Rpl13a snoRNAs. Pre-transplant, snoKO and WT carotids were equivalent in size and histologic features (not shown). Six weeks post-transplant, the athero neointima was 70% smaller in snoKO than WT carotid grafts (FIGS. 4A and B). Concordantly, snoKO medial area and arterial cross-sectional area were each less than WT, by 40%.

These results suggest that the effect of the Rpl13a snoRNAs on the development of athero are at least in part due to expression of the snoRNAs in the artery itself, as opposed to circulating immune cells from the recipient bone marrow. At the cellular level, ApoE+ (carotid graft-derived) foam cells (identified as we reported 19) were 50% less prevalent in snoKO than in WT atherosclerotic carotid grafts (not shown). To further evaluate SMC-to-foam-cell transdifferentiation in the carotid grafts, we co-localized ACTA2⁺ cells (smooth muscle origin) with cholesteryl ester. SMC-derived foam cells were ˜40% less prevalent in snoKO than in WT carotid grafts.

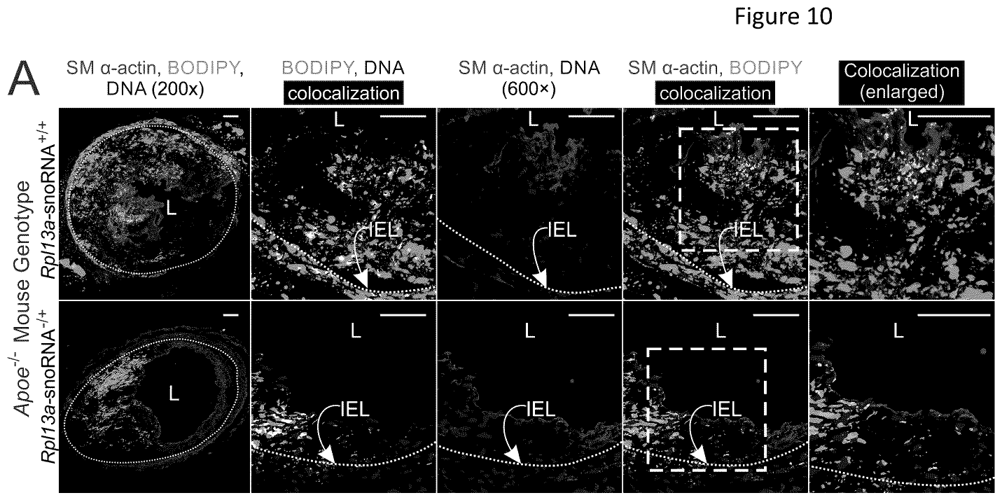

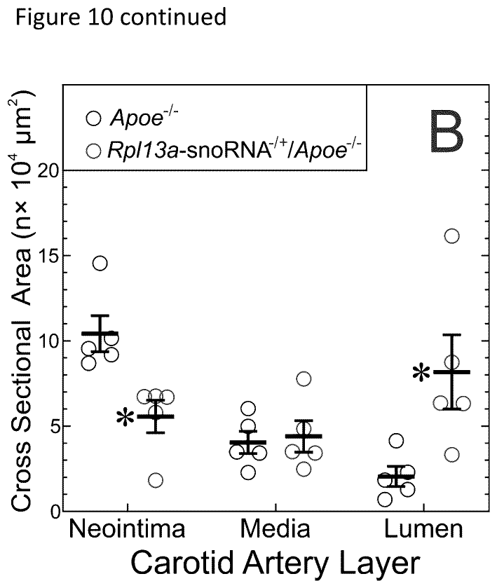

In yet another model, Apoe−/− mice with 50% reduced Rpl13a snoRNAs (snoRNA−/+, heterozygotes) developed 40% less carotid athero, foam cell formation, and SMC-derived foam cell formation19 after carotid endothelial denudation, a model for accelerated athero20 (FIG. 10 and not shown). Thus, Rpl13a snoRNAs not only aggravate athero but also promote SMC-to-foam cell transdifferentiation in 3 distinct athero models. Furthermore, it appears that even just a ˜50% reduction in snoRNA levels suffices to attenuate athero-auguring favorably for the success of ASO-based, snoRNA-targeted therapies.

In summary, we have now found that mice lacking the Rpl13a snoRNAs are significantly protected from athero in each of these disease models, without lowering LDL-C. In fact, Apoe−/−/snoKO mice have 20% higher LDL-C.

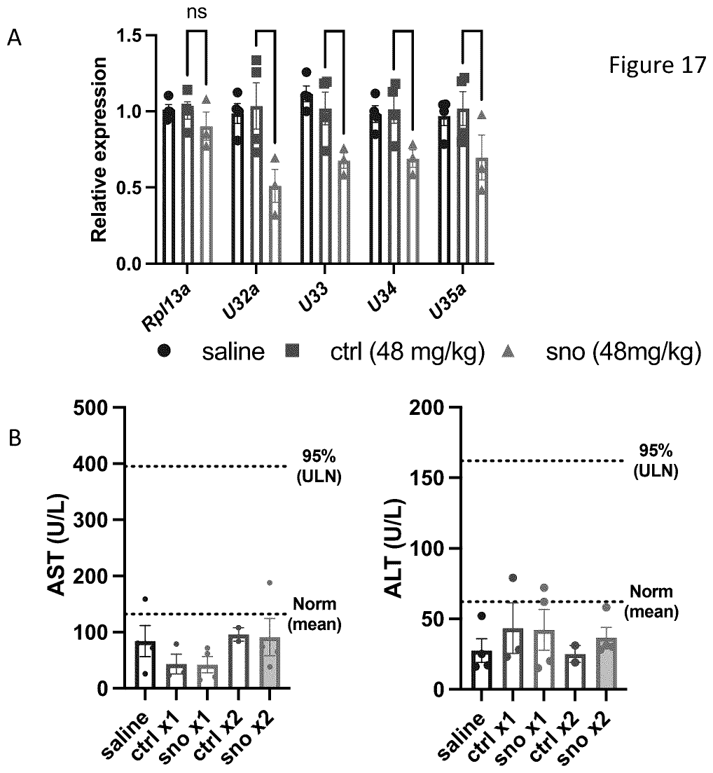

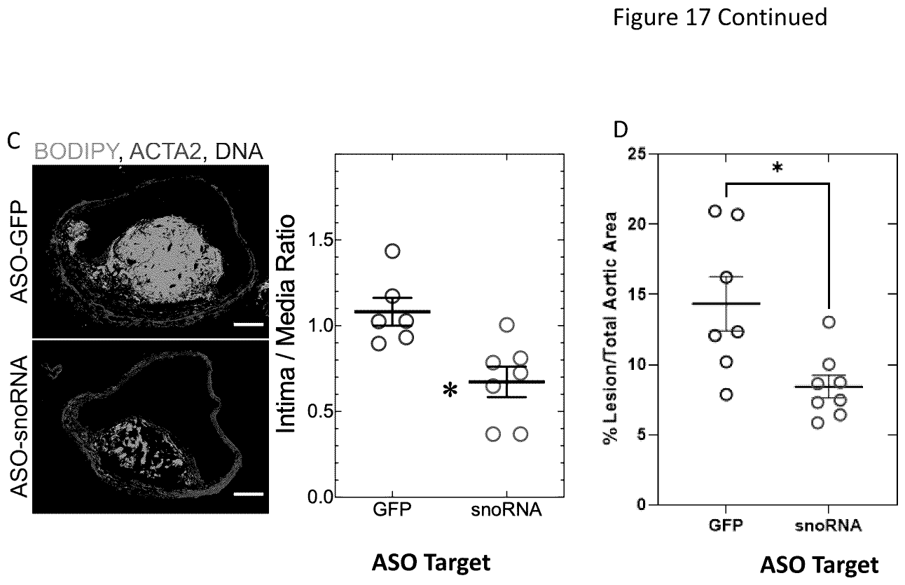

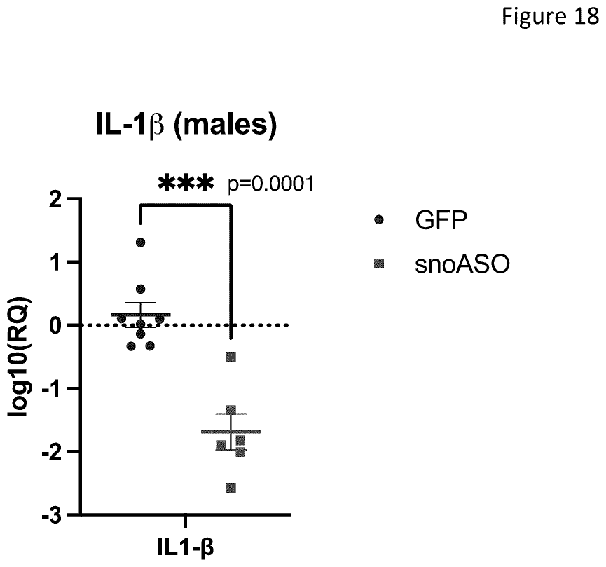

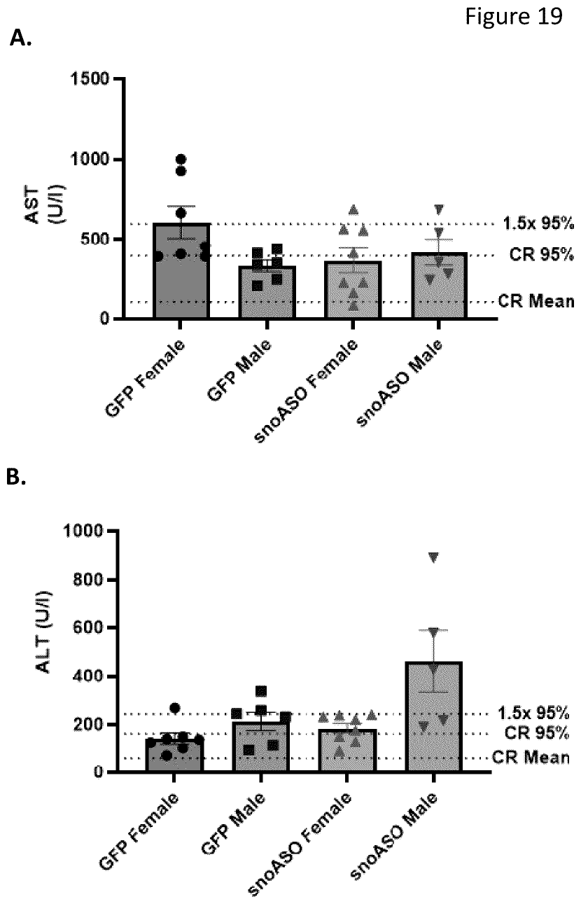

We have ongoing preliminary studies that address two aspects of our strategy, with short-term pilot funding for each. First, with Duke CTSI support (ends 6/31/2023), we are testing ASOs similar to those we have published (targeting the 3′-ASE, but now with 2′-methoxyethyl [2′-MOE] modification instead of LNA, and 5mdC to reduce immune reactivity) to see whether they protect mice from athero in the Apoe−/−/HFD model. The work is still in progress, but we have preliminary data confirming that ASOs can achieve significant knockdown of the Rpl13a snoRNAs in the aorta (30-50% with a single injection of 48 mg/kg) (FIG. 17A), with significant 30% reduction in athero, even though ASO administration was not initiated until after the onset of disease (FIG. 17C). We also saw a strong reduction in IL-1beta expression as a marker of athero-associated inflammation (FIG. 18). As expected, our preliminary testing shows that ASO administration is well-tolerated, without significant hepatotoxicity (FIG. 17B and FIG. 19).

### References for Example 2

1. Kassebaum N J, Arora M, Barber R M, Bhutta Z A, Brown J, Carter A, et al. Global, regional, and national disability-adjusted life-years (DALYs) for 315 diseases and injuries and healthy life expectancy (HALE), 1990-2015: a systematic analysis for the Global Burden of Disease Study 2015. The Lancet 2016; 388:1603-58.
2. Wang H, Naghavi M, Allen C, Barber R M, Bhutta Z A, Carter A, et al. Global, regional, and national life expectancy, all-cause mortality, and cause-specific mortality for 249 causes of death, 1980-2015: a systematic analysis for the Global Burden of Disease Study 2015. The Lancet 2016; 388:1459-544.
3. Vos T, Lim S S, Abbafati C, Abbas K M, Abbasi M, Abbasifard M, et al. Global burden of 369 diseases and injuries in 204 countries and territories, 1990-2019: a systematic analysis for the Global Burden of Disease Study 2019. The Lancet 2020; 396:1204-22.
4. Virani S S, Alonso A, Aparicio H J, Benjamin E J, Bittencourt M S, Callaway C W, et al. Heart Disease and Stroke Statistics-2021 Update. Circulation 2021; 143: e254-743.
5. Ritchey M D, Wall H K, Owens P L, Wright J S. Vital Signs: State-Level Variation in Nonfatal and Fatal Cardiovascular Events Targeted for Prevention by Million Hearts 2022. MMWR Morb Mortal Wkly Rep 2018; 67:974-82.
6. Vasan R S, Enserro D M, Xanthakis V, Beiser A S, Seshadri S. Temporal Trends in the Remaining Lifetime Risk of Cardiovascular Disease Among Middle-Aged Adults Across 6 Decades: The Framingham Study. Circulation 2022; 145:1324-38.
7. Liu C, Chen J, Chen H, Zhang T, He D, Luo Q, et al. PCSK9 Inhibition: From Current Advances to Evolving Future. Cells 2022; 11:2972.
8. Hansson G K, Hermansson A. The immune system in atherosclerosis. Nat Immunol 2011; 12:204-12.
9. Kattoor A J, Pothineni NVK, Palagiri D, Mehta J L. Oxidative Stress in Atherosclerosis. Curr Atheroscler Rep 2017; 19:42.
10. Li H, Horke S, Forstermann U. Vascular oxidative stress, nitric oxide and atherosclerosis. Atherosclerosis 2014; 237:208-19.
11. Bouabdallaoui N, Tardif J-C, Waters D D, Pinto F J, Maggioni A P, Diaz R, et al. Time-to-treatment initiation of colchicine and cardiovascular outcomes after myocardial infarction in the Colchicine Cardiovascular Outcomes Trial (COLCOT). European Heart Journal 2020; 41:4092-9.
12. Tardif J-C, Kouz S, Waters D D, Bertrand O F, Diaz R, Maggioni A P, et al. Efficacy and Safety of Low-Dose Colchicine after Myocardial Infarction. N Engl J Med 2019; 381:2497-505.
13. Ridker P M, Everett B M, Thuren T, MacFadyen J G, Chang W H, Ballantyne C, et al. Antiinflammatory Therapy with Canakinumab for Atherosclerotic Disease. N Engl J Med 2017; 377:1119-31.
14. Zhang L, Wu J-H, Elliott B A, Yin X, Snow K P, Freedman N J, et al. Abstract 15731: Rpl13a Small Nucleolar RNAs Promote Atherosclerosis and Oxidative Stress. Circulation 2022: A15731-A15731.
15. Michel C I, Holley C L, Scruggs B S, Sidhu R, Brookheart R T, Listenberger L L, et al. Small nucleolar RNAs U32a, U33 and U35a are critical mediators of metabolic stress. Cell Metab 2011; 14:33-44.
16. Lee J, Harris A N, Holley C L, Mahadevan J, Pyles K D, Lavagnino Z, et al. Rpl13a small nucleolar RNAs regulate systemic glucose metabolism. Journal of Clinical Investigation 2016; 126:4616-25.
17. Das S, Shah R, Dimmeler S, Freedman J E, Holley C, Lee J-M, et al. Noncoding RNAs in Cardiovascular Disease: Current Knowledge, Tools and Technologies for Investigation, and Future Directions: A Scientific Statement From the American Heart Association. Circ: Genomic and Precision Medicine 2020.
18. Elliott B A, Ho H-T, Ranganathan S V, Vangaveti S, Ilkayeva O, Abou Assi H, et al. Modification of messenger RNA by 2′-O-methylation regulates gene expression in vivo. Nat Commun 2019; 10:3401.
19. Wu J-H, Zhang L, Nepliouev I, Brian L, Huang T, Snow K P, et al. Drebrin attenuates atherosclerosis by limiting smooth muscle cell transdifferentiation. Cardiovascular Research 2022; 118:772-84.
20. Shah S H, Freedman N J, Zhang L, Crosslin D R, Stone D H, Haynes C, et al. Neuropeptide Y gene polymorphisms confer risk of early-onset atherosclerosis. PLOS Genet 2009; 5: e1000318.
21. Michel C I, Holley C L, Scruggs B S, Sidhu R, Brookheart R T, Listenberger L L, et al. Small Nucleolar RNAs U32a, U33, and U35a Are Critical Mediators of Metabolic Stress. Cell Metabolism 2011; 14:33-44.
22. Dhuri K, Bechtold C, Quijano E, Pham H, Gupta A, Vikram A, et al. Antisense Oligonucleotides: An Emerging Area in Drug Discovery and Development. JCM 2020; 9:2004.
23. Tran H, Moazami M P, Yang H, McKenna-Yasek D, Douthwright C L, Pinto C, et al. Suppression of mutant C9orf72 expression by a potent mixed backbone antisense oligonucleotide. Nat Med 2022; 28:117-24.
24. Benizri S, Gissot A, Martin A, Vialet B, Grinstaff M W, Barthelemy P. Bioconjugated Oligonucleotides: Recent Developments and Therapeutic Applications. Bioconjugate Chem 2019; 30:366-83.
25. Hvam M L, Cai Y, Dagnas-Hansen F, Nielsen J S, Wengel J, Kjems J, et al. Fatty Acid-Modified Gapmer Antisense Oligonucleotide and Serum Albumin Constructs for Pharmacokinetic Modulation. Molecular Therapy 2017; 25:1710-7.
26. Wolfrum C, Shi S, Jayaprakash K N, Jayaraman M, Wang G, Pandey R K, et al. Mechanisms and optimization of in vivo delivery of lipophilic siRNAs. Nat Biotechnol 2007; 25:1149-57.
27. Shatzkes K, Teferedegne B, Murata H. A simple, inexpensive method for preparing cell lysates suitable for downstream reverse transcription quantitative PCR. Sci Rep 2014; 4:4659.
28. Genoud V, Stortz M, Waisman A, Berardino B G, Verneri P, Dansey V, et al. Extraction-free protocol combining proteinase K and heat inactivation for detection of SARS-COV-2 by R T-qPCR. PLOS ONE 2021; 16: e0247792.
29. Shin M, Meda Krishnamurthy P, Devi G, Watts J K. Quantification of Antisense Oligonucleotides by Splint Ligation and Quantitative Polymerase Chain Reaction. Nucleic Acid Therapeutics 2022; 32:66-73.
30. Wooden J, Ciborowski P. Chromatin immunoprecipitation for human monocyte derived macrophages. Methods 2014; 70:89-96.
31. Kamola P J, Maratou K, Wilson P A, Rush K, Mullaney T, McKevitt T, et al. Strategies for In VivoScreening and Mitigation of Hepatotoxicity Associated with Antisense Drugs. Molecular Therapy—Nucleic Acids 2017; 8:383-94.
32. Kilanowska A, Studzińska S. In vivo and in vitro studies of antisense oligonucleotides—a review. RSC Adv 2020; 10:34501-16. https://doi.org/10.1039/DORA04978F.
33. Geary R S, Baker B F, Crooke S T. Clinical and Preclinical Pharmacokinetics and Pharmacodynamics of Mipomersen (Kynamro): A Second-Generation Antisense Oligonucleotide Inhibitor of Apolipoprotein B. Clinical Pharmacokinetics 2015; 54:133-46.
34. Wu J-H, Zhang L, Fanaroff A C, Cai X, Sharma K C, Brian L, et al. G Protein-Coupled Receptor Kinase-5 Attenuates Atherosclerosis by Regulating Receptor Tyrosine Kinases and 7-Transmembrane Receptors. ATVB 2012; 32:308-16.
35. Zhang L, Peppel K, Sivashanmugam P, Orman E S, Brian L, Exum S T, et al. Expression of tumor necrosis factor receptor-1 in arterial wall cells promotes atherosclerosis. Arterioscler Thromb Vasc Biol 2007; 27:1087-94.
36. Goettsch C, Hutcheson J D, Hagita S, Rogers M A, Creager M D, Pham T, et al. A single injection of gain-of-function mutant PCSK9 adeno-associated virus vector induces cardiovascular calcification in mice with no genetic modification. Atherosclerosis 2016; 251:109-18.
37. Roldan-Montero R, Perez-Saez J M, Cerro-Pardo I, Oller J, Martinez-Lopez D, Nunez E, et al. Galectin-1 prevents pathological vascular remodeling in atherosclerosis and abdominal aortic aneurysm. Sci Adv 2022; 8: eabm7322.
38. Katsuki S, K. Jha P, Lupieri A, Nakano T, Passos LSA, Rogers M A, et al. Proprotein Convertase Subtilisin/Kexin 9 (PCSK9) Promotes Macrophage Activation via LDL Receptor-Independent Mechanisms. Circ Res 2022; 131:873-89.
39. Jean-Charles P-Y, Wu J-H, Zhang L, Kaur S, Nepliouev I, Stiber J A, et al. USP20 (Ubiquitin-Specific Protease 20) Inhibits TNF (Tumor Necrosis Factor)-Triggered Smooth Muscle Cell Inflammation and Attenuates Atherosclerosis. ATVB 2018; 38:2295-305.
40. Zhang L, Wu J-H, Huang T-Q, Nepliouev I, Brian L, Zhang Z, et al. Drebrin regulates angiotensin Ilinduced aortic remodelling. Cardiovascular Research 2018; 114:1806-15.

### Example 3

### Methods

### Animals and Genotyping

Mice with targeted disruption of Rpl13a snoRNAs (snoKO) were used in this study. Animals were maintained on a standard chow diet and housed in a temperature-controlled environment with a 12-hour light/dark cycle. Genotyping of the mice was performed as previously described. All animal protocols were approved by the Duke University Institutional Animal Care and Use Committee.

### Vascular Injury Model

Endothelial injury was induced in the carotid arteries of WT and snoKO mice as previously described. Briefly, mice were anesthetized, and their carotid arteries were exposed. A flexible wire was inserted into the artery to induce endothelial injury. The animals were sacrificed at 4 and 6 weeks post-injury, and the carotid arteries were harvested for further analysis.

### Immunofluorescence Staining and Imaging

Carotid frozen sections were immunostained with appropriate primary antibodies and secondary antibodies conjugated to fluorescent dyes. Hoechst 33342 was used to counterstain DNA. Fluorescence photomicrographs were captured at original magnification ×400 using a fluorescence microscope. ImageJ software was used to quantify the immunofluorescence staining.

### ROS Measurement

Reactive oxygen species (ROS) levels in carotid tissue and isolated SMCs were measured using CellROX Orange Reagent and MitoSOX Red, following the manufacturer's instructions. Fluorescence intensity was quantified using a fluorescence microplate reader.

### SMC Proliferation and Migration Assays

WT and snoKO SMCs were isolated and cultured as previously described. Cell proliferation was assessed by counting cell numbers at 2, 4, 6, and 8 days after seeding. Cell migration was evaluated using a transwell migration assay in the presence or absence of PDGF stimulation.

### NFκB Activation

SMCs were stimulated with TNFα, and cell lysates were processed for immunoblot analysis. Phosphorylation of the NFκB p65 subunit on Ser536 was detected using a specific primary antibody, and β-actin was used as a loading control.

### Cox4i2 Expression

Cox4i2 transcript and protein levels were measured in mouse SMCs, aorta, and human HEK293T cells using quantitative real-time PCR and immunoblot analysis, respectively.

Collagen Type I detection

Carotid sections were immunostained for collagen type I, and fluorescence intensity was quantified using ImageJ software.

### Results

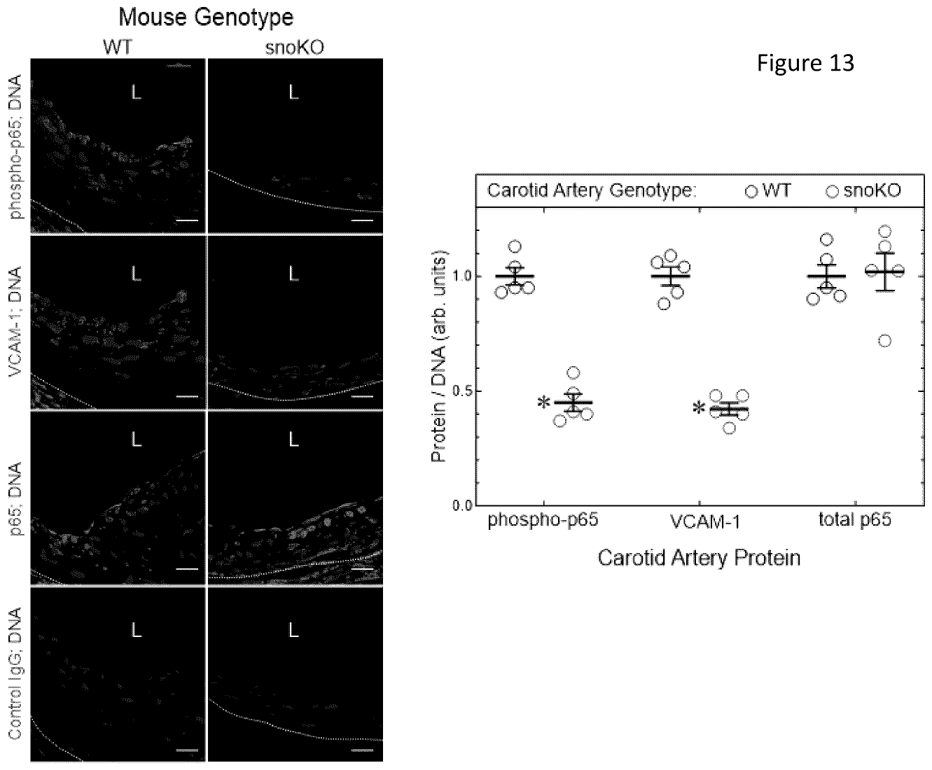

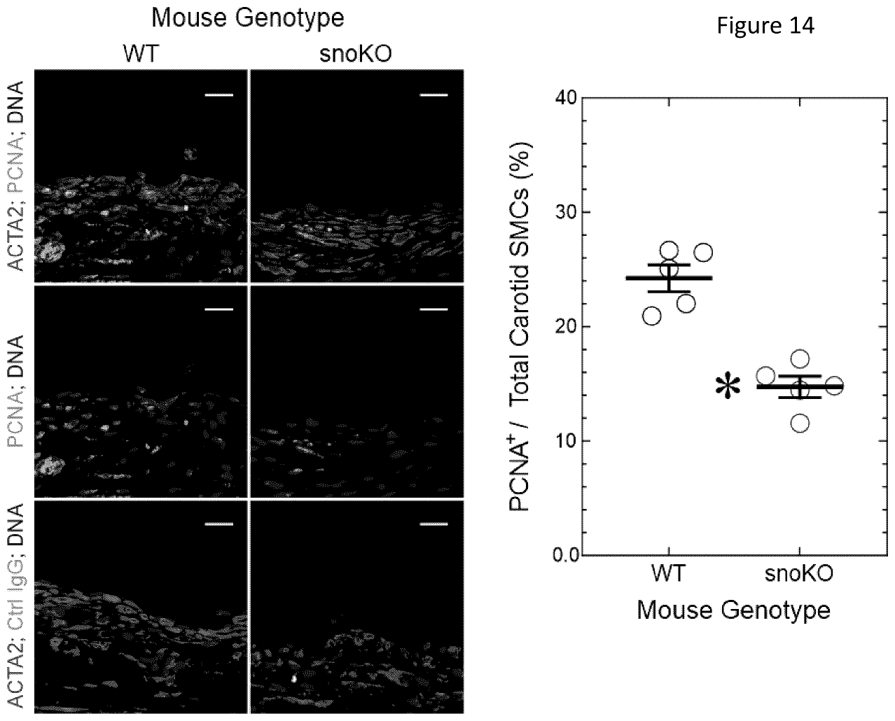

We showed that the loss of Rpl13a snoRNAs in snoKO mice resulted in lower levels of ROS and reduced neointimal hyperplasia compared to WT mice (FIG. 12). Moreover, our results demonstrated that Rpl13a snoRNA deficiency protected against the early stages of neointimal hyperplasia, as evidenced by decreased NFκB phosphorylation and VCAM-1 expression (FIG. 13). In support of these data, SMCs in snoKO carotids were less proliferative than those in WT mice (FIG. 14).

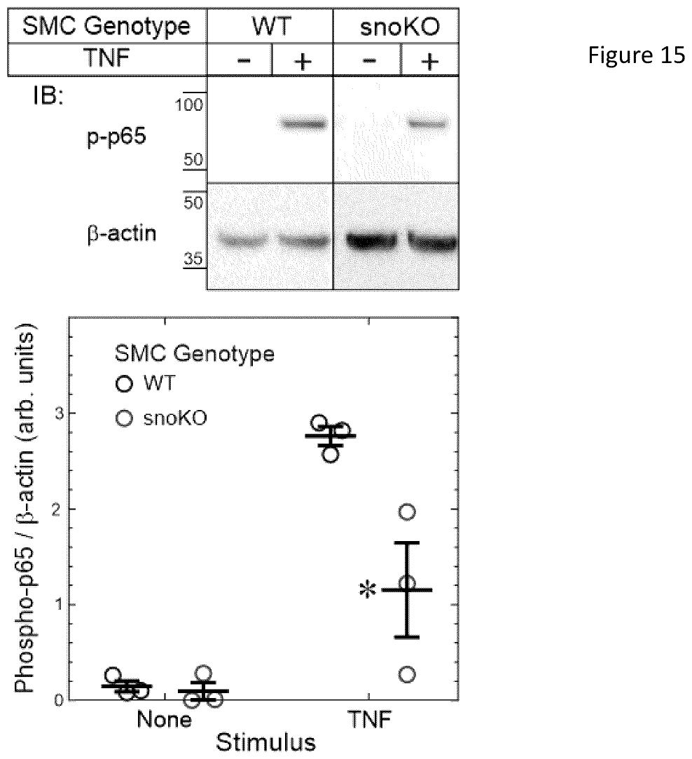

We observed that snoKO SMCs exhibited decreased ROS production, proliferation, and migration compared to WT SMCs (FIG. 15). A primary SMCs from snoKO mice were protected from NFκB activation upon atherogenic stimulation with TNFα. As Cox4i2 was significantly elevated in our proteomic analysis of WT and snoKO SMCs, we validated increased Cox412 protein expression in snoKO mouse SMCs, aorta, as well as in U32a and U51 knockout human HEK293T cells.

In summary, our results demonstrate that the loss of Rpl13a snoRNAs in mice leads to a reduction in ROS levels, neointimal hyperplasia, and SMC proliferation in a model of vascular injury. Additionally, the absence of Rpl13a snoRNAs offers protection against the early stages of neointimal hyperplasia, as evidenced by decreased NFκB phosphorylation and VCAM-1 expression. The data also reveal that snoKO SMCs have diminished ROS production, proliferation, and migration capabilities compared to WT SMCs.

Furthermore, we found that primary SMCs isolated from snoKO mice are protected from NFκB activation in response to atherogenic stimulation. The increase in Cox4i2 protein expression observed in snoKO mouse SMCs, aorta, and human U32a and U51 KO HEK293T cells suggests a potential role for Rpl13a snoRNAs in modulating Cox4i2 expression and could explain the observed increase detected in our proteomic analyses (Table 1). This specific observation in the U32a/U51 KO cells suggest that the transcript is directly regulated by the 5′ D′-box ASE region of U32a.

Lastly, our data confirm that baseline ROS levels are lower in the aorta of snoKO mice, further supporting the protective effects of Rpl13a snoRNA deficiency in the context of vascular injury. Overall, these findings provide valuable insights into the role of Rpl13a snoRNAs in vascular remodeling and highlight their potential as therapeutic targets for the treatment of vascular diseases.

### References for Example 3 (see Example 1)

### Example 4

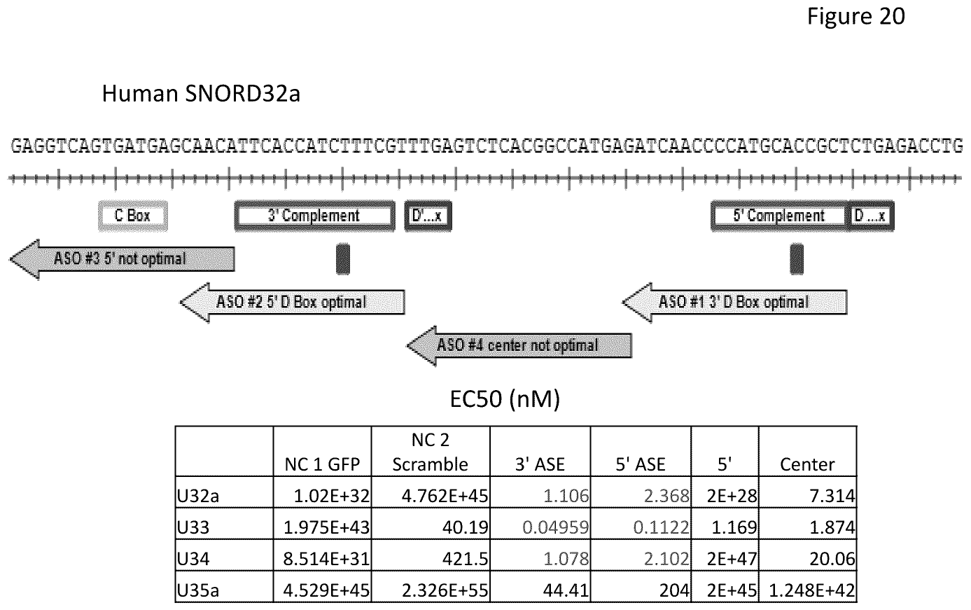

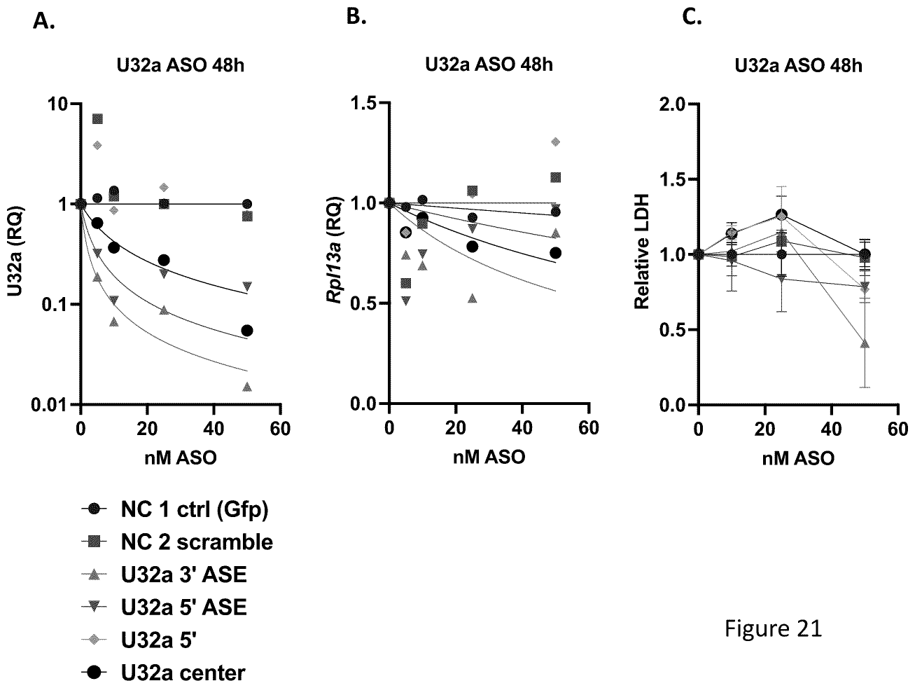

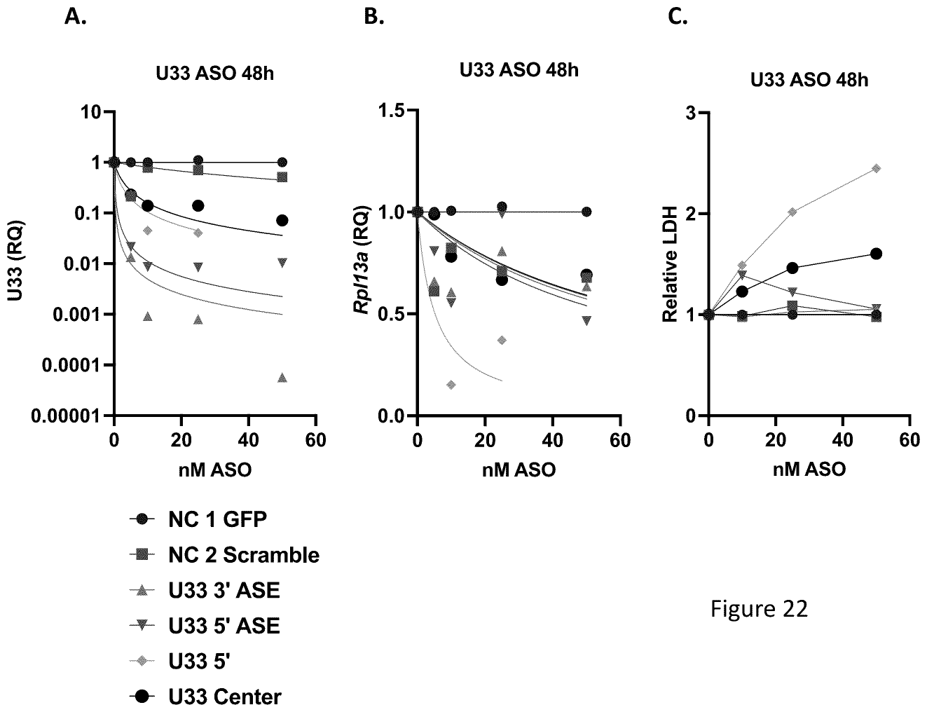

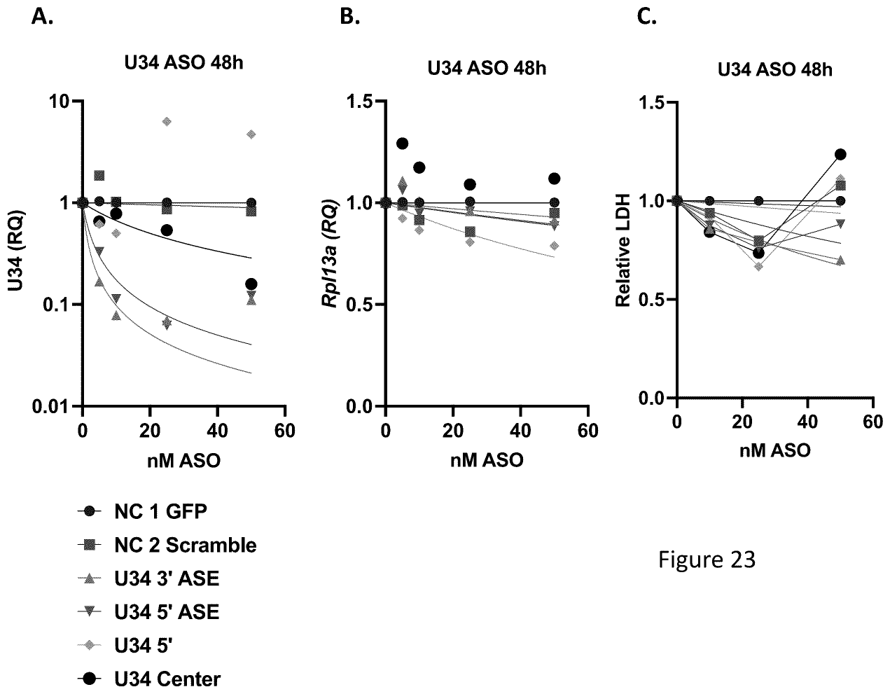

1. ASOs targeting either the 5′- or 3′-antisense elements (ASE) of snoRNAs U32A, U33, and U34 are highly effective for target knockdown (low nM inhibitors when delivered using lipofection, with no apparent toxicity (GO). ASOs targeting the center or 5′-end of these snoRNAs were ineffective and some showed toxicity (NO GO). We have chosen to advance ASOs targeting both the 5′ and 3′-ASE for these snoRNAs to additional testing in subsequent Tasks. Supporting data are shown in FIGS. 20-23.
2. Testing of ASOs targeting snoRNA U35A have been limited by the sensitivity of our newly-developed multiplex assay. Limiting factors include a highly related snoRNA U35B and inefficient RT-qPCR amplification that is problematic in the high-throughput multiplex format. We are continuing to troubleshoot this and may need to test U35A ASOs without the high-throughput benefit of multiplexed RT-qPCR. Based on our prior experience and the results above, we expect that targeting U35A at the 5′ and/or 3′-ASE will be the most effective approach.
3. A sample of our data for ASOs targeting U32A in HeLa cells is shown below. Panel A: U32A knockdown, dose-response curves. EC50 for 5′ and 3′-ASE are ˜1-2 nM, compared to negative controls (NC). Panel B: No ASO toxicity when targeting the 5′ or 3′-ASE, as measured by LDH release.

One skilled in the art will readily appreciate that the present disclosure is well adapted to carry out the objects and obtain the ends and advantages mentioned, as well as those inherent therein. The present disclosure described herein are presently representative of preferred embodiments, are exemplary, and are not intended as limitations on the scope of the present disclosure. Changes therein and other uses will occur to those skilled in the art which are encompassed within the spirit of the present disclosure as defined by the scope of the claims.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

1. A composition comprising an antisense oligonucleotide capable of binding an Rpl13a snoRNA, wherein the antisense oligonucleotide comprises at least one of SEQ ID NO: 17-32, 77-84, a sequence with at least 90% identity to SEQ ID NO: 17-32, 77-84 and combinations thereof.

2. The composition of claim 1, wherein the antisense oligonucleotide is modified to comprise a phosphorothioate backbone, 5-methylcytosine and the first five 5′ and last five 3′ nucleotides comprise 2′-O-methoxy-ethyl bases (2′-MOE) bases.

3. The composition of claim 2, wherein the antisense oligonucleotide comprises at least one of SEQ ID NO: 33-72 or a sequence with at least 95% identity to SEQ ID NO: 33-72 and combinations thereof.

4. The composition of claim 1, additionally comprising at least one of SEQ ID NO: 1-4, 73-76 or sequence with at least 95% identity to SEQ ID NO: 1-4, 73-76 and combinations thereof.

5. The composition of claim 1, wherein the Rpl13a snoRNA comprises at least one of U32a, U33, U34 or U35a.

6. The composition of claim 1, wherein the composition comprises a set of antisense oligonucleotides with at least one antisense oligonucleotide in the set capable of binding to each of U32a, U33, U34 or U35a.

7. A pharmaceutical composition comprising the composition of claim 1 and a pharmaceutical acceptable excipient, carrier or diluent.

8. A method of treating and/or preventing cardiovascular disease in a subject in need thereof, the method comprising administering a therapeutically effective amount of an inhibitor of a Rpl13a snoRNA.

9. The method of claim 8, wherein the inhibitor is capable of reducing and/or inhibiting a Rpl13a snoRNA in the subject as compared to a control.

10. (canceled)

11. The method of claim 8, wherein the cardiovascular disease is atherosclerosis.

12. The method of claim 8, wherein the Rpl13a snoRNA comprises U32a, U33, U34, or U35a.

13. A method of treating and/or preventing cardiovascular disease in a subject in need thereof, the method comprising administering a therapeutically effective amount of the composition of claim 1.

14. The method of claim 8, wherein the inhibitor is selected from the group consisting of SEQ ID NO: 17-84 or a sequence with at least 95% identity to SEQ ID NO: 17-84 and combinations thereof.

15. The method of claim 8, wherein the inhibitor is selected from the group consisting of SEQ ID NO: 1-4 or a sequence with at least 95% identity to SEQ ID NO: 1-4 and combinations thereof.

16. A method of preventing and/or treating inflammation in a subject, the method comprising administering a therapeutically effective amount of an inhibitor of a Rpl13a snoRNA.

17. The method of claim 16, wherein the Rpl13a snoRNA comprises U32a, U33, U34 or U35 or combinations thereof.

18. A method of preventing and/or treating inflammation in a subject, the method comprising administering a therapeutically effective amount of an inhibitor of a Rpl13a snoRNA, wherein the inhibitor comprises the composition of claim 1.

19. The method of claim 16, wherein the inhibitor is selected from the group consisting of SEQ ID NO: 17-84 or a sequence with at least 95% identity to SEQ ID NO: 17-84 or combinations thereof.

20. The method of claim 16, wherein the inhibitor is selected from the group consisting of SEQ ID NO: 1-4 or a sequence with at least 95% identity to SEQ ID NO: 1-4 or combinations thereof.

21. (canceled)

22. The method of claim 16, wherein treating the inflammation results in decreased IL-1B, ROS and/or VCAM1.

23.-26. (canceled)


SEQUENCE LISTING (84 sequences)

SEQ ID NO: 1 - DNA, 20 bp, synthetic construct
  source 1..20: mol_type = other DNA
  modified_base 5: mod_base = OTHER; note = Uracil
  modified_base 17: mod_base = OTHER; note = Uracil
  modified_base 19: mod_base = OTHER; note = Uracil
  modified_base 1..5: mod_base = OTHER; mod_base = OTHER; note = 2-O-methoxyribonucleotide
  modified_base 16..20: mod_base = OTHER; note = 2-O-methoxyribonucleotide
  modified_base 1..20: mod_base = OTHER; note = phosphorothioate backbone
  misc_feature 6..15: note = DNA
gcggtgcatgggttgatctc

SEQ ID NO: 2 - DNA, 20 bp, synthetic construct
  source 1..20: mol_type = other DNA
  modified_base 1: mod_base = OTHER; note = Uracil
  modified_base 4: mod_base = OTHER; note = Uracil
  modified_base 18: mod_base = OTHER; note = Uracil
  modified_base 1..5: mod_base = OTHER; note = 2-O-methoxyribonucleotide
  modified_base 16..20: mod_base = OTHER; note = 2-O-methoxyribonucleotide
  modified_base 1..20: mod_base = OTHER; note = phosphorothioate backbone
  misc_feature 6..15: note = DNA
tggtagtgcatgtagagtca

SEQ ID NO: 3 - DNA, 20 bp, synthetic construct
  source 1..20: mol_type = other DNA
  modified_base 1..5: mod_base = OTHER; note = 2-O-methoxyribonucleotide
  modified_base 16..20: mod_base = OTHER; note = 2-O-methoxyribonucleotide
  modified_base 1..20: mod_base = OTHER; note = phosphorothioate backbone
  misc_feature 6..15: note = DNA
  modified_base 4: mod_base = OTHER; note = uracil
cagtgctgctttcatgagga

SEQ ID NO: 4 - DNA, 20 bp, synthetic construct
  source 1..20: mol_type = other DNA
  modified_base 1..2: mod_base = OTHER; note = Uracil
  modified_base 17: mod_base = OTHER; note = Uracil
  modified_base 1..5: mod_base = OTHER; note = 2-O-methoxyribonucleotide
  modified_base 16..20: mod_base = OTHER; note = 2-O-methoxyribonucleotide
  modified_base 1..20: mod_base = OTHER; note = phosphorothioate backbone
  misc_feature 6..15: note = DNA
ttagcctttggcattatcgg

SEQ ID NO: 5 - DNA, 84 bp, Homo sapiens
  source 1..84: mol_type = genomic DNA
gaggtcagtgatgagcaacattcaccatctttcgtttgagtctcacggccatgagatcaaccccatgcaccgctctgagacctg

SEQ ID NO: 6 - DNA, 85 bp, Homo sapiens
  source 1..85: mol_type = genomic DNA
gcggccggtgatgagaacttctcccactcacattcgagtttcccgaccatgagatgactccacatgcactaccatctgaggccac

SEQ ID NO: 7 - DNA, 71 bp, Homo sapiens
  source 1..71: mol_type = genomic DNA
tggcgtccatgatgttccgcaactacctacattgtttgatcctcatgaaagcagcactggctgagacgcca

SEQ ID NO: 8 - DNA, 86 bp, Homo sapiens
  source 1..86: mol_type = genomic DNA
ggcagatgatgtccttatctcacgatggtctgcggatgtccctgtgggaatggcgacaatgccaatggcttagctgatgccaggag

SEQ ID NO: 9 - DNA, 70 bp, Homo sapiens
  source 1..70: mol_type = genomic DNA
gttgcatgatgaataaaatcaaatcaccatctttcggctgagttcgtgatggatttgcttttttctgatt

SEQ ID NO: 10 - DNA, 88 bp, Homo sapiens
  source 1..88: mol_type = genomic DNA
tggcagatgatgtttgttttcacgatggtcttcagatgcccacgtgggcactgctgagaaagccacttggtaaaactgatgccggaaa

SEQ ID NO: 11 - DNA, 1117 bp, Homo sapiens
  source 1..1117: mol_type = genomic DNA
cttttccaagcggctgccgaagatggcggaggtgcaggtcctggtgcttgatggtcgaggccatctcctgggccgcctggcggccatcgtggctaaacaggccggaaggtggtggtcgtacgctgtgaaggcatcaacatttctggcaatttctacagaaacaagttgaagtacctggctttcctccgcaagcggatgaacaccaacccttcccgaggcccctaccacttccgggcccccagccgcatcttctggcggaccgtgcgaggtatgctgccccacaaaaccaagcgaggccaggccgctctggaccgtctcaaggtgtttgacggcatcccaccgccctacgacaagaaaaagcggatggtggttcctgctgccctcaaggtcgtgcgtctgaagcctacaagaaagtttgcctatctggggcgcctggctcacgaggttggctggaagtaccaggcagtgacagccaccctggaggagaagaggaaagagaaagccaagatccactaccggaagaagaaacagctcatgaggctacggaaacaggccgagaagaacgtggagaagaaaattgacaaatacacagaggtcctcaagacccacggactcctggtctgagcccaataaagactgttaattcctcatgcgttgcctgcccttcctccattgttgccctggaatgtacgggacccaggggcagcagcagtccaggtgccacaggcagccctgggacataggaagctgggagcaaggaaagggtcttagtcactgcctcccgaagttgcttgaaagcactcggagaattgtgcaggtgtcatttatctatgaccaataggaagagcaaccagttactatgagtgaaagggagccagaagactgattggagggccctatcttgtgagtggggcatctgttggactttccacctggtcatatactctgcagctgttagaatgtgcaagcacttggggacagcatgagcttgctgttgtacacagggtatttctagaagcagaaatagactgggaagatgcacaaccaaggggttacaggcatcgcccatgctcctcacctgtattttgtaatcagaaataaattgcttttaaagaaa

SEQ ID NO: 12 - DNA, 1127 bp, Homo sapiens
  source 1..1127: mol_type = genomic DNA
cttttccaagcggctgccgaagatggcggaggtgcaggtcctggtgcttgatggtcgaggccatctcctgggccgcctggcggccatcgtggctaaacaggtactgctgggccggaaggtggtggtcgtacgctgtgaaggcatcaacatttctggcaatttctacagaaacaagttgaagtacctggctttcctccgcaagcggatgaacaccaacccttcccgaggcccctaccacttccgggcccccagccgcatcttctggcggaccgtgcgaggtatgctgccccacaaaaccaagcgaggccaggccgctctggaccgtctcaaggtgtttgacggcatcccaccgccctacgacaagaaaaagcggatggtggttcctgctgccctcaaggtcgtgcgtctgaagcctacaagaaagtttgcctatctggggcgcctggctcacgaggttggctggaagtaccaggcagtgacagccaccctggaggagaagaggaaagagaaagccaagatccactaccggaagaagaaacagctcatgaggctacggaaacaggccgagaagaacgtggagaagaaaattgacaaatacacagaggtcctcaagacccacggactcctggtctgagcccaataaagactgttaattcctcatgcgttgcctgcccttcctccattgttgccctggaatgtacgggacccaggggcagcagcagtccaggtgccacaggcagccctgggacataggaagctgggagcaaggaaagggtcttagtcactgcctcccgaagttgcttgaaagcactcggagaattgtgcaggtgtcatttatctatgaccaataggaagagcaaccagttactatgagtgaaagggagccagaagactgattggagggccctatcttgtgagtggggcatctgttggactttccacctggtcatatactctgcagctgttagaatgtgcaagcacttggggacagcatgagcttgctgttgtacacagggtatttctagaagcagaaatagactgggaagatgcacaaccaaggggttacaggcatcgcccatgctcctcacctgtattttgtaatcagaaataaattgcttttaaagaaa

SEQ ID NO: 13 - protein, 203 aa, Homo sapiens [sequence omitted]

SEQ ID NO: 14 - protein, 203 aa, Mus musculus
MAEGQVLVLDGRGHLLGRLAAIVAKQVLLGRKVVVVRCEGINISGNFYRNKLKYLAFLRKRMNTNPSRGPYHFRAPSRIFWRTVRGMLPHKTKRGQAALERLKVLDGIPPPYDKKKRMVVPAALKVVRLKPTRKFAYLGRLAHEVGWKYQAVTATLEEKRKEKAKMHYRKKKQILRLRKQAEKNVEKKICKFTEVLKTNGLLV

SEQ ID NO: 15 - DNA, 1051 bp, Homo sapiens
  source 1..1051: mol_type = genomic DNA
cttttccaagcggctgccgaagatggcggaggtgcaggtcctggtgcttgatggtcgaggccatctcctgggccgcctggcggccatcgtggctaaacagtgaagtacctggctttcctccgcaagcggatgaacaccaacccttcccgaggcccctaccacttccgggcccccagccgcatcttctggcggaccgtgcgaggtatgctgccccacaaaaccaagcgaggccaggccgctctggaccgtctcaaggtgtttgacggcatcccaccgccctacgacaagaaaaagcggatggtggttcctgctgccctcaaggtcgtgcgtctgaagcctacaagaaagtttgcctatctggggcgcctggctcacgaggttggctggaagtaccaggcagtgacagccaccctggaggagaagaggaaagagaaagccaagatccactaccggaagaagaaacagctcatgaggctacggaaacaggccgagaagaacgtggagaagaaaattgacaaatacacagaggtcctcaagacccacggactcctggtctgagcccaataaagactgttaattcctcatgcgttgcctgcccttcctccattgttgccctggaatgtacgggacccaggggcagcagcagtccaggtgccacaggcagccctgggacataggaagctgggagcaaggaaagggtcttagtcactgcctcccgaagttgcttgaaagcactcggagaattgtgcaggtgtcatttatctatgaccaataggaagagcaaccagttactatgagtgaaagggagccagaagactgattggagggccctatcttgtgagtggggcatctgttggactttccacctggtcatatactctgcagctgttagaatgtgcaagcacttggggacagcatgagcttgctgttgtacacagggtatttctagaagcagaaatagactgggaagatgcacaaccaaggggttacaggcatcgcccatgctcctcacctgtattttgtaatcagaaataaattgcttttaaagaaa

SEQ ID NO: 16 - DNA, 1039 bp, Mus musculus
  source 1..1039: mol_type = genomic DNA
gacctcctcctttcccaggcggctgccgaagatggcggaggggcaggttctggtattggatggccgaggccatcttcttggccgcctggcggccattgtggccaagcaggtacttctgggccggaaggtggtggtcgtacgctgtgaaggcatcaacatttctggaaacttctacagaaacaagttaaagtatctggcctttctccggaagcggatgaataccaacccctcccgaggcccctaccacttccgagcccccagccgcattttctggcgcactgtgcgaggcatgctgccccacaagaccaagagaggccaggctgccctggagcgcctcaaggtgttggatgggatccctccaccctatgacaagaaaaagcggatggtggtccctgctgctctcaaggttgttcggctgaagcctaccagaaagtttgcttacctggggcgtctggcgcatgaggtcgggtggaagtaccaggcagtgacagccactctggaggagaaacggaaggaaaaggccaagatgcactatcggaagaagaagcagatcttgaggttacggaaacaggcagaaaagaatgtggagaagaaaatctgcaagttcacagaggtcctcaagaccaacggactcctggtgtgaacccaataaagactgtttgcctcatgcctgcctggcctgcccttcctccgccgccaactagggaagtggggaccaaaggttccttaggcactgctcctgtgggtagaggggacattagagagctgacagcgcaccacctgcatgagtttttattaaagtgcaaaccatgggatgaatcagttgagcttcagtgttgaaaatgagtagcagggctgccccacccacctgaccaagtaccctattctgcagctatgaaaatgagatctgcacatgagctggggttcacaagtgcacacttggagcactgccttgctccttcccagcagaccacaaagcagtatttttctggaggattttatgtgctaataaattatttgacttaaaaaaaaaaaaaaa

SEQ ID NO: 17 - DNA, 20 bp, synthetic construct
  source 1..20: mol_type = other DNA
agcggtgcatggggttgatc

SEQ ID NO: 18 - DNA, 20 bp, synthetic construct
  source 1..20: mol_type = other DNA
acgaaagatggtgaatgttg

SEQ ID NO: 19 - DNA, 20 bp, synthetic construct
  source 1..20: mol_type = other DNA
tgttgctcatcactgacctc

SEQ ID NO: 20 - DNA, 20 bp, synthetic construct
  source 1..20: mol_type = other DNA
ctcatggccgtgagactcaa

SEQ ID NO: 21 - DNA, 20 bp, synthetic construct
  source 1..20: mol_type = other DNA
atggtagtgcatgtggagtc

SEQ ID NO: 22 - DNA, 20 bp, synthetic construct
  source 1..20: mol_type = other DNA
atgtgagtgggagaagttct

SEQ ID NO: 23 - DNA, 20 bp, synthetic construct
  source 1..20: mol_type = other DNA
aagttctcatcaccggccgc

SEQ ID NO: 24 - DNA, 20 bp, synthetic construct
  source 1..20: mol_type = other DNA
ctcatggtcgggaaactcga

SEQ ID NO: 25 - DNA, 20 bp, synthetic construct
  source 1..20: mol_type = other DNA
ccagtgctgctttcatgagg

SEQ ID NO: 26 - DNA, 20 bp, synthetic construct
  source 1..20: mol_type = other DNA
acaatgtaggtagttgcgga

SEQ ID NO: 27 - DNA, 20 bp, synthetic construct
  source 1..20: mol_type = other DNA
tgaggatcaaacaatgtagg

SEQ ID NO: 28 - DNA, 20 bp, synthetic construct
  source 1..20: mol_type = other DNA
tcatgaggatcaaacaatgt

SEQ ID NO: 29 - DNA, 20 bp, synthetic construct
  source 1..20: mol_type = other DNA
ctaagccattggcattgtcg

SEQ ID NO: 30 - DNA, 20 bp, synthetic construct
  source 1..20: mol_type = other DNA
cagaccatcgtgagataagg

SEQ ID NO: 31 - DNA, 20 bp, synthetic construct
  source 1..20: mol_type = other DNA
agataaggacatcatctgcc

SEQ ID NO: 32 - DNA, 20 bp, synthetic construct
  source 1..20: mol_type = other DNA
cattcccacagggacatccg

SEQ ID NO: 33 - DNA, 20 bp, synthetic construct
  source 1..20: mol_type = other DNA
  modified_base 1..5: mod_base = OTHER; note = 2'-MOE RNA
  modified_base 16..20: mod_base = OTHER; note = 2'-MOE RNA
  modified_base 1..20: mod_base = OTHER; note = Phosphorothioate backbone
  modified_base 1..20: mod_base = m5c
  misc_feature 6..15: note = DNA
agcggtgcatggggttgatc

SEQ ID NO: 34 - DNA, 20 bp, synthetic construct
  source 1..20: mol_type = other DNA
  modified_base 1..5: mod_base = OTHER; note = 2'-MOE RNA
  modified_base 16..20: mod_base = OTHER; note = 2'-MOE RNA
  modified_base 1..20: mod_base = m5c
  modified_base 1..20: mod_base = OTHER; note = Phosphorothioate backbone
  misc_feature 6..15: note = DNA
acgaaagatggtgaatgttg

SEQ ID NO: 35 - DNA, 20 bp, synthetic construct
  source 1..20: mol_type = other DNA
  modified_base 1..5: mod_base = OTHER; note = 2'-MOE RNA
  modified_base 16..20: mod_base = OTHER; note = 2'-MOE RNA
  modified_base 1..20: mod_base = m5c
  modified_base 1..20: mod_base = OTHER; note = Phosphorothioate backbone
  misc_feature 6..15: note = DNA
tgttgctcatcactgacctc

SEQ ID NO: 36 - DNA, 20 bp, synthetic construct
  source 1..20: mol_type = other DNA
  modified_base 1..5: mod_base = OTHER; note = 2'-MOE RNA
  modified_base 16..20: mod_base = OTHER; note = 2'-MOE RNA
  modified_base 1..20: mod_base = OTHER; note = Phosphorothioate backbone
  modified_base 1..20: mod_base = m5c
  misc_feature 6..15: note = DNA
ctcatggccgtgagactcaa

SEQ ID NO: 37 - DNA, 20 bp, synthetic construct
  source 1..20: mol_type = other DNA
  modified_base 1..5: mod_base = OTHER; note = 2'MOE RNA
  modified_base 16..20: mod_base = OTHER; note = 2'-MOE RNA
  modified_base 1..20: mod_base = OTHER; note = Phosphorothioate backbone
  modified_base 1..20: mod_base = m5c
  misc_feature 6..15: note = DNA
atggtagtgcatgtggagtc

SEQ ID NO: 38 - DNA, 20 bp, synthetic construct
  source 1..20: mol_type = other DNA
  modified_base 1..5: mod_base = OTHER; note = 2'-MOE RNA
  modified_base 16..20: mod_base = OTHER; note = 2'-MOE RNA
  modified_base 1..20: mod_base = OTHER; note = Phosphorothioate backbone
  modified_base 1..20: mod_base = m5c
  misc_feature 6..15: note = DNA
atgtgagtgggagaagttct

SEQ ID NO: 39 - DNA, 20 bp, synthetic construct
  source 1..20: mol_type = other DNA
  modified_base 1..5: mod_base = OTHER; note = 2'-MOE
  modified_base 16..20: mod_base = OTHER; note = 2'-MOE
  modified_base 1..20: mod_base = OTHER; note = Phosphorothioate backbone
  modified_base 1..20: mod_base = m5c
  misc_feature 6..15: note = DNA
aagttctcatcaccggccgc

SEQ ID NO: 40 - DNA, 20 bp, synthetic construct
  source 1..20: mol_type = other DNA
  modified_base 1..5: mod_base = OTHER; note = 2'-MOE
  modified_base 16..20: mod_base = OTHER; note = 2'-MOE
  modified_base 1..20: mod_base = OTHER; note = Phosphorothioate backbone
  modified_base 1..20: mod_base = m5c
  misc_feature 6..15: note = DNA
ctcatggtcgggaaactcga

SEQ ID NO: 41 - DNA, 20 bp, synthetic construct
  source 1..20: mol_type = other DNA
  modified_base 1..5: mod_base = OTHER; note = 2'-MOE
  modified_base 16..20: mod_base = OTHER; note = 2'-MOE
  modified_base 1..20: mod_base = OTHER; note = Phosphorothioate backbone
  modified_base 1..20: mod_base = m5c
  misc_feature 6..15: note = DNA
ccagtgctgctttcatgagg

SEQ ID NO: 42 - DNA, 20 bp, synthetic construct
  source 1..20: mol_type = other DNA
  modified_base 1..5: mod_base = OTHER; note = 2'-MOE
  modified_base 16..20: mod_base = OTHER; note = 2'-MOE
  modified_base 1..20: mod_base = OTHER; note = Phosphorothioate backbone
  modified_base 1..20: mod_base = m5c
  misc_feature 6..15: note = DNA
acaatgtaggtagttgcgga

SEQ ID NO: 43 - DNA, 20 bp, synthetic construct
  source 1..20: mol_type = other DNA
  modified_base 1..5: mod_base = OTHER; note = 2'-MOE
  modified_base 16..20: mod_base = OTHER; note = 2'-MOE
  modified_base 1..20: mod_base = OTHER; note = Phosphorothioate backbone
  modified_base 1..20: mod_base = m5c
  misc_feature 6..15: note = DNA
tgaggatcaaacaatgtagg

SEQ ID NO: 44 - DNA, 20 bp, synthetic construct
  source 1..20: mol_type = other DNA
  modified_base 1..5: mod_base = OTHER; note = 2'-MOE
  modified_base 16..20: mod_base = OTHER; note = 2'-MOE
  modified_base 1..20: mod_base = OTHER; note = Phosphorothioate backbone
  modified_base 1..20: mod_base = m5c
  misc_feature 6..15: note = DNA
tcatgaggatcaaacaatgt

SEQ ID NO: 45 - DNA, 20 bp, synthetic construct
  source 1..20: mol_type = other DNA
  modified_base 1..5: mod_base = OTHER; note = 2'-MOE
  modified_base 16..20: mod_base = OTHER; note = 2'-MOE
  modified_base 1..20: mod_base = OTHER; note = Phosphorothioate backbone
  modified_base 1..20: mod_base = m5c
  misc_feature 6..15: note = DNA
ctaagccattggcattgtcg

SEQ ID NO: 46 - DNA, 20 bp, synthetic construct
  source 1..20: mol_type = other DNA
  modified_base 1..5: mod_base = OTHER; note = 2'-MOE
  modified_base 16..20: mod_base = OTHER; note = 2'-MOE
  modified_base 1..20: mod_base = OTHER; note = Phosphorothioate backbone
  modified_base 1..20: mod_base = m5c
  misc_feature 6..15: note = DNA
cagaccatcgtgagataagg

SEQ ID NO: 47 - DNA, 20 bp, synthetic construct
  source 1..20: mol_type = other DNA
  modified_base 1..5: mod_base = OTHER; note = 2'-MOE
  modified_base 16..20: mod_base = OTHER; note = 2'-MOE
  modified_base 1..20: mod_base = OTHER; note = Phosphorothioate backbone
  modified_base 1..20: mod_base = m5c
  misc_feature 6..15: note = DNA
agataaggacatcatctgcc

SEQ ID NO: 48 - DNA, 20 bp, synthetic construct
  source 1..20: mol_type = other DNA
  modified_base 1..5: mod_base = OTHER; note = 2'-MOE
  modified_base 16..20: mod_base = OTHER; note = 2'-MOE
  modified_base 1..20: mod_base = OTHER; note = Phosphorothioate backbone
  modified_base 1..20: mod_base = m5c
  misc_feature 6..15: note = DNA
cattcccacagggacatccg

SEQ ID NO: 49 - DNA, 18 bp, synthetic construct
  source 1..18: mol_type = other DNA
  modified_base 1..5: mod_base = OTHER; note = 2'-MOE
  modified_base 14..18: mod_base = OTHER; note = 2'-MOE
  modified_base 1..18: mod_base = OTHER; note = Phosphorothioate backbone
  modified_base 1..18: mod_base = m5c
  misc_feature 6..13: note = DNA
gcggtgcatggggttgat

SEQ ID NO: 50 - DNA, 18 bp, synthetic construct
  source 1..18: mol_type = other DNA
  modified_base 1..5: mod_base = OTHER; note = 2'-MOE
  modified_base 14..18: mod_base = OTHER; note = 2'-MOE
  modified_base 1..18: mod_base = OTHER; note = Phosphorothioate backbone
  modified_base 1..18: mod_base = m5c
  misc_feature 6..13: note = DNA
cgaaagatggtgaatgtt

SEQ ID NO: 51 - DNA, 18 bp, synthetic construct
  source 1..18: mol_type = other DNA
  modified_base 1..5: mod_base = OTHER; note = 2'-MOE
  modified_base 14..18: mod_base = OTHER; note = 2'-MOE
  modified_base 1..18: mod_base = OTHER; note = Phosphorothioate backbone
  modified_base 1..18: mod_base = m5c
  misc_feature 6..13: note = DNA
tggtagtgcatgtggagt

SEQ ID NO: 52 - DNA, 18 bp, synthetic construct
  source 1..18: mol_type = other DNA
  modified_base 1..5: mod_base = OTHER; note = 2'-MOE
  modified_base 14..18: mod_base = OTHER; note = 2'-MOE
  modified_base 1..18: mod_base = OTHER; note = Phosphorothioate backbone
  modified_base 1..18: mod_base = m5c
  misc_feature 6..13: note = DNA
tgtgagtgggagaagttc

SEQ ID NO: 53 - DNA, 18 bp, synthetic construct
  source 1..18: mol_type = other DNA
  modified_base 1..5: mod_base = OTHER; note = 2'-MOE
  modified_base 14..18: mod_base = OTHER; note = 2'-MOE
  modified_base 1..18: mod_base = OTHER; note = Phosphorothioate backbone
  modified_base 1..18: mod_base = m5c
  misc_feature 6..13: note = DNA
cagtgctgctttcatgag

SEQ ID NO: 54 - DNA, 18 bp, synthetic construct
  source 1..18: mol_type = other DNA
  modified_base 1..5: mod_base = OTHER; note = 2'-MOE
  modified_base 14..18: mod_base = OTHER; note = 2'-MOE
  modified_base 1..18: mod_base = OTHER; note = Phosphorothioate backbone
  modified_base 1..18: mod_base = m5c
  misc_feature 6..13: note = DNA
caatgtaggtagttgcgg

SEQ ID NO: 55 - DNA, 18 bp, synthetic construct
  source 1..18: mol_type = other DNA
  modified_base 1..5: mod_base = OTHER; note = 2'-MOE
  modified_base 14..18: mod_base = OTHER; note = 2'-MOE
  modified_base 1..18: mod_base = OTHER; note = Phosphorothioate backbone
  modified_base 1..18: mod_base = m5c
  misc_feature 6..13: note = DNA
taagccattggcattgtc

SEQ ID NO: 56 - DNA, 18 bp, synthetic construct
  source 1..18: mol_type = other DNA
  modified_base 1..5: mod_base = OTHER; note = 2'-MOE
  modified_base 14..18: mod_base = OTHER; note = 2'-MOE
  modified_base 1..18: mod_base = OTHER; note = Phosphorothioate backbone
  modified_base 1..18: mod_base = m5c
  misc_feature 6..13: note = DNA
agaccatcgtgagataag

SEQ ID NO: 57 - DNA, 20 bp, synthetic construct
  source 1..20: mol_type = other DNA
  modified_base 1..5: mod_base = OTHER; note = 2'-MOE
  modified_base 16..20: mod_base = OTHER; note = 2'-MOE
  modified_base 1..20: mod_base = OTHER; note = Phosphodiester and Phosphorothioate backbone
  modified_base 1..20: mod_base = m5c
  misc_feature 6..15: note = DNA
agcggtgcatggggttgatc

SEQ ID NO: 58 - DNA, 20 bp, synthetic construct
  source 1..20: mol_type = other DNA
  modified_base 1..5: mod_base = OTHER; note = 2'-MOE
  modified_base 16..20: mod_base = OTHER; note = 2'-MOE
  modified_base 1..20: mod_base = OTHER; note = Phosphodiester and Phosphorothioate backbone
  modified_base 1..20: mod_base = m5c
  misc_feature 6..15: note = DNA
acgaaagatggtgaatgttg

SEQ ID NO: 59 - DNA, 20 bp, synthetic construct
  source 1..20: mol_type = other DNA
  modified_base 1..5: mod_base = OTHER; note = 2'-MOE
  modified_base 16..20: mod_base = OTHER; note = 2'-MOE
  modified_base 1..20: mod_base = OTHER; note = Phosphodiester and Phosphorothioate backbone
  modified_base 1..20: mod_base = m5c
  misc_feature 6..15: note = DNA
atggtagtgcatgtggagtc

SEQ ID NO: 60 - DNA, 20 bp, synthetic construct
  source 1..20: mol_type = other DNA
  modified_base 1..5: mod_base = OTHER; note = 2'-MOE
  modified_base 16..20: mod_base = OTHER; note = 2'-MOE
  modified_base 1..20: mod_base = OTHER; note = Phosphodiester and Phosphorothioate backbone
  modified_base 1..20: mod_base = m5c
  misc_feature 6..15: note = DNA
atgtgagtgggagaagttct

SEQ ID NO: 61 - DNA, 20 bp, synthetic construct
  source 1..20: mol_type = other DNA
  modified_base 1..5: mod_base = OTHER; note = 2'-MOE
  modified_base 16..20: mod_base = OTHER; note = 2'-MOE
  modified_base 1..20: mod_base = OTHER; note = Phosphodiester and Phosphorothioate backbone
  modified_base 1..20: mod_base = m5c
  misc_feature 6..15: note = DNA
ccagtgctgctttcatgagg

SEQ ID NO: 62 - DNA, 20 bp, synthetic construct
  source 1..20: mol_type = other DNA
  modified_base 1..5: mod_base = OTHER; note = 2'-MOE
  modified_base 16..20: mod_base = OTHER; note = 2'-MOE
  modified_base 1..20: mod_base = OTHER; note = Phosphodiester and Phosphorothioate backbone
  modified_base 1..20: mod_base = m5c
  misc_feature 6..15: note = DNA
acaatgtaggtagttgcgga

SEQ ID NO: 63 - DNA, 20 bp, synthetic construct
  source 1..20: mol_type = other DNA
  modified_base 1..5: mod_base = OTHER; note = 2'-MOE
  modified_base 16..20: mod_base = OTHER; note = 2'-MOE
  modified_base 1..20: mod_base = OTHER; note = Phosphodiester and Phosphorothioate backbone
  modified_base 1..20: mod_base = m5c
  misc_feature 6..15: note = DNA
ctaagccattggcattgtcg

SEQ ID NO: 64 - DNA, 20 bp, synthetic construct
  source 1..20: mol_type = other DNA
  modified_base 1..5: mod_base = OTHER; note = 2'-MOE
  modified_base 16..20: mod_base = OTHER; note = 2'-MOE
  modified_base 1..20: mod_base = OTHER; note = Phosphodiester and Phosphorothioate backbone
  modified_base 1..20: mod_base = m5c
  misc_feature 6..15: note = DNA
cagaccatcgtgagataagg

SEQ ID NO: 65 - DNA, 18 bp, synthetic construct
  source 1..18: mol_type = other DNA
  modified_base 1..5: mod_base = OTHER; note = 2'-MOE
  modified_base 14..18: mod_base = OTHER; note = 2'-MOE
  modified_base 1..18: mod_base = OTHER; note = Phosphodiester and Phosphorothioate backbone
  modified_base 1..18: mod_base = m5c
  misc_feature 6..13: note = DNA
gcggtgcatggggttgat

SEQ ID NO: 66 - DNA, 18 bp, synthetic construct
  source 1..18: mol_type = other DNA
  modified_base 1..5: mod_base = OTHER; note = 2'-MOE
  modified_base 14..18: mod_base = OTHER; note = 2'-MOE
  modified_base 1..18: mod_base = m5c
  modified_base 1..18: mod_base = OTHER; note = Phosphodiester and Phosphorothioate backbone
  misc_feature 6..13: note = DNA
cgaaagatggtgaatgtt

SEQ ID NO: 67 - DNA, 18 bp, synthetic construct
  source 1..18: mol_type = other DNA
  modified_base 1..5: mod_base = OTHER; note = 2'-MOE
  modified_base 14..18: mod_base = OTHER; note = 2'-MOE
  modified_base 1..18: mod_base = OTHER; note = Phosphodiester and Phosphorothioate backbone
  modified_base 1..18: mod_base = m5c
  misc_feature 6..13: note = DNA
tggtagtgcatgtggagt

SEQ ID NO: 68 - DNA, 18 bp, synthetic construct
  source 1..18: mol_type = other DNA
  modified_base 1..5: mod_base = OTHER; note = 2'-MOE
  modified_base 14..18: mod_base = OTHER; note = 2'-MOE
  modified_base 1..18: mod_base = OTHER; note = Phosphodiester and Phosphorothioate backbone
  modified_base 1..18: mod_base = m5c
  misc_feature 6..13: note = DNA
tgtgagtgggagaagttc

SEQ ID NO: 69 - DNA, 18 bp, synthetic construct
  source 1..18: mol_type = other DNA
  modified_base 1..5: mod_base = OTHER; note = 2'-MOE
  modified_base 14..18: mod_base = OTHER; note = 2'-MOE
  modified_base 1..18: mod_base = OTHER; note = Phosphodiester and Phosphorothioate backbone
  modified_base 1..18: mod_base = m5c
  misc_feature 6..13: note = DNA
cagtgctgctttcatgag

SEQ ID NO: 70 - DNA, 18 bp, synthetic construct
  source 1..18: mol_type = other DNA
  modified_base 1..5: mod_base = OTHER; note = 2'-MOE
  modified_base 14..18: mod_base = OTHER; note = 2'-MOE
  modified_base 1..18: mod_base = OTHER; note = Phosphodiester and Phosphorothioate backbone
  modified_base 1..18: mod_base = m5c
  misc_feature 6..13: note = DNA
caatgtaggtagttgcgg

SEQ ID NO: 71 - DNA, 18 bp, synthetic construct
  source 1..18: mol_type = other DNA
  modified_base 1..5: mod_base = OTHER; note = 2'-MOE
  modified_base 14..18: mod_base = OTHER; note = 2'-MOE
  modified_base 1..18: mod_base = OTHER; note = Phosphodiester and Phosphorothioate backbone
  modified_base 1..18: mod_base = m5c
  misc_feature 6..13: note = DNA
taagccattggcattgtc

SEQ ID NO: 72 - DNA, 18 bp, synthetic construct
  source 1..18: mol_type = other DNA
  modified_base 1..5: mod_base = OTHER; note = 2'-MOE
  modified_base 14..18: mod_base = OTHER; note = 2'-MOE
  modified_base 1..18: mod_base = OTHER; note = Phosphodiester and Phosphorothioate backbone
  modified_base 1..18: mod_base = m5c
  misc_feature 6..13: note = DNA
agaccatcgtgagataag

SEQ ID NO: 73 - DNA, 20 bp, synthetic construct
  source 1..20: mol_type = other DNA
  modified_base 5: mod_base = OTHER; note = uracil
  modified_base 17: mod_base = OTHER; note = uracil
  modified_base 19: mod_base = OTHER; note = uracil
gcggtgcatgggttgatctc

SEQ ID NO: 74 - DNA, 20 bp, synthetic construct
  source 1..20: mol_type = other DNA
  modified_base 1: mod_base = OTHER; note = uracil
  modified_base 4: mod_base = OTHER; note = uracil
  modified_base 18: mod_base = OTHER; note = uracil
tggtagtgcatgtagagtca

SEQ ID NO: 75 - DNA, 20 bp, synthetic construct
  source 1..20: mol_type = other DNA
  modified_base 4: mod_base = OTHER; note = uracil
cagtgctgctttcatgagga

SEQ ID NO: 76 - DNA, 20 bp, synthetic construct
  source 1..20: mol_type = other DNA
  modified_base 1..2: mod_base = OTHER; note = uracil
  modified_base 17: mod_base = OTHER; note = uracil
ttagcctttggcattatcgg

SEQ ID NO: 77 - DNA, 18 bp, synthetic construct
  source 1..18: mol_type = other DNA
gcggtgcatggggttgat

SEQ ID NO: 78 - DNA, 18 bp, synthetic construct
  source 1..18: mol_type = other DNA
cgaaagatggtgaatgtt

SEQ ID NO: 79 - DNA, 18 bp, synthetic construct
  source 1..18: mol_type = other DNA
tggtagtgcatgtggagt

SEQ ID NO: 80 - DNA, 18 bp, synthetic construct
  source 1..18: mol_type = other DNA
tgtgagtgggagaagttc

SEQ ID NO: 81 - DNA, 18 bp, synthetic construct
  source 1..18: mol_type = other DNA
cagtgctgctttcatgag

SEQ ID NO: 82 - DNA, 18 bp, synthetic construct
  source 1..18: mol_type = other DNA
caatgtaggtagttgcgg

SEQ ID NO: 83 - DNA, 18 bp, synthetic construct
  source 1..18: mol_type = other DNA
taagccattggcattgtc

SEQ ID NO: 84 - DNA, 18 bp, synthetic construct
  source 1..18: mol_type = other DNA
agaccatcgtgagataag